SEQUENCE LISTING (75 sequences)

SEQ ID NO: 1 - DNA, 20 bp, Artificial Sequence
  For PCR amplification of array-synthesized oligos
ttgtggaaacgtctcaaccg

SEQ ID NO: 2 - DNA, 20 bp, Artificial Sequence
  For PCR amplification of array-synthesized oligos
ctctagctccgtctcatgtt

SEQ ID NO: 3 - DNA, 65 bp, Artificial Sequence
  For construction of the sgRNAiBAR- expressing backbone
tatattcgaacgtctctaacagcatagcaagtttaaataaggcagtccgttatcaacttgaaaaa

SEQ ID NO: 4 - DNA, 66 bp, Artificial Sequence
  For construction of the sgRNAiBAR- expressing backbone
tatactcgagaaaaaaaagcaccgactcggtgccactttttcaagttgataacggactagccttat

SEQ ID NO: 5 - DNA, 20 bp, Artificial Sequence
  For PCR amplification of the sgRNAsiBAR-ANTXR1 coding region for NGS
aagcggaggacaggattggg

SEQ ID NO: 6 - DNA, 20 bp, Artificial Sequence
  For PCR amplification of the sgRNAsiBAR-ANTXR1 coding region for NGS
cctctgtggccctggagatg

SEQ ID NO: 7 - DNA, 20 bp, Artificial Sequence
  For PCR amplification of the T7E1 assay in CSPG4 gene
cacgggccctttaagaaggt

SEQ ID NO: 8 - DNA, 20 bp, Artificial Sequence
  For PCR amplification of the T7E1 assay in CSPG4 gene
ggacccacttctcactgtcg

SEQ ID NO: 9 - DNA, 23 bp, Artificial Sequence
  For PCR amplification of the T7E1 assay in MLH1 gene
gtgctcatcgttgccacatatta

SEQ ID NO: 10 - DNA, 21 bp, Artificial Sequence
  For PCR amplification of the T7E1 assay in MLH1 gene
tacgtgtaacagacaccttgc

SEQ ID NO: 11 - DNA, 18 bp, Artificial Sequence
  For PCR amplification of the T7E1 assay in MSH2 gene
ttgggtgtggtcgccgtg

SEQ ID NO: 12 - DNA, 19 bp, Artificial Sequence
  For PCR amplification of the T7E1 assay in MSH2 gene
cacaagcaccaacgttccg

SEQ ID NO: 13 - DNA, 25 bp, Artificial Sequence
  For PCR amplification of the T7E1 assay in MSH6 gene
tttttaaatactctttccttgcctg

SEQ ID NO: 14 - DNA, 20 bp, Artificial Sequence
  For PCR amplification of the T7E1 assay in MSH6 gene
agggcgtttccttcctagag

SEQ ID NO: 15 - DNA, 21 bp, Artificial Sequence
  For PCR amplification of the T7E1 assay in PMS2 gene(sgRNA1,2)
acactgtcttgggaaatgcaa

SEQ ID NO: 16 - DNA, 17 bp, Artificial Sequence
  For PCR amplification of the T7E1 assay in PMS2 gene(sgRNA1,2)
tggcagcgagacaaaac

SEQ ID NO: 17 - DNA, 20 bp, Artificial Sequence
  For PCR amplification of the T7E1 assay in PMS2 gene(sgRNA3)
ctcactgaacacaccatgcc

SEQ ID NO: 18 - DNA, 20 bp, Artificial Sequence
  For PCR amplification of the T7E1 assay in PMS2 gene(sgRNA3)
ggtctcactgtgttgcccag

SEQ ID NO: 19 - DNA, 55 bp, Artificial Sequence
  For PCR amplification of the sgRNAiBAR coding region for NGS
tacacgacgctcttccgatcttaagtagagtatcttgtggaaaggacgaaacacc

SEQ ID NO: 20 - DNA, 53 bp, Artificial Sequence
  For PCR amplification of the sgRNAiBAR coding region for NGS
agacgtgtgctcttccgatcttaagtagagagcttatcgataccgtcgacctc

SEQ ID NO: 21 - DNA, 56 bp, Artificial Sequence
  For PCR amplification of the sgRNAiBAR coding region for NGS
tacacgacgctcttccgatctatcatgcttatatcttgtggaaaggacgaaacacc

SEQ ID NO: 22 - DNA, 54 bp, Artificial Sequence
  For PCR amplification of the sgRNAiBAR coding region for NGS
agacgtgtgctcttccgatctatcatgcttaagcttatcgataccgtcgacctc

SEQ ID NO: 23 - DNA, 57 bp, Artificial Sequence
  For PCR amplification of the sgRNAiBAR coding region for NGS
tacacgacgctcttccgatctgatgcacatcttatcttgtggaaaggacgaaacacc

SEQ ID NO: 24 - DNA, 55 bp, Artificial Sequence
  For PCR amplification of the sgRNAiBAR coding region for NGS
agacgtgtgctcttccgatctgatgcacatctagcttatcgataccgtcgacctc

SEQ ID NO: 25 - DNA, 58 bp, Artificial Sequence
  For PCR amplification of the sgRNAiBAR coding region for NGS
tacacgacgctcttccgatctcgattgctcgactatcttgtggaaaggacgaaacacc

SEQ ID NO: 26 - DNA, 56 bp, Artificial Sequence
  For PCR amplification of the sgRNAiBAR coding region for NGS
agacgtgtgctcttccgatctcgattgctcgacagcttatcgataccgtcgacctc

SEQ ID NO: 27 - DNA, 59 bp, Artificial Sequence
  For PCR amplification of the sgRNAiBAR coding region for NGS
tacacgacgctcttccgatcttcgatagcaattctatcttgtggaaaggacgaaacacc

SEQ ID NO: 28 - DNA, 57 bp, Artificial Sequence
  For PCR amplification of the sgRNAiBAR coding region for NGS
agacgtgtgctcttccgatcttcgatagcaattcagcttatcgataccgtcgacctc

SEQ ID NO: 29 - DNA, 60 bp, Artificial Sequence
  For PCR amplification of the sgRNAiBAR coding region for NGS
tacacgacgctcttccgatctatcgatagttgctttatcttgtggaaaggacgaaacacc

SEQ ID NO: 30 - DNA, 58 bp, Artificial Sequence
  For PCR amplification of the sgRNAiBAR coding region for NGS
agacgtgtgctcttccgatctatcgatagttgcttagcttatcgataccgtcgacctc

SEQ ID NO: 31 - DNA, 61 bp, Artificial Sequence
  For PCR amplification of the sgRNAiBAR coding region for NGS
tacacgacgctcttccgatctgatcgatccagttagtatcttgtggaaaggacgaaacacc

SEQ ID NO: 32 - DNA, 59 bp, Artificial Sequence
  For PCR amplification of the sgRNAiBAR coding region for NGS
agacgtgtgctcttccgatctgatcgatccagttagagcttatcgataccgtcgacctc

SEQ ID NO: 33 - DNA, 20 bp, Artificial Sequence
  HPRT1_sgRNA 1
tcaccacgacgccagggctg

SEQ ID NO: 34 - DNA, 20 bp, Artificial Sequence
  HPRT1_sgRNA 2
gttatggcgacccgcagccc

SEQ ID NO: 35 - DNA, 20 bp, Artificial Sequence
  ITGB1_sgRNA 1
acacagcaaactgaactgat

SEQ ID NO: 36 - DNA, 20 bp, Artificial Sequence
  ITGB1_sgRNA 2
tacctgtttgagcaaacaca

SEQ ID NO: 37 - DNA, 20 bp, Artificial Sequence
  SRGAP2_sgRNA 1
cagccaaattcaaaaaggat

SEQ ID NO: 38 - DNA, 20 bp, Artificial Sequence
  SRGAP2_sgRNA 2
ccaaattcaaaaaggataag

SEQ ID NO: 39 - DNA, 20 bp, Artificial Sequence
  AKTIP_sgRNA 1
gcttgtagacatgctccaga

SEQ ID NO: 40 - DNA, 20 bp, Artificial Sequence
  AKTIP_sgRNA 2
cacgttatgaaccctttctg

SEQ ID NO: 41 - DNA, 20 bp, Artificial Sequence
  ACTR3C_sgRNA 1
caggactctacattgcagtt

SEQ ID NO: 42 - DNA, 20 bp, Artificial Sequence
  ACTR3C_sgRNA 2
cgttccaggactctacattg

SEQ ID NO: 43 - DNA, 20 bp, Artificial Sequence
  PPP1R17_sgRNA 1
tgatgtccactgagcaaatg

SEQ ID NO: 44 - DNA, 20 bp, Artificial Sequence
  PPP1R17_sgRNA 2
cagtggctgcatttgctcag

SEQ ID NO: 45 - DNA, 20 bp, Artificial Sequence
  ASCBG1_sgRNA 1
tgggcagccgtatccagctc

SEQ ID NO: 46 - DNA, 20 bp, Artificial Sequence
  ASCBG1_sgRNA 2
gcagatgccacgcaattctg

SEQ ID NO: 47 - DNA, 20 bp, Artificial Sequence
  CALM2_sgRNA 1
gtaggctgaccaactgactg

SEQ ID NO: 48 - DNA, 20 bp, Artificial Sequence
  CALM2_sgRNA 2
caatctgctcttcagtcagt

SEQ ID NO: 49 - DNA, 20 bp, Artificial Sequence
  TCF21_sgRNA 1
actcccccaaacatgtccac

SEQ ID NO: 50 - DNA, 20 bp, Artificial Sequence
  TCF21_sgRNA 2
cacatcgctgagggagccgg

SEQ ID NO: 51 - DNA, 20 bp, Artificial Sequence
  KIFAP3_sgRNA 1
caacacagatataacttccc

SEQ ID NO: 52 - DNA, 20 bp, Artificial Sequence
  KIFAP3_sgRNA 2
cagggaagttatatctgtgt

SEQ ID NO: 53 - DNA, 20 bp, Artificial Sequence
  FGF13_sgRNA 1
ttgttctctttgcagagcct

SEQ ID NO: 54 - DNA, 20 bp, Artificial Sequence
  FGF13_sgRNA 2
tctttgcagagcctcagctt

SEQ ID NO: 55 - DNA, 20 bp, Artificial Sequence
  DUPD1_sgRNA 1
cagatgagtaggcattcttg

SEQ ID NO: 56 - DNA, 20 bp, Artificial Sequence
  DUPD1_sgRNA 2
atgcctactcatctgccaag

SEQ ID NO: 57 - DNA, 20 bp, Artificial Sequence
  TECTA_sgRNA 1
tgaaagagacccaaattcta

SEQ ID NO: 58 - DNA, 20 bp, Artificial Sequence
  TECTA_sgRNA 2
ttcgcacttgtacagcacca

SEQ ID NO: 59 - DNA, 20 bp, Artificial Sequence
  GALR1_sgRNA 1
ggcggtcgggaacctcagcg

SEQ ID NO: 60 - DNA, 20 bp, Artificial Sequence
  GALR1_sgRNA 2
gttcccgaccgccagctcca

SEQ ID NO: 61 - DNA, 20 bp, Artificial Sequence
  OR51D1_sgRNA 1
tatgatagggaccaagagct

SEQ ID NO: 62 - DNA, 20 bp, Artificial Sequence
  OR51D1_sgRNA 2
atgatagggaccaagagctg

SEQ ID NO: 63 - DNA, 20 bp, Artificial Sequence
  MLH1_sgRNA 1
attacaacgaaaacagctga

SEQ ID NO: 64 - DNA, 20 bp, Artificial Sequence
  MLH1_sgRNA 2
ctgatggaaagtgtgcatac

SEQ ID NO: 65 - DNA, 20 bp, Artificial Sequence
  MSH2_sgRNA 1
cgcgctgctggccgcccggg

SEQ ID NO: 66 - DNA, 20 bp, Artificial Sequence
  MSH2_sgRNA 2
ggtcttgaacacctcccggg

SEQ ID NO: 67 - DNA, 20 bp, Artificial Sequence
  MSH2_sgRNA 3
gtgaggaggtttcgacatgg

SEQ ID NO: 68 - DNA, 20 bp, Artificial Sequence
  MSH6_sgRNA 1
gaagtacagcctaagacaca

SEQ ID NO: 69 - DNA, 20 bp, Artificial Sequence
  MSH6_sgRNA 2
agcctaagacacaaggatct

SEQ ID NO: 70 - DNA, 20 bp, Artificial Sequence
  PMS2_sgRNA 1
cgactgatgtttgatcacaa

SEQ ID NO: 71 - DNA, 20 bp, Artificial Sequence
  PMS2_sgRNA 2
agtttcaacctgagttaggt

SEQ ID NO: 72 - DNA, 20 bp, Artificial Sequence
  CSPG4_sgRNA 1
gagttaagtgcgcggacacc

SEQ ID NO: 73 - DNA, 20 bp, Artificial Sequence
  CSPG4_sgRNA 2
ccactcagctcccagctccc

SEQ ID NO: 74 - DNA, 20 bp, Artificial Sequence
  neg_sgRNA 1
caatagcaaaccggggcagt

SEQ ID NO: 75 - DNA, 20 bp, Artificial Sequence
  neg_sgRNA 2
gtgactccattaccaggctg

(19) United States
(12) Patent Application Publication
(10) Pub. No.: US 2022/0064633 A1
(43) Pub. Date: Mar. 3, 2022

(54) COMPOSITIONS AND METHODS FOR HIGHLY EFFICIENT GENETIC SCREENING USING BARCODED GUIDE RNA CONSTRUCTS

(71) Applicants: Peking University, Beijing (CN); Edigene Biotechnology Inc., Beijing (CN)

(72) Inventors: Wensheng WEI, Beijing (CN); Shiyou ZHU, Beijing (CN); Zhongzheng CAO, Beijing (CN); Zhiheng LIU, Beijing (CN); Yuan HE, Beijing (CN); Pengfei YUAN, Beijing (CN)

(73) Assignees: Peking University, Beijing (CN); Edigene Biotechnology Inc., Beijeing (CN)

(21) Appl. No.: 17/415,628

(22) PCT Filed: Dec. 20, 2019
(86) PCT No.: PCT/CN2019/127080

§ 371 Date: Jun. 17, 2021

(30) Foreign Application Priority Data
Dec. 20, 2018 (CN) PCT/CN2018/122383

(51) Int. Cl. C12N 15/10; C12N 15/11; C12N 9/22; C12N 15/86

(52) U.S. Cl. CPC C12N 15/1079; C12N 15/111; C12N 9/22; C12N 15/86; C12N 2740/15043; C12N 2320/12; C12N 2310/3519; C12N 2330/31; C12N 2310/20

(57) ABSTRACT

Compositions, kits and methods are provided for genetic screening using one or more sets of guide RNA constructs having internal barcodes (“iBAR”). Each set has three or more guide RNA constructs targeting the same genomic locus, but embedded with different iBAR sequences.

### FIELD OF THE INVENTION

The present invention relates to compositions, kits and methods for genetic screening using guide RNA constructs having internal barcodes (“iBARs”).

### BACKGROUND OF THE INVENTION

The CRISPR/Cas9 system enables editing at targeted genomic sites with high efficiency and specificity.¹⁻² One of its extensive applications is to identify functions of coding genes, non-coding RNAs and regulatory elements through high-throughput pooled screening in combination with next generation sequencing (“NGS”) analysis. By introducing a pooled single-guide RNA (“sgRNA”) or paired-guide RNA (“pgRNA”) library into cells expressing Cas9 or catalytically inactive Cas9 (dCas9) fused with effector domains, investigators can perform multifarious genetic screens by generating diverse mutations, large genomic deletions, transcriptional activation or transcriptional repression.³⁻⁹

To generate a high-quality cell library of gRNAs for any given pooled CRISPR screen, one must use a low multiplicity of infection (“MOI”) during cell library construction to ensure that each cell on average harbors less than one sgRNA or pgRNA to minimize the false-positive rate (FDR) of the screen.^{6,10,11} To further reduce the FDR and increase data reproducibility, in-depth coverage of gRNAs and multiple biological replicates are often necessary to obtain hit genes with high statistical significance,¹⁰ resulting in increased workload. Additional difficulties may arise when one performs a large number of genome-wide screens, when cell materials for library construction are limited, or when one conducts more challenging screens (i.e., in vivo screens) for which it is difficult to obtain experimental replicates or control the MOL There remains an urgent need for reliable and highly efficient screening strategy for large-scale target identification in eukaryotic cells.

The disclosures of all publications, patents, patent applications and published patent applications referred to herein are hereby incorporated herein by reference in their entirety.

### SUMMARY OF THE INVENTION

The present application provides guide RNA constructs, libraries, compositions and kits useful for genetic screening via a CRISPR-Cas gene-editing system, as well as genetic screening methods.

One aspect of the present application provides a set of sgRNA^{iBAR} constructs comprising three or more (e.g., four) sgRNA^{iBAR} constructs each comprising or encoding an sgRNA^{iBAR} wherein each sgRNA^{iBAR} has an sgRNA^{iBAR} sequence comprising a guide sequence and an internal barcode (“iBAR”) sequence, wherein each guide sequence is complementary to a target genomic locus, wherein the guide sequences for the three or more sgRNA^{iBAR} constructs are the same, wherein the iBAR sequence for each of the three or more sgRNA^{iBAR} constructs is different from each other, and wherein each sgRNA^{iBAR} is operable with a Cas protein to modify the target genomic locus. In some embodiments, each iBAR sequence comprises about 1-50 nucleotides, such as about 2-20 nucleotides or about 3-10 nucleotides. In some embodiments, each guide sequence comprises about 17-23 nucleotides.

In some embodiments according to any one of the sets of sgRNA^{iBAR} constructs described above, wherein each sgRNA^{iBAR} sequence comprises a first stem sequence and a second stem sequence, wherein the first stem sequence hybridizes with the second stem sequence to form a double-stranded RNA region that interacts with the Cas protein, and wherein the iBAR sequence is disposed between the first stem sequence and the second stem sequence. In some embodiments according to any one of the sets of sgRNA^{iBAR} constructs described above, wherein each sgRNA^{iBAR} sequence comprises in the 5′-to-3′ direction a first stem sequence and a second stem sequence, wherein the first stem sequence hybridizes with the second stem sequence to form a double-stranded RNA region that interacts with the Cas protein, and wherein the iBAR sequence is disposed between the 3′ end of the first stem sequence and the 5′ end of the second stem sequence.

In some embodiments according to any one of the sets of sgRNA^{iBAR} constructs described above, the Cas protein is Cas9. In some embodiments, each sgRNA^{iBAR} sequence comprises a guide sequence fused to a second sequence, wherein the second sequence comprises a repeat-anti-repeat stem loop that interacts with the Cas9. In some embodiments, the iBAR sequence of each sgRNA^{iBAR} sequence is disposed in the loop region of the repeat-anti-repeat stem loop. In some embodiments, the iBAR sequence of each sgRNA^{iBAR} sequence is inserted in the loop region of the repeat-anti-repeat stem loop. In some embodiments, the second sequence of each sgRNA^{iBAR} sequence further comprises a stem loop 1, stem loop 2, and/or stem loop 3. In some embodiments, the iBAR sequence of each sgRNA^{iBAR} sequence is disposed in the loop region of stem loop 1, stem loop 2 or stem loop 3. In some embodiments, the iBAR sequence of each sgRNA^{iBAR} sequence is inserted in the loop region of stem loop 1, stem loop 2 or stem loop 3.

In some embodiments according to any one of the sets of sgRNA^{iBAR} constructs described above, each sgRNA^{iBAR} construct is a plasmid. In some embodiments, each sgRNA^{iBAR} construct is a viral vector, such as a lentiviral vector.

One aspect of the present application provides an sgRNA^{iBAR} library comprising a plurality of sets of sgRNA^{iBAR} constructs according to any one of the sets of sgRNA^{iBAR} constructs described above, wherein each set corresponds to a guide sequence complementary to a different target genomic locus. In some embodiments, the sgRNA^{iBAR} library comprises at least about 1000 (e.g., at least about 2000, 5000, 10000, 15000, 20000, or more) sets of sgRNA^{iBAR} constructs. In some embodiments, the iBAR sequences for at least two sets of sgRNA^{iBAR} constructs are the same. In some embodiments, different sets of sgRNA^{iBAR} constructs have different combinations of iBAR sequences.

One aspect of the present application provides a method of preparing an sgRNA^{iBAR} library comprising a plurality of sets of sgRNA^{iBAR} constructs, wherein each set corresponds to one of a plurality of guide sequences each complementary to a different target genomic locus, wherein the method comprises: a) designing three or more (e.g., four) sgRNA^{iBAR} constructs for each guide sequence, wherein each sgRNA^{iBAR} construct comprises or encodes an sgRNA^{iBAR} having an sgRNA^{iBAR} sequence comprising the corresponding guide sequence and an iBAR sequence, wherein the iBAR sequence corresponding to each of the three or more sgRNA^{iBAR} constructs is different from each other, and wherein each sgRNA^{iBAR} is operable with a Cas protein to modify the corresponding target genomic locus; and b) synthesizing each sgRNA^{iBAR} construct, thereby producing the sgRNA^{iBAR} library. In some embodiments, the method further comprises providing the plurality of guide sequences.

In some embodiments according to any one of the methods of preparation described above, each iBAR sequence comprises about 1-50 nucleotides, such as about 2-20 nucleotides or about 3-10 nucleotides. In some embodiments, each guide sequence comprises about 17-23 nucleotides.

In some embodiments according to any one of the methods of preparation described above, wherein each sgRNA^{iBAR} sequence comprises a first stem sequence and a second stem sequence, wherein the first stem sequence hybridizes with the second stem sequence to form a double-stranded RNA region that interacts with the Cas protein, and wherein the iBAR sequence is disposed between the first stem sequence and the second stem sequence. In some embodiments according to any one of the methods of preparation described above, wherein each sgRNA^{iBAR} sequence comprises in the 5′-to-3′ direction a first stem sequence and a second stem sequence, wherein the first stem sequence hybridizes with the second stem sequence to form a double-stranded RNA region that interacts with the Cas protein, and wherein the iBAR sequence is disposed between the 3′ end of the first stem sequence and the 5′ end of the second stem sequence.

In some embodiments according to any one of the methods of preparation described above, the Cas protein is Cas9. In some embodiments, each sgRNA^{iBAR} sequence comprises a guide sequence fused to a second sequence, wherein the second sequence comprises a repeat-anti-repeat stem loop that interacts with the Cas9. In some embodiments, the iBAR sequence of each sgRNA^{iBAR} sequence is disposed in the loop region of the repeat-anti-repeat stem loop. In some embodiments, the iBAR sequence of each sgRNA^{iBAR} sequence is inserted in the loop region of the repeat-anti-repeat stem loop. In some embodiments, the second sequence of each sgRNA^{iBAR} sequence further comprises a stem loop 1, stem loop 2, and/or stem loop 3. In some embodiments, the iBAR sequence of each sgRNA^{iBAR} sequence is disposed in the loop region of stem loop 1, stem loop 2 or stem loop 3. In some embodiments, the iBAR sequence of each sgRNA^{iBAR} sequence is inserted in the loop region of stem loop 1, stem loop 2 or stem loop 3.

In some embodiments according to any one of the methods of preparation described above, each sgRNA^{iBAR} construct is a plasmid. In some embodiments, each sgRNA^{iBAR} construct is a viral vector, such as a lentiviral vector.

Also provided are sgRNA^{iBAR} libraries prepared using the method according to any one of the methods of preparation described above, as well as compositions comprising any one of the sets of sgRNA^{iBAR} constructs described above, or any one of the sgRNA^{iBAR} libraries described above.

Another aspect of the present application provides a method of screening for a genomic locus that modulates a phenotype of a cell, comprising: a) contacting an initial population of cells with i) the sgRNA^{iBAR} library according to any one of the sgRNA^{iBAR} libraries described above; and optionally ii) a Cas component comprising a Cas protein or a nucleic acid encoding the Cas protein under a condition that allows introduction of the sgRNA^{iBAR} constructs and the optional Cas component into the cells to provide a modified population of cells; b) selecting a population of cells having a modulated phenotype from the modified population of cells to provide a selected population of cells; c) obtaining sgRNA^{iBAR} sequences from the selected population of cells; d) ranking the corresponding guide sequences of the sgRNA^{iBAR} sequences based on sequence counts, wherein the ranking comprises adjusting the rank of each guide sequence based on data consistency among the iBAR sequences in the sgRNA^{iBAR} sequences corresponding to the guide sequence; and e) identifying the genomic locus corresponding to a guide sequence ranked above a predetermined threshold level. In some embodiments, the cell is a eukaryotic cell, such as a mammalian cell. In some embodiments, the initial population of cells expresses a Cas protein.

In some embodiments according to any one of the methods of screening described above, each sgRNA^{iBAR} construct is a viral vector, and wherein the sgRNA^{iBAR} library is contacted with the initial population of cells at a multiplicity of infection (MOI) of more than about 2 (e.g., 3, 4, 5, 6, 7, 8, 9, 10, or higher). In some embodiments, more than about 95% (e.g., more than about 97%, 98%, 99% or higher) of the sgRNA^{iBAR} constructs in the sgRNA^{iBAR} library are introduced into the initial population of cells. In some embodiments, the screening is carried out at more than about 1000-fold (e.g., 2000-fold, 3000-fold, 5000-fold or higher) coverage.

In some embodiments according to any one of the methods of screening described above, the screening is positive screening. In some embodiments, the screening is negative screening.

In some embodiments according to any one of the methods of screening described above, the phenotype is protein expression, RNA expression, protein activity, or RNA activity. In some embodiments, the phenotype is selected from the group consisting of cell death, cell growth, cell motility, cell metabolism, drug resistance, drug sensitivity, and response to a stimulus. In some embodiments, the phenotype is response to a stimulus, and wherein the stimulus is selected from the group consisting of a hormone, a growth factor, an inflammatory cytokine, an anti-inflammatory cytokine, a drug, a toxin, and a transcription factor.

In some embodiments according to any one of the methods of screening described above, the sgRNA^{iBAR} sequences are obtained by genome sequencing or RNA sequencing. In some embodiments, the sgRNA^{iBAR} sequences are obtained by next-generation sequencing.

In some embodiments according to any one of the methods of screening described above, the sequence counts are subject to median ratio normalization followed by mean-variance modeling. In some embodiments, the variance of each guide sequence is adjusted based on data consistency among the iBAR sequences in the sgRNA^{iBAR} sequences corresponding to the guide sequence. In some embodiments, the sequence counts obtained from the selected population of cells are compared to corresponding sequence counts obtained from a population of control cells to provide fold changes. In some embodiments, the data consistency among the iBAR sequences in the sgRNA^{iBAR} sequences corresponding to each guide sequence is determined based on the direction of the fold change of each iBAR sequence, wherein the variance of the guide sequence is increased if the fold changes of the iBAR sequences are in opposite directions with respect to each other.

In some embodiments according to any one of the methods of screening described above, the method further comprises validating the identified genomic locus.

Also provided are kits and articles of manufacture for screening a genomic locus that modulates a phenotype of a cell, comprising any one of the sgRNA^{iBAR} libraries described above. In some embodiments, the kit or article of manufacture further comprises a Cas protein or a nucleic acid encoding the Cas protein.

### BRIEF DESCRIPTION OF THE DRAWINGS

FIGS. 1A-1E show an exemplary CRISPR/Cas-based screening using sgRNA^{iBAR} constructs. FIG. 1A shows a schematic diagram of an sgRNA^{iBAR} with an internal barcode (iBAR). A 6-nt barcode (iBAR₆) was embedded in the tetra loop of the sgRNA scaffold. FIG. 1B shows results from a CRISPR/Cas-based screening experiment using a library of sgRNA constructs targeting a single gene (ANTXR1; referred herein as “sgRNA^{iBAR-ANTXR1}”) but having all 4,096 iBAR₆ sequences. Control sgRNA constructs (“sgRNA^{non-targeting”)} have a guide sequence not targeting ANTXR1, but have the corresponding iBAR₆ sequences. Fold changes between the reference and toxin (PA/LFnDTA)-treatment groups were calculated using the normalized abundance of each sgRNA^{iBAR-ANTXR1}. A density plot showing the fold changes of the sgRNA^{iBAR-ANTXR1}, non-barcoded sgRNA^{ANTXR1} and non-targeting sgRNAs is presented. Pearson correlation is calculated (“Con”). FIG. 1C shows effects of nucleotide identities at each position of the iBAR₆ on editing efficiency of sgRNAs. FIG. 1D shows indels generated by sgRNA^{iBAR-ANTXR1} having six barcodes associated with least cell resistance against PA/LFnDTA in the screening experiment. Percentages of cleavage efficiency in the T7E1 assay were measured using Image Lab software, and data are presented as the mean±s.d. (n=3). All primers used are listed in Table 1. FIG. 1E shows results of an MTT viability assay, which demonstrate decreased susceptibility of cells edited by the indicated sgRNA^{iBAR-ANTXR1} against PA/LFnDTA.

FIG. 2 shows CRISPR screening of a collection of sgRNAs^{iBAR-ANTXR1} containing all 4,096 types of iBAR₆ sequences categorized into three groups according to the GC contents of the iBAR sequences. GC contents in the three groups are: high (100-66%), medium (66-33%) and low (33-0%). The rankings of two biological replicates are displayed.

FIGS. 3A-3D show evaluation of the effects of iBAR sequences on sgRNA activity. Indels generated by sgRNA1^{iBAR-CSPG4} (FIG. 3A), sgRNA2^{iBAR-CSPG4} (FIG. 3B), sgRNA2^{iBAR-MLH1} (FIG. 3C) and sgRNA3^{iBAR-MSH2} (FIG. 3D) associated with six barcodes that appeared to be the worst in conferring cell resistance to PA/LFnDTA from the above screening as well as with GTTTTTT that was supposed to be termination signal for U6 promoter. Percentages of cleavage efficiency in the T7E1 assay were measured using Image Lab software, and data are presented as the mean±s.d. (n=3). All primers used are listed in Table 1.

FIG. 4 shows a schematic of CRISPR-pooled screening using an sgRNA^{iBAR} library. For a given sgRNA^{iBAR} library, four different iBAR₆s were randomly assigned to each sgRNA. The sgRNA^{iBAR} library was introduced into target cells through lentiviral infection with a high MOI (i.e., ˜3). After library screening, sgRNAs with their associated iBARs from enriched cells were determined through NGS. For data analysis, median ratio normalization was applied, followed by mean-variance modelling. The variance of sgRNA^{iBAR} was determined based on the fold-change consistency of all iBARs assigned to the same sgRNA. The P value of each sgRNA^{iBAR} was calculated using the mean and modified variance. Robust rank aggregation (RRA) scores of all genes were considered to identify hit genes. A lower RRA score corresponded to a stronger enrichment of the hit genes.

FIG. 5 shows DNA sequences of the designed oligos. An array-synthesized 85-nt DNA oligo contains coding sequences of sgRNAs and barcodeiBAR₆. The left and right arms are used for primer targeting for amplification. BsmBI sites are used for cloning pooled, barcoded sgRNAs into the final expressing backbone.

FIGS. 6A-6F show screening results for essential genes involved in TcdB toxicity at MOI of 0.3, 3 and 10 in HeLa cells. FIGS. 6A and 6B show Screening scores of identified genes (FDR<0.15) calculated by MAGeCK (FIG. 6A) and by MAGeCK^{iBAR} (FIG. 6B) at MOI of 0.3. FIGS. 6C and 6D show screening scores of identified genes (FDR<0.15) calculated by MAGeCK (FIG. 6C) and by MAGeCK^{iBAR} (FIG. 6D) at MOI of 3. FIGS. 6E-6F show screening scores of identified genes (FDR<0.15) calculated by MAGeCK (FIG. 6E) and by MAGeCK^{iBAR} (FIG. 6F) at MOI of 10. Negative control genes are labelled with dark dots on the bottom of Y-axis. Rankings of identified candidates in each biological replicate through MAGeCK and MAGeCK^{iBAR} were presented.

FIGS. 7A-7H show sgRNA^{iBAR} read counts for CSPG4 targeting constructs (FIG. 7A), SPPL3 targeting constructs (FIG. 7B), UGP2 targeting constructs (FIG. 7C), KATNAL2 targeting constructs (FIG. 7D), HPRT1 targeting constructs (FIG. 7E), RNF212B targeting constructs (FIG. 7F), SBNO2 targeting constructs (FIG. 7G) and ERAS targeting constructs (FIG. 7H) before (Ctrl) and after (Exp) TcdB screening at MOI of 10 calculated by MAGeCK in two replicates.

FIGS. 8A-8C show sgRNA distribution and coverage in different samples. FIG. 8A shows sgRNA^{iBAR} distribution of the reference and 6-TG treatment groups. The horizontal axis indicates the normalized RPM in log 10, and the vertical axis indicates the number of sgRNAs. FIG. 8B shows sgRNA coverage of reference samples. The vertical axis indicates the sgRNA proportion vs. design. FIG. 8C shows proportions of sgRNAs carrying different numbers of designed iBARs in the library.

FIG. 9 shows Pearson correlation of log 10(fold change) of all genes between two biological replicates after 6-TG screening at an MOI of 3.

FIG. 10 shows a mean-variance model of all the sgRNAs^{iBAR} after variance adjustment using MAGeCK^{iBAR} analysis.

FIGS. 11A-11G shows comparison of the CRISPR^{iBAR} and conventional CRISPR pooled screens for the identification of human genes important for 6-TG-mediated cytotoxicity in HeLa cells. FIGS. 11A-11B shows screening scores of the top-ranked genes calculated by MAGeCK^{iBAR} (FIG. 11A) and by MAGeCK (FIG. 11B). Identified candidates (FDR<0.15) were labelled, and only top 10 hits were labelled for MAGeCK^{iBAR} screens. Negative control genes were labelled with dark dots on the bottom of Y-axis. FIG. 11C shows validation of reported genes (MLH1, MSH2, MSH6 and PMS2) involved in 6-TG cytotoxicity. FIG. 11D shows Spearman correlation coefficient of the top 20 positively selected genes between two biological replicates using MAGeCK^{iBAR} (left) or conventional MAGeCK analysis (right). FIG. 11E shows validation of top candidate genes isolated by either MAGeCK^{iBAR} or MAGeCK analysis. Mini-pooled sgRNAs targeting each gene were delivered to cells through lentiviral infection. Transduced cells were cultured for an additional ten days before 6-TG treatment. Data are presented as the mean±S.E.M. (n=5). P values were calculated using Student's t-test. *P<0.05; **P<0.01; ***P<0.001; NS, not significant. The sgRNA sequences for validation are listed in Table 3. FIGS. 11F-11G show sgRNA^{iBAR} read counts for HPRT1 targeting constructs (FIG. 11F) and FGF13 targeting constructs (FIG. 11G) before (Ctrl) and after (Exp) 6-TG screening in two replicates.

FIG. 12 shows efficiency of original designed sgRNAs targeting MLH1, MSH2, MSH6 and PMS2. Percentages of cleavage efficiency in the T7E1 assay were measured using Image Lab software, and data are presented as the mean±s.d. (n=3). All primers used are listed in Table 1.

FIG. 13 shows fold changes of each sgRNA^{iBAR} targeting the indicated top candidate genes (HPRT1, ITGB1, SRGAP2 and AKTIP) in two experimental replicates. Ctrl and Exp represent the samples before and after 6-TG treatment, respectively.

FIGS. 14A-14I shows sgRNA^{iBAR} read counts for targeting ITGB1 (FIG. 14A), SRGAP2 (FIG. 14B), AKTIP (FIG. 14C), ACTR3C (FIG. 14D), PPP1R17 (FIG. 14E), ACSBG1 (FIG. 14F), CALM2 (FIG. 14G), TCF21 (FIG. 14H) and KIFAP3 (FIG. 14I) in two replicates. Ctrl and Exp represent the samples before and after 6-TG treatment, respectively.

FIGS. 15A-15F shows sgRNA^{iBAR} read counts for targeting GALR1 (FIG. 15A), DUPD1 (FIG. 15B), TECTA (FIG. 15C), OR51D1 (FIG. 15D), Neg89 (FIG. 15E) and Neg67 (FIG. 15F) in two replicates. Ctrl and Exp represent the samples before and after 6-TG treatment, respectively.

FIG. 16 shows normalized sgRNA read counts of HPRT1, FGF13, GALR1 and Neg67 via conventional analysis in two experimental replicates. Ctrl and Exp represent the samples before and after 6-TG treatment, respectively.

FIG. 17 shows assessment of screen performance through MAGeCK and MAGeCK^{iBAR} analyses by using gold standard essential genes as determined by ROC curves. The AUC (area under curve) values were shown. Dashed lines indicate the performance of a random classification model.

FIG. 18 shows effects of different lengths of iBARs on sgRNA activity. Indels were generated by sgRNA1^{CSPG4} and sgRNA1^{iBAR-CSPG4} with different lengths of barcodes as indicated. Percentages of cleavage efficiency in the T7E1 assay were measured using Image Lab software, and data are presented as the mean±s.d. (n=3). All primers used are listed in Table 1.

### DETAILED DESCRIPTION OF THE INVENTION

The present application provides compositions and methods for genetic screening using guide RNA sets having internal barcodes (iBARs). Each set of guide RNAs targets a specific genomic locus, and is associated with three or more iBAR sequences. A guide RNA library comprising a plurality of guide RNA sets each targeting a different genomic locus may be used in a CRISPR/Cas-based screen to identify genomic loci that modulate a phenotype in a pooled cell library. Screening methods described herein have reduced false discovery rates because the iBAR sequences allow analysis of replicate gene-edited samples corresponding to each set of guide RNA constructs in a single experiment. The low false discovery rates also enable high-efficiency cell library generation by viral transduction of the guide RNA library to cells at a high multiplicity of infection (MOI).

Experimental data described herein demonstrate that the iBAR methods are especially advantageous in high-throughput screens. Conventional CRISPR/Cas screening methods are often labor intensive because they require low multiplicity of infection (MOI) for lentiviral transduction when generating cell libraries and multiple biological replicates to minimize the false discovery rate. In contrast, the iBAR methods produce screening results with much lower false-positive and false-negative rates, and allow cell library generation using a high MOL For example, compared to a conventional CRISPR/Cas screen with a low MOI of 0.3, the iBAR methods can reduce the starting cell numbers for more than 20-fold (e.g., at an MOI of 3) to more than 70-fold (e.g., at an MOI of 10), while maintaining high efficiency and accuracy. The iBAR system is particularly useful for cell-based screens in which the cells are available in limited quantities, or for in vivo screens in which viral infection to specific cells or tissues is difficult to control at low MOI.

Accordingly, one aspect of the present application provides a set of sgRNA^{iBAR} constructs comprising three or more (e.g., four) sgRNA^{iBAR} constructs each comprising or encoding an sgRNA^{iBAR} wherein each sgRNA^{iBAR} has an sgRNA^{iBAR} sequence comprising a guide sequence and an internal barcode (“iBAR”) sequence, wherein each guide sequence is complementary to a target genomic locus, wherein the guide sequences for the three or more sgRNA^{iBAR} constructs are the same, wherein the iBAR sequence for each of the three or more sgRNA^{iBAR} constructs is different from each other, and wherein each sgRNA^{iBAR} is operable with a Cas protein to modify the target genomic locus.

One aspect of the present application provides an sgRNA^{iBAR} library comprising a plurality of sets of sgRNA^{iBAR} constructs, wherein each set of sgRNA^{iBAR} constructs comprises three or more sgRNA^{iBAR} constructs each comprising or encoding an sgRNA^{iBAR}, wherein each sgRNA^{iBAR} has an sgRNA^{iBAR} sequence comprising a guide sequence and an iBAR sequence, wherein each guide sequence is complementary to a target genomic locus, wherein the guide sequences for the three or more sgRNA^{iBAR} constructs are the same, wherein the iBAR sequence for each of the three or more sgRNA^{iBAR} constructs is different from each other, wherein each sgRNA^{iBAR} is operable with a Cas protein to modify the target genomic locus, and wherein each set of sgRNA^{iBAR} constructs corresponds to a guide sequence complementary to a different target genomic locus.

Also provided is a method of screening for a genomic locus that modulates a phenotype of a cell, comprising: a) contacting an initial population of cells with i) an sgRNA^{iBAR} library comprising a plurality of sets of sgRNA^{iBAR} constructs, wherein each set of sgRNA^{iBAR} constructs comprises three or more sgRNA^{iBAR} constructs each comprising or encoding an sgRNA^{iBAR} wherein each sgRNA^{iBAR} has an sgRNA^{iBAR} sequence comprising a guide sequence and an iBAR sequence, wherein each guide sequence is complementary to a target genomic locus, wherein the guide sequences for the three or more sgRNA^{iBAR} constructs are the same, wherein the iBAR sequence for each of the three or more sgRNA^{iBAR} constructs is different from each other, wherein each sgRNA^{iBAR} is operable with a Cas protein to modify the target genomic locus, and wherein each set of sgRNA^{iBAR} constructs corresponds to a guide sequence complementary to a different target genomic locus; and optionally ii) a Cas component comprising a Cas protein or a nucleic acid encoding the Cas protein under a condition that allows introduction of the sgRNA^{iBAR} constructs and the optional Cas component into the cells to provide a modified population of cells; b) selecting a population of cells having a modulated phenotype from the modified population of cells to provide a selected population of cells; c) obtaining sgRNA^{iBAR} sequences from the selected population of cells; d) ranking the corresponding guide sequences of the sgRNA^{iBAR} sequences based on sequence counts, wherein the ranking comprises adjusting the rank of each guide sequence based on data consistency among the iBAR sequences in the sgRNA^{iBAR} sequences corresponding to the guide sequence; and e) identifying the genomic locus corresponding to a guide sequence ranked above a predetermined threshold level.

### Definition

The present invention will be described with respect to particular embodiments and with reference to certain drawings but the invention is not limited thereto. Any reference signs in the claims shall not be construed as limiting the scope. In the drawings, the size of some of the elements may be exaggerated and not drawn on scale for illustrative purposes. Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

As used herein, “internal barcode” or “iBAR” refers to an index inserted into or appended to a molecule, which is useful for tracing the identity and performance of the molecule. The iBAR can be, for example, a short nucleotide sequence inserted in or appended to a guide RNA for a CRISPR/Cas system, as exemplified by the present invention. Multiple iBARs can be used to trace the performance of a single guide RNA sequence within one experiment, thereby providing replicate data for statistical analysis without having to repeat the experiment.

The expression “iBAR sequence is disposed in a loop region” means the iBAR sequence is inserted between any two nucleotides of the loop region, inserted at the 5′ or 3′ end of the loop region, or replaces one or more nucleotides of the loop region.

“CRISPR system” or “CRISPR/Cas system” refers collectively to transcripts and other elements involved in the expression and/or directing the activity of CRISPR-associated (“Cas”) genes. For example, a CRISPR/Cas system may include sequences encoding a Cas gene, a tracr (trans-activating CRISPR) sequence (e.g., tracrRNA or an active partial tracrRNA), a tracr-mate sequence (e.g., encompassing a “direct repeat” and a tracrRNA-processed partial direct repeat in an endogenous CRISPR system), a guide sequence (also referred to as a “spacer” in an endogenous CRISPR system), and other sequences and transcripts derived from a CRISPR locus.

In the context of formation of a CRISPR complex, “target sequence” refers to a sequence to which a guide sequence is designed to have complementarity, where hybridization between a target sequence and a guide sequence promotes the formation of a CRISPR complex. Full complementarity is not necessarily required, provided there is sufficient complementarity to cause hybridization and promote formation of a CRISPR complex. A target sequence may comprise any polynucleotide, such as DNA or RNA polynucleotides. A CRISPR complex may comprise a guide sequence hybridized to a target sequence and complexed with one or more Cas proteins.

The term “guide sequence” refers to a contiguous sequence of nucleotides in a guide RNA which has partial or complete complementarity to a target sequence in a target polynucleotide and can hybridize to the target sequence by base pairing facilitated by a Cas protein. In a CRISPR/Cas9 system, a target sequence is adjacent to a PAM site. The PAM sequence, and its complementary sequence on the other strand, together constitutes a PAM site.

The terms “single guide RNA,” “synthetic guide RNA” and “sgRNA” are used interchangeably and refer to a polynucleotide sequence comprising a guide sequence and any other sequence necessary for the function of the sgRNA and/or interaction of the sgRNA with one or more Cas proteins to form a CRISPR complex. In some embodiments, an sgRNA comprises a guide sequence fused to a second sequence comprising a tracr sequence derived from a tracr RNA and a tracr mate sequence derived from a crRNA. A tracr sequence may contain all or part of the sequence from the tracrRNA of a naturally-occurring CRISPR/Cas system. The term “guide sequence” refers to the nucleotide sequence within the guide RNA that specifies the target site and may be used interchangeably with the term “guide” or “spacer.” The term “tracr mate sequence” may also be used interchangeably with the term “direct repeat(s).” “sgRNA^{iBAR}” as used herein refers to a single-guide RNA having an iBAR sequence.

The term “operable with a Cas protein” means that a guide RNA can interact with the Cas protein to form a CRISPR complex.

As used herein the term “wild type” is a term of the art understood by skilled persons and means the typical form of an organism, strain, gene or characteristic as it occurs in nature as distinguished from mutant or variant forms.

As used herein the term “variant” should be taken to mean the exhibition of qualities that have a pattern that deviates from what occurs in nature.

“Complementarity” refers to the ability of a nucleic acid to form hydrogen bond(s) with another nucleic acid sequence by either traditional Watson-Crick base pairing or other non-traditional types. A percent complementarity indicates the percentage of residues in a nucleic acid molecule which can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary). “Perfectly complementary” means that all the contiguous residues of a nucleic acid sequence will hydrogen bond with the same number of contiguous residues in a second nucleic acid sequence. “Substantially complementary” as used herein refers to a degree of complementarity that is at least 60%, 65%, 70%, 75%, 80%, 85%, 90%, 95%, 97%, 98%, 99%, or 100% over a region of 8, 9, 10, 11, 12, 13, 14, 15, 16, 17, 18, 19, 20, 21, 22, 23, 24, 25, 30, 35, 40, 45, 50, or more nucleotides, or refers to two nucleic acids that hybridize under stringent conditions.

As used herein, “stringent conditions” for hybridization refer to conditions under which a nucleic acid having complementarity to a target sequence predominantly hybridizes with the target sequence, and substantially does not hybridize to non-target sequences. Stringent conditions are generally sequence-dependent, and vary depending on a number of factors. In general, the longer the sequence, the higher the temperature at which the sequence specifically hybridizes to its target sequence. Non-limiting examples of stringent conditions are described in detail in Tijssen (1993), Laboratory Techniques In Biochemistry And Molecular Biology-Hybridization With Nucleic Acid Probes Part 1, Second Chapter “Overview of principles of hybridization and the strategy of nucleic acid probe assay”, Elsevier, N.Y.

“Hybridization” refers to a reaction in which one or more polynucleotides react to form a complex that is stabilized via hydrogen bonding between the bases of the nucleotide residues. The hydrogen bonding may occur by Watson Crick base pairing, Hoogstein binding, or in any other sequence specific manner. The complex may comprise two strands forming a duplex structure, three or more strands forming a multi stranded complex, a single self-hybridizing strand, or any combination of these. A hybridization reaction may constitute a step in a more extensive process, such as the initiation of PCR, or the cleavage of a polynucleotide by an enzyme. A sequence capable of hybridizing with a given sequence is referred to as the “complement” of the given sequence.

“Construct” as used herein refers to a nucleic acid molecule (e.g., DNA or RNA). For example, when used in the context of an sgRNA, a construct refers to a nucleic acid molecule comprising the sgRNA molecule or a nucleic acid molecule encoding the sgRNA. When used in the context of a protein, a construct refers to a nucleic acid molecule comprising a nucleotide sequence that can be transcribed to an RNA or expressed as a protein. A construct may contain necessary regulatory elements operably linked to the nucleotide sequence that allow transcription or expression of the nucleotide sequence when the construct is present in a host cell.

“Operably linked” as used herein means that expression of a gene is under the control of a regulatory element (e.g., a promoter) with which it is spatially connected. A regulatory element may be positioned 5′ (upstream) or 3′ (downstream) to a gene under its control. The distance between the regulatory element (e.g., promoter) and a gene may be approximately the same as the distance between that regulatory element (e.g., promoter) and a gene it naturally controls and from which the regulatory element is derived. As it is known in the art, variation in this distance may be accommodated without loss of function in the regulatory element (e.g., promoter).

The term “vector” is used to describe a nucleic acid molecule that may be engineered to contain a cloned polynucleotide or polynucleotides that may be propagated in a host cell. Vectors include, but are not limited to, nucleic acid molecules that are single-stranded, double-stranded, or partially double-stranded; nucleic acid molecules that comprise one or more free ends, no free ends (e.g. circular); nucleic acid molecules that comprise DNA, RNA, or both; and other varieties of polynucleotides known in the art. One type of vector is a “plasmid,” which refers to a circular double-stranded DNA loop into which additional DNA segments can be inserted, such as by standard molecular cloning techniques. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operably linked. Such vectors are referred to herein as “expression vectors.” Recombinant expression vectors can comprise a nucleic acid of the invention in a form suitable for expression of the nucleic acid in a host cell, which means that the recombinant expression vectors include one or more regulatory elements, which may be selected on basis of the host cells to be used for expression, that is operably linked to the nucleic acid sequence to be expressed.

A “host cell” refers to a cell that may be or has been a recipient of a vector or isolated polynucleotide. Host cells may be prokaryotic cells or eukaryotic cells. In some embodiments, the host cell is a eukaryotic cell that can be cultured in vitro and modified using the methods described herein. The term “cell” includes the primary subject cell and its progeny.

“Multiplicity of infection” or “MOI” are used interchangeably herein to refer to a ratio of agents (e.g., phage, virus, or bacteria) to their infection targets (e.g., cell or organism). For example, when referring to a group of cells inoculated with viral particles, the multiplicity of infection or MOI is the ratio between the number of viral particles (e.g., viral particles comprising an sgRNA library) and the number of target cells present in a mixture during viral transduction.

A “phenotype” of a cell as used herein refers to an observable characteristic or trait of a cell, such as its morphology, development, biochemical or physiological property, phenology, or behavior. A phenotype may result from expression of genes in a cell, influence from environmental factors, or interactions between the two.

Where the term “comprising” is used in the present description and claims, it does not exclude other elements or steps.

It is understood that embodiments of the invention described herein include “consisting” and/or “consisting essentially of” embodiments.

Reference to “about” a value or parameter herein includes (and describes) variations that are directed to that value or parameter per se. For example, description referring to “about X” includes description of “X”.

As used herein, reference to “not” a value or parameter generally means and describes “other than” a value or parameter. For example, the method is not used to treat cancer of type X means the method is used to treat cancer of types other than X.

The term “about X-Y” used herein has the same meaning as “about X to about Y.”

As used herein and in the appended claims, the singular forms “a,” “an,” and “the” include plural referents unless the context clearly dictates otherwise.

For the recitation of numeric ranges of nucleotides herein, each intervening number therebetween, is explicitly contemplated. For example, for the range of 19-21nt, the number 20nt is contemplated in addition to 19nt and 21nt, and for the range of MOI, each intervening number therebetween, whether it is integral or decimal, is explicitly contemplated.

### Single-Guide RNA^{iBAR} Library

The present application provides one or a plurality of sets of guide RNA constructs and guide RNA libraries comprising guide RNAs (e.g., single-guide RNA) having internal barcodes (iBARs).

In one aspect, the present invention is related to CRISPR/Cas guide RNAs and constructs encoding the CRISPR/Cas guide RNAs. Each guide RNA comprises an iBAR sequence placed in a region of the guide RNA that does not significantly interfere with the interaction between the guide RNA and the Cas nuclease. A plurality (e.g., 2, 3, 4, 5, 6, or more) of sets of guide RNA constructs (including guide RNA molecules and nucleic acids encoding the guide RNA molecules) are provided, in which each guide RNA in a set has the same guide sequence, but a different iBAR sequence. Different sgRNA^{iBAR} constructs of a set having different iBAR sequences can be used in a single gene-editing and screening experiment to provide replicate data.

One aspect of the present application provides a set of sgRNA^{iBAR} constructs comprising three or more (e.g., four) sgRNA^{iBAR} constructs each comprising or encoding an sgRNA^{iBAR} wherein each sgRNA^{iBAR} has an sgRNA^{iBAR} sequence comprising a guide sequence and an iBAR sequence, wherein each guide sequence is complementary to a target genomic locus, wherein the guide sequences for the three or more sgRNA^{iBAR} constructs are the same, wherein the iBAR sequence for each of the three or more sgRNA^{iBAR} constructs is different from each other, and wherein each sgRNA^{iBAR} is operable with a Cas protein to modify the target genomic locus. In some embodiments, each sgRNA^{iBAR} sequence comprises a first stem sequence and a second stem sequence, wherein the first stem sequence hybridizes with the second stem sequence to form a double-stranded RNA region that interacts with the Cas protein, and wherein the iBAR sequence is disposed between the first stem sequence and the second stem sequence. In some embodiments, each sgRNA^{iBAR} sequence comprises in the 5′-to-3′ direction a first stem sequence and a second stem sequence, wherein the first stem sequence hybridizes with the second stem sequence to form a double-stranded RNA region that interacts with the Cas protein, and wherein the iBAR sequence is disposed between the 3′ end of the first stem sequence and the 5′ end of the second stem sequence. In some embodiments, each iBAR sequence comprises about 1-50 nucleotides. In some embodiments, each sgRNA^{iBAR} construct is a plasmid or a viral vector (e.g., lentiviral vector).

In some embodiments, there is provided a set of sgRNA^{iBAR} constructs comprising three or more (e.g., four) sgRNA^{iBAR} constructs each comprising or encoding an sgRNA^{iBAR} wherein each sgRNA^{iBAR} has an sgRNA^{iBAR} sequence comprising a guide sequence and an iBAR sequence, wherein each guide sequence is complementary to a target genomic locus, wherein the guide sequences for the three or more sgRNA^{iBAR} constructs are the same, wherein the iBAR sequence for each of the three or more sgRNA^{iBAR} constructs is different from each other, and wherein each sgRNA^{iBAR} is operable with a Cas9 protein to modify the target genomic locus. In some embodiments, each sgRNA^{iBAR} sequence comprises a guide sequence fused to a second sequence, wherein the second sequence comprises a repeat-anti-repeat stem loop that interacts with the Cas9. In some embodiments, the second sequence of each sgRNA^{iBAR} sequence further comprises a stem loop 1, stem loop 2, and/or stem loop 3. In some embodiments, the iBAR sequence is disposed in the loop region of the repeat-anti-repeat stem loop, and/or the loop region of the stem loop 1, stem loop 2, or stem loop 3. In some embodiments, the iBAR sequence is inserted in the loop region of the repeat-anti-repeat stem loop, and/or the loop region of the stem loop 1, stem loop 2, or stem loop 3. In some embodiments, each iBAR sequence comprises about 1-50 nucleotides. In some embodiments, each sgRNA^{iBAR} construct is a plasmid or a viral vector (e.g., lentiviral vector).

In some embodiments, there is provided a set of sgRNA^{iBAR} constructs comprising three or more (e.g., four) sgRNA^{iBAR} constructs each comprising or encoding an sgRNA^{iBAR} wherein each sgRNA^{iBAR} has an sgRNA^{iBAR} sequence comprising a guide sequence, a second sequence and an iBAR sequence, wherein the guide sequence is fused to a second sequence, wherein the second sequence comprises a repeat-anti-repeat stem loop that interacts with a Cas9 protein, wherein the iBAR sequence is disposed (for example, inserted) in the loop region of the repeat-anti-repeat stem loop, wherein each guide sequence is complementary to a target genomic locus, wherein the guide sequences for the three or more sgRNA^{iBAR} constructs are the same, wherein the iBAR sequence for each of the three or more sgRNA^{iBAR} constructs is different from each other, and wherein each sgRNA^{iBAR} is operable with the Cas9 protein to modify the target genomic locus. In some embodiments, the second sequence of each sgRNA^{iBAR} sequence further comprises a stem loop 1, stem loop 2, and/or stem loop 3. In some embodiments, each iBAR sequence comprises about 1-50 nucleotides. In some embodiments, each sgRNA^{iBAR} construct is a plasmid or a viral vector (e.g., lentiviral vector).

In some embodiments, there is provided a CRISPR/Cas guide RNA construct comprising a guide sequence targeting a genomic locus and a guide hairpin coding for a Repeat:Anti-Repeat Duplex and a tetraloop, wherein an internal barcode (iBAR) is embedded in the tetraloop serving as internal replicates. In some embodiments, the internal barcode (iBAR) comprises a 3 nucleotides (“nt”)-20nt (e.g., 3nt-18nt, 3nt-16nt, 3nt-14nt, 3nt-12nt, 3nt-10nt, 3nt-9nt, 4nt-8nt, 5nt-7nt; preferably, 3nt, 4nt, 5nt, 6nt, 7nt) sequence consisting of A, T, C and G nucleotides. In some embodiments, the guide sequence is 17-23, 18-22, 19-21 nucleotides in length, and the hairpin sequence once transcribed can be bound to a Cas nuclease. In some embodiments, the CRISPR/Cas guide RNA construct further comprises a sequence coding for stem loop 1, stem loop 2 and/or stem loop 3. In some embodiments, the guide sequence targets a genomic gene of a eukaryotic cell, preferably, the eukaryotic cell is a mammalian cell. In some embodiments, the CRISPR/Cas guide RNA construct is a virial vector or a plasmid.

In some embodiments, there is provided an sgRNA^{iBAR} library comprising a plurality of any one of the sets of sgRNA^{iBAR} constructs described herein, wherein each set corresponds to a guide sequence complementary to a different target genomic locus. In some embodiments, the sgRNA^{iBAR} library comprises at least about 1000 sets of sgRNA^{iBAR} constructs. In some embodiments, the iBAR sequences for at least two sets of sgRNA^{iBAR} constructs are the same. In some embodiments, the iBAR sequences for all sets of sgRNA^{iBAR} constructs are the same.

In some embodiments, there is provided an sgRNA^{iBAR} library comprising a plurality of sets of sgRNA^{iBAR} constructs, wherein each set comprises three or more (e.g., four) sgRNA^{iBAR} constructs each comprising or encoding an sgRNA^{iBAR}; wherein each sgRNA^{iBAR} has an sgRNA^{iBAR} sequence comprising a guide sequence and an iBAR sequence, wherein each guide sequence is complementary to a target genomic locus, wherein the guide sequences for the three or more sgRNA^{iBAR} constructs are the same, wherein the iBAR sequence for each of the three or more sgRNA^{iBAR} constructs is different from each other, wherein each sgRNA^{iBAR} is operable with a Cas protein to modify the target genomic locus; and wherein each set corresponds to a guide sequence complementary to a different target genomic locus. In some embodiments, each sgRNA^{iBAR} sequence comprises a first stem sequence and a second stem sequence, wherein the first stem sequence hybridizes with the second stem sequence to form a double-stranded RNA region that interacts with the Cas protein, and wherein the iBAR sequence is disposed between the first stem sequence and the second stem sequence. In some embodiments, each sgRNA^{iBAR} sequence comprises in the 5′-to-3′ direction a first stem sequence and a second stem sequence, wherein the first stem sequence hybridizes with the second stem sequence to form a double-stranded RNA region that interacts with the Cas protein, and wherein the iBAR sequence is disposed between the 3′ end of the first stem sequence and the 5′ end of the second stem sequence. In some embodiments, each iBAR sequence comprises about 1-50 nucleotides. In some embodiments, each sgRNA^{iBAR} construct is a plasmid or a viral vector (e.g., lentiviral vector). In some embodiments, the sgRNA^{iBAR} library comprises at least about 1000 sets of sgRNA^{iBAR} constructs. In some embodiments, the iBAR sequences for at least two sets of sgRNA^{iBAR} constructs are the same.

In some embodiments, there is provided an sgRNA^{iBAR} library comprising a plurality of sets of sgRNA^{iBAR} constructs, wherein each set comprises three or more (e.g., four) sgRNA^{iBAR} constructs each comprising or encoding an sgRNA^{iBAR}; wherein each sgRNA^{iBAR} has an sgRNA^{iBAR} sequence comprising a guide sequence and an iBAR sequence, wherein each guide sequence is complementary to a target genomic locus, wherein the guide sequences for the three or more sgRNA^{iBAR} constructs are the same, wherein the iBAR sequence for each of the three or more sgRNA^{iBAR} constructs is different from each other, wherein each sgRNA^{iBAR} is operable with a Cas9 protein to modify the target genomic locus; and wherein each set corresponds to a guide sequence complementary to a different target genomic locus. In some embodiments, each sgRNA^{iBAR} sequence comprises a guide sequence fused to a second sequence, wherein the second sequence comprises a repeat-anti-repeat stem loop that interacts with the Cas9. In some embodiments, the second sequence of each sgRNA^{iBAR} sequence further comprises a stem loop 1, stem loop 2, and/or stem loop 3. In some embodiments, the iBAR sequence is disposed in the loop region of the repeat-anti-repeat stem loop, and/or the loop region of the stem loop 1, stem loop 2, or stem loop 3. In some embodiments, the iBAR sequence is inserted in the loop region of the repeat-anti-repeat stem loop, and/or the loop region of the stem loop 1, stem loop 2, or stem loop 3. In some embodiments, each iBAR sequence comprises about 1-50 nucleotides. In some embodiments, each sgRNA^{iBAR} construct is a plasmid or a viral vector (e.g., lentiviral vector). In some embodiments, the sgRNA^{iBAR} library comprises at least about 1000 sets of sgRNA^{iBAR} constructs. In some embodiments, the iBAR sequences for at least two sets of sgRNA^{iBAR} constructs are the same.

In some embodiments, there is provided an sgRNA^{iBAR} library comprising a plurality of sets of sgRNA^{iBAR} constructs, wherein each set comprises three or more (e.g., four) sgRNA^{iBAR} constructs each comprising or encoding an sgRNA^{iBAR}; wherein each sgRNA^{iBAR} has an sgRNA^{iBAR} sequence comprising a guide sequence, a second sequence and an iBAR sequence, wherein the guide sequence is fused to a second sequence, wherein the second sequence comprises a repeat-anti-repeat stem loop that interacts with a Cas9 protein, wherein the iBAR sequence is disposed (for example, inserted) in the loop region of the repeat-anti-repeat stem loop, wherein each guide sequence is complementary to a target genomic locus, wherein the guide sequences for the three or more sgRNA^{iBAR} constructs are the same, wherein the iBAR sequence for each of the three or more sgRNA^{iBAR} constructs is different from each other, wherein each sgRNA^{iBAR} is operable with the Cas9 protein to modify the target genomic locus; and wherein each set corresponds to a guide sequence complementary to a different target genomic locus. In some embodiments, each iBAR sequence comprises about 1-50 nucleotides. In some embodiments, each sgRNA^{iBAR} construct is a plasmid or a viral vector (e.g., lentiviral vector). In some embodiments, the sgRNA^{iBAR} library comprises at least about 1000 sets of sgRNA^{iBAR} constructs. In some embodiments, the iBAR sequences for at least two sets of sgRNA^{iBAR} constructs are the same. In some embodiments, the second sequence of each sgRNA^{iBAR} sequence further comprises a stem loop 1, stem loop 2, and/or stem loop 3.

Also provided are sgRNA molecules encoded by any one of the sgRNA^{iBAR} constructs, sets, or libraries described herein. Compositions and kits comprising any one of the sgRNA^{iBAR} constructs, molecules, sets, or libraries are further provided.

In some embodiments, there is provided isolated host cells comprising any one of the sgRNA^{iBAR} constructs, molecules, sets, or libraries described herein. In some embodiments, there is provided a host cell library wherein each host cell comprises one or more sgRNA^{iBAR} constructs from an sgRNA^{iBAR} library described herein. In some embodiments, the host cell comprises or expresses one or more components of the CRISPR/Cas system, such as the Cas protein operable with the sgRNA^{iBAR} constructs. In some embodiments, the Cas protein is Cas9 nuclease.

Also provided herein are methods of preparing an sgRNA^{iBAR} library comprising a plurality of sets of sgRNA^{iBAR} constructs, wherein each set corresponds to one of a plurality of guide sequences each complementary to a different target genomic locus, wherein the method comprises: a) designing three or more sgRNA^{iBAR} constructs for each guide sequence, wherein each sgRNA^{iBAR} construct comprises or encodes an sgRNA^{iBAR} having an sgRNA^{iBAR} sequence comprising the corresponding guide sequence and an iBAR sequence, wherein the iBAR sequence corresponding to each of the three or more sgRNA^{iBAR} constructs is different from each other, and wherein each sgRNA^{iBAR} is operable with a Cas protein to modify the corresponding target genomic locus; and b) synthesizing each sgRNA^{iBAR} construct, thereby producing the sgRNA^{iBAR} library. In some embodiments, the method further comprises designing the plurality of guide sequences.

iBAR Sequences

A set of sgRNA^{iBAR} construct comprises three or more sgRNA^{iBAR} constructs each having a different iBAR sequence. In some embodiments, a set of sgRNA^{iBAR} construct comprises three sgRNA^{iBAR} constructs each having a different iBAR sequence. In some embodiments, a set of sgRNA^{iBAR} construct comprises four sgRNA^{iBAR} constructs each having a different iBAR sequence. In some embodiments, a set of sgRNA^{iBAR} construct comprises five sgRNA^{iBAR} constructs each having a different iBAR sequence. In some embodiments, a set of sgRNA^{iBAR} construct comprises six or more sgRNA^{iBAR} constructs each having a different iBAR sequence.

The iBAR sequences may have any suitable length. In some embodiments, each iBAR sequence is about 1-20 nucleotides (“nt”) in length, such as about any one of 2nt-20 nt, 3nt-18nt, 3nt-16nt, 3nt-14nt, 3nt-12nt, 3nt-10nt, 3nt-9nt, 4nt-8nt, 5nt-7nt. In some embodiments, each iBAR sequence is about 3nt, 4nt, 5nt, 6nt, or 7nt long. In some embodiments, the iBAR sequence in each sgRNA^{iBAR} construct has the same length. In some embodiments, the iBAR sequences of different sgRNA^{iBAR} constructs have different lengths.

The iBAR sequences may have any suitable sequences. In some embodiments, the iBAR sequence is a DNA sequence made of A, T, C and G nucleotides. In some embodiments, the iBAR sequence is an RNA sequence made of A, U, C and G nucleotides. In some embodiments, the iBAR sequence has non-conventional or modified nucleotides other than A, T/U, C and G. In some embodiments, each iBAR sequence is 6 nucleotides long consisting of A, T, C and G nucleotides.

In some embodiments, the set of iBAR sequences associated with each set of sgRNA^{iBAR} constructs in a library is different from each other. In some embodiments, the iBAR sequences for at least two sets of sgRNA^{iBAR} constructs in a library are the same. In some embodiments, the same set of iBAR sequences are used for each set of sgRNA^{iBAR} constructs in a library. It is not necessary to design different iBAR sets for different sets of sgRNA^{iBAR} constructs. A fixed set of iBARs can be used for all sets of sgRNA^{iBAR} constructs in a library, or a plurality of iBAR sequences may be randomly assigned to different sets of sgRNA^{iBAR} constructs in a library. Our iBAR strategy with a streamlined analytic tool (iBAR) would facilitate large-scale CRISPR/Cas screens for biomedical discoveries in various settings.

The iBAR sequence may be disposed (including inserted) to any suitable regions in a guide RNA that does not affect the efficiency of the gRNA in guiding the Cas nuclease (e.g., Cas9) to its target site. The iBAR sequence may be placed at the 3′ end or an internal position in an sgRNA. For example, an sgRNA may comprise various stem loops that interact with the Cas nuclease in a CRISPR complex, and the iBAR sequence may be embedded in the loop region of any one of the stem loops. In some embodiments, each sgRNA^{iBAR} sequence comprises a first stem sequence and a second stem sequence, wherein the first stem sequence hybridizes with the second stem sequence to form a double-stranded RNA region that interacts with the Cas protein, and wherein the iBAR sequence is disposed between the first stem sequence and the second stem sequence. In some embodiments, each sgRNA^{iBAR} sequence comprises in the 5′-to-3′ direction a first stem sequence and a second stem sequence, wherein the first stem sequence hybridizes with the second stem sequence to form a double-stranded RNA region that interacts with the Cas protein, and wherein the iBAR sequence is disposed between the 3′ end of the first stem sequence and the 5′ end of the second stem sequence.

For example, the guide RNA of a CRISPR/Cas9 system may comprise a guide sequence targeting a genomic locus, and a guide hairpin sequence coding for a Repeat:Anti-Repeat Duplex and a tetraloop. In some embodiments, an internal barcode (iBAR) is disposed (including inserted) in the tetraloop serving as internal replicates. In the context of an endogenous CRISPR/Cas9 system, the crRNA hybridizes with the trans-activating crRNA (tracrRNA) to form a crRNA:tracrRNA duplex, which is loaded onto Cas9 to direct the cleavage of cognate DNA sequences bearing appropriate protospacer-adjacent motifs (PAM). An endogenous crRNA sequence can be divided into guide (20 nt) and repeat (12nt) regions, whereas an endogenous tracrRNA sequence can be divided into anti-repeat (14 nt) and three tracrRNA stem loops. In some embodiments, the sgRNA binds the target DNA to form a T-shaped architecture comprising a guide: target heteroduplex, a repeat: anti-repeat duplex, and stem loops 1-3. In some embodiments, the repeat and anti-repeat parts are connected by the tetraloop, and the repeat and anti-repeat form a repeat: anti-repeat duplex, connected with stem loop 1 by a single nucleotide (A51), whereas stem loops 1 and 2 are connected by a 5 nt single-stranded linker (nucleotides 63-67). In some embodiments, the guide sequence (nucleotides 1-20) and target DNA (nucleotides 10-200) form the guide: target heteroduplex via 20 Watson-Crick base pairs, and the repeat (nucleotides 21-32) and the anti-repeat (nucleotides 37-50) form the repeat: anti-repeat duplex via nine Watson-Crick base pairs (U22:A49-A26:U45 and G29:C40-A32:U37). In some embodiments, the tracrRNA tail (nucleotides 68-81 and 82-96) forms stem loops 2 and 3 via four and six Watson-Crick base pairs (A69:U80-U72:A77 and G82:C96-G87:C91), respectively. Nishimasu et al. describes a crystal structure of an exemplary CRISPR/Cas9 system (Nishimasu H, et al. Crystal structure of cas9 in complex with guide RNA and target DNA. Cell. 2014; 156:935-949.), which is incorporated into this application in its entirety as reference.

In some embodiments, the iBAR sequence is disposed in the tetraloop, or the loop region of the repeat: anti-repeat stem loop of an sgRNA. In some embodiments, the iBAR sequence is inserted in the tetraloop, or the loop region of the repeat: anti-repeat stem loop of an sgRNA. The tetraloop of the Cas9 sgRNA scaffold is outside the Cas9-sgRNA ribonucleoprotein complex, which has been subject to alterations for various purposes without affecting the activity of its upstream guide sequence.^{9,12} Inventors of the present application have demonstrated that a 6-nt-long iBAR (iBAR₆) may be embedded in the tetraloop of a typical Cas9 sgRNA scaffold without affecting the gene editing efficiency of the sgRNA or increasing off-target effects.

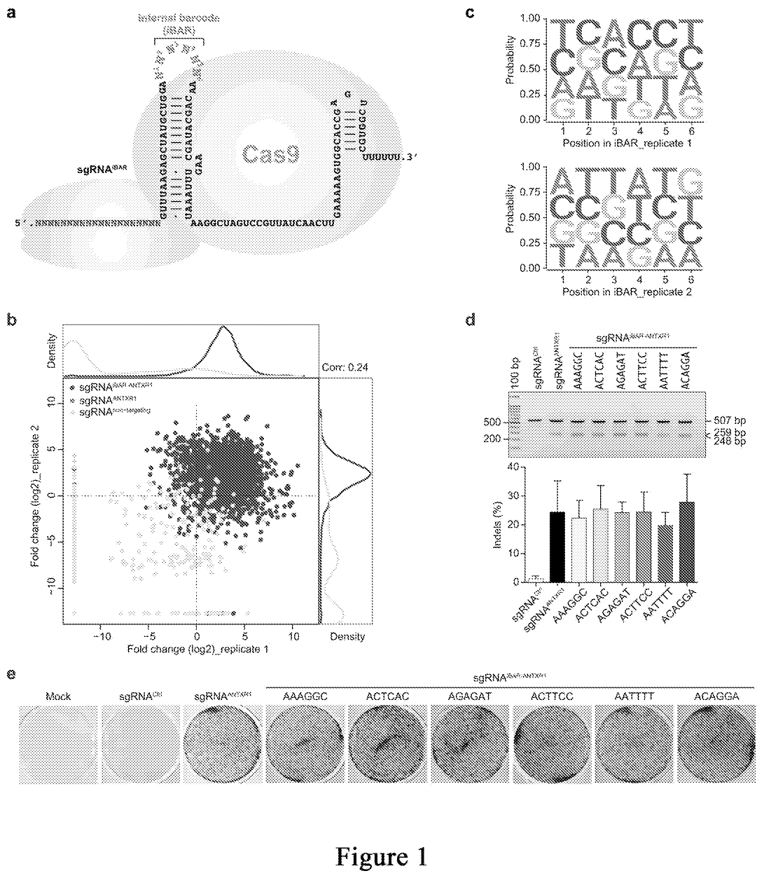

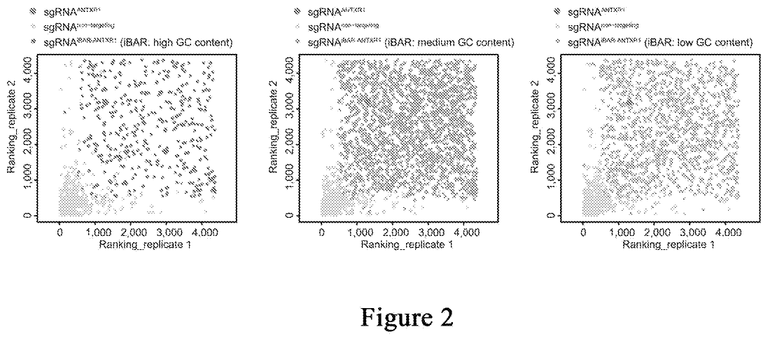

The exemplary iBAR₆ gives rise to 4,096 barcode combinations, which provides sufficient variations for a high throughput screen (FIG. 1A). To determine whether the insertions of these extra iBAR sequences affected the gRNA activities, a library of a pre-determined sgRNA was constructed targeting the anthrax toxin receptor gene ANTXR1¹³ in combination with each of the 4,096 iBAR₆ sequences. This sgRNA^{iBAR-ANTXR1} library was introduced into HeLa cells that constantly express Cas9^{6,7} via lentiviral transduction at a low MOI of 0.3. After three rounds of PA/LFnDTA toxin treatment and enrichment, the sgRNA along with its iBAR₆ sequences from toxin-resistant cells were examined through NGS analysis as previously reported.⁶ The majority of sgRNAs^{iBAR-ANTXR1} and the sgRNAs^{ANTXR1} without barcodes were significantly enriched, whereas almost all the non-targeting control sgRNAs were absent in the resistant cell populations. Importantly, the enrichment levels of sgRNAs^{iBAR-ANTXR1} with different iBAR₆s appeared to be random between two biological replicates (FIG. 1B). After calculating the nucleotide frequency at each position of iBAR₆, no sequence bias was observed from either of the replicates (FIG. 1C). Additionally, the GC contents in iBAR₆ did not seem to affect the sgRNA cutting efficiency (FIG. 2).

### Guide Sequence

The guide sequence hybridizes with the target sequence and direct sequence-specific binding of a CRISPR complex to the target sequence. In some embodiments, the degree of complementarity between a guide sequence and its corresponding target sequence, when optimally aligned using a suitable alignment algorithm, is about or more than about 75%, 80%, 85%, 90%, 91%, 92%, 93%, 94%, 95%, 96%, 97%, 98%, 99%, or more. Optimal alignment may be determined with the use of any suitable algorithm for aligning sequences, non-limiting example of which include the Smith-Waterman algorithm, the Needleman-Wimsch algorithm, algorithms based on the Burrows-Wheeler Transform. In certain embodiments, a guide sequence is about or more than about 10, 11, 12, 13, 14, 15, 16, 17, 18, 19, 20, 21, 22, 23, 24, 25, 26, 27, 28, 29, 30 or more nucleotides in length. The ability of a guide sequence to direct sequence-specific binding of a CRISPR complex to a target sequence may be assessed by any suitable assay. For example, the components of a CRJSPR system sufficient to form a CRISPR complex, including the guide sequence to be tested, may be provided to a host cell having the corresponding target sequence, such as by transfection with vectors encoding the components of the CRISPR sequence, followed by an assessment of preferential cleavage within the target sequence. Similarly, cleavage of a target polynucleotide sequence may be evaluated in a test tube by providing the target sequence, components of a CRISPR complex, including the guide sequence to be tested and a control guide sequence different from the test guide sequence, and comparing binding or rate of cleavage at the target sequence between the test and control guide sequence reactions.

In some embodiments, a guide sequence can be as short as about 10 nucleotides and as long as about 30 nucleotides. In some embodiments, the guide sequence is about any one of 15, 16, 17, 18, 19, 20, 21, 22, 23 or 24 nucleotides long. Synthetic guide sequences can be about 20 nucleotides long, but can be longer or shorter. By way of example, a guide sequence for a CRISPR/Cas9 system may consist of 20 nucleotides complementary to a target sequence, i.e., the guide sequence may be identical to the 20 nucleotides upstream of the PAM sequence except for the A/U difference between DNA and RNA.

The guide sequence in an sgRNA^{iBAR} construct may be designed according to any known methods in the art. The guide sequence may target the coding region such as an exon or a splicing site, the 5′ untranslated region (UTR) or the 3′ untranslated region (UTR) of a gene of interest. For example, the reading frame of a gene could be disrupted by indels mediated by double-strand breaks (DSB) at a target site of a guide RNA. Alternatively, a guide RNA targeting the 5′ end of a coding sequence may be used to produce gene knockouts with high efficiency. The guide sequence may be designed and optimized according to certain sequence features for high on-target gene-editing activity and low off-target effects. For instance, the GC content of a guide sequence may be in the range of 20%-70%, and sequences containing homopolymer stretches (e.g., TTTT, GGGG) may be avoided.

The guide sequence may be designed to target any genomic locus of interest. In some embodiments, the guide sequence targets a genomic locus of a eukaryotic cell, such as a mammalian cell. In some embodiments, the guide sequence targets a genomic locus of a plant cell. In some embodiments, the guide sequence targets a genomic locus of a bacterial cell or an archaeal cell. In some embodiments, the guide sequence targets a protein-coding gene. In some embodiments, the guide sequence targets a gene encoding an RNA, such as a small RNA (e.g., microRNA, piRNA, siRNA, snoRNA, tRNA, rRNA and snRNA), a ribosomal RNA, or a long non-coding RNA (lincRNA). In some embodiments, the guide sequence targets a non-coding region of the genome. In some embodiments, the guide sequence targets a chromosomal locus. In some embodiments, the guide sequence targets an extrachromosomal locus. In some embodiments, the guide sequence targets a mitochondrial or chloroplast gene.

In some embodiments, the guide sequence is designed to repress or activate the expression of any target gene of interest. The target gene may be an endogenous gene or a transgene. In some embodiments, the target gene may be a known to be associated with a particular phenotype. In some embodiments, the target gene is a gene that has not been implicated in a particular phenotype, such as a known gene that is not known to be associated with a particular phenotype or an unknown gene that has not been characterized. In some embodiments, the target region is located on a different chromosome as the target gene.

Other sgRNA Components

The sgRNA^{iBAR} comprises additional sequence element(s) that promote formation of the CRISPR complex with the Cas protein. In some embodiments, the sgRNA^{iBAR} comprises a second sequence comprising a repeat-anti-repeat stem loop. A repeat-anti-repeat stem loop comprises a tracr mate sequence fused to a tracr sequence that is complementary to the tracr mate sequence via a loop region.

Typically, in the context of an endogenous CRISPR/Cas9 system, formation of a CRISPR complex (comprising a guide sequence hybridized to a target sequence and complexed with one or more Cas proteins) results in cleavage of one or both strands in or near (e.g., within 1, 2, 3, 4, 5, 6, 7, 8, 9, 10, 20, 50, or more base pairs from) the target sequence. The tracr sequence, which may comprise or consist of all or a portion of a wild-type tracr sequence (e.g., about or more than about 20, 26, 32, 45, 48, 54, 63, 67, 85, or more nucleotides of a wild-type tracr sequence), may also form part of a CRISPR complex, such as by hybridization along at least a portion of the tracr sequence to all or a portion of a tracr mate sequence that is operably linked to the guide sequence. In some embodiments, the tracr sequence has sufficient complementarity to a tracr mate sequence to hybridize and participate in formation of a CRISPR complex. As with the target sequence, it is believed that complete complementarity is not needed, provided there is sufficient to be functional. In some embodiments, the tracr sequence has at least 50%, 60%, 70%, 80%, 90%, 95% or 99% of sequence complementarity along the length of the tracr mate sequence when optimally aligned. Determining optimal alignment is within the purview of one of skill in the art. For example, there are publically and commercially available alignment algorithms and programs such as, but not limited to, ClustalW, Smith-Waterman in Matlab, Bowtie, Geneious, Biopython and SeqMan. In some embodiments, the tracr sequence is about or more than about 5, 6, 7, 8, 9, 10, 11, 12, 13, 14, 15, 16, 17, 18, 19, 20, 25, 30, 40, 50, or more nucleotides in length. Any one of the known tracr mate sequences and tracr sequences derived from naturally occurring CRISPR system, such as the tracr mate sequence and tracr sequence from the S. pyogenes CRISPR/Cas9 system as described in U.S. Pat. No. 8,697,359 and those described herein, may be used.

In some embodiments, the tracr sequence and tracr mate sequence are contained within a single transcript, such that hybridization between the two produces a transcript having a secondary structure, such as a stem loop (also known as a hairpin), known as the “repeat-anti-repeat stem loop.”

In some embodiments, the loop region of the stem loop in an sgRNA construct without an iBAR sequence is four nucleotides in length, and such loop region is also referred to as the “tetraloop.” In some embodiments, the loop region has the sequence GAAA. However, longer or shorter loop sequences may be used, as may alternative sequences, such as sequences including a nucleotide triplet (for example, AAA), and an additional nucleotide (for example C or G). In some embodiments, the sequence of the loop region is CAAA or AAAG. In some embodiments, the iBAR is disposed in the loop region, such as the tetraloop. In some embodiments, the iBAR is inserted in the loop region, such as the tetraloop. For example, the iBAR sequence may be inserted before the first nucleotide, between the first nucleotide and the second nucleotide, between the second nucleotide and the third nucleotide, between the third nucleotide and the fourth nucleotide, or after the fourth nucleotide in the tetraloop. In some embodiments, the iBAR sequence replaces one or more nucleotides in the loop region.

In some embodiments, the sgRNA^{iBAR} comprises at least two or more stem loops. In some embodiments, the sgRNA^{iBAR} has two, three, four or five stem loops. In some embodiments, the sgRNA^{iBAR} has at most five hairpins. In some embodiments, the sgRNA^{iBAR} construct further includes a transcription termination sequence, such as a polyT sequence, for example six T nucleotides.

In some embodiments, wherein the Cas protein is Cas9, each sgRNA^{iBAR} comprises a guide sequence fused to a second sequence comprising a repeat-anti-repeat stem loop that interacts with the Cas 9. In some embodiments, the iBAR sequence is disposed in the loop region of the repeat-anti-repeat stem loop. In some embodiments, the iBAR sequence is inserted in the loop region of the repeat-anti-repeat stem loop. In some embodiments, the iBAR sequence replaces one or more nucleotides in the loop region of the repeat-anti-repeat stem loop. In some embodiments, the second sequence of each sgRNA^{iBAR} further comprises a stem loop 1, stem loop 2, and/or stem loop 3. In some embodiments, the iBAR sequence is disposed in the loop region of stem loop 1, In some embodiments, the iBAR sequence is inserted in the loop region of stem loop 1. In some embodiments, the iBAR sequence replaces one or more nucleotides in the loop region of stem loop 1. In some embodiments, the iBAR sequence is disposed in the loop region of stem loop 2, In some embodiments, the iBAR sequence is inserted in the loop region of stem loop 2. In some embodiments, the iBAR sequence replaces one or more nucleotides in the loop region of stem loop 2. In some embodiments, the iBAR sequence is disposed in the loop region of stem loop 3, In some embodiments, the iBAR sequence is inserted in the loop region of stem loop 3. In some embodiments, the iBAR sequence replaces one or more nucleotides in the loop region of stem loop 3.

In some embodiments, each sgRNA^{iBAR} sequence comprises a first stem sequence and a second stem sequence, wherein the first stem sequence hybridizes with the second stem sequence to form a double-stranded RNA region that interacts with the Cas protein, and wherein the iBAR sequence is disposed between the first stem sequence and the second stem sequence. In some embodiment, each sgRNA^{iBAR} comprises in the 5′-to-3′ direction a first stem sequence and a second stem sequence, wherein the first stem sequence hybridizes with the second stem sequence to form a double-stranded RNA region that interacts with the Cas protein, and wherein the iBAR sequence is disposed between the 3′ end of the first stem sequence and the 5′ end of the second stem sequence.

In a CRISPR/Cas9 system, a guide RNA can be used to guide the cleavage of a genomic DNA by the Cas9 nuclease. For example, the guide RNA may be composed of a nucleotide spacer of variable sequence (guide sequence) that targets the CRISPR/Cas system nuclease to a genomic location in a sequence-specific manner, and an invariant hairpin sequence that is constant among different guide RNAs and allows the guide RNA to bind to the Cas nuclease. In some embodiments, there is provided a CRISPR/Cas guide RNA comprising a CRISPR/Cas variable guide sequence that is homologous or complementary to a target genomic sequence in a host cell and an invariant hairpin sequence that when transcribed is capable of binding a Cas nuclease (e.g., Cas9), wherein the hairpin sequence codes for a Repeat:Anti-Repeat Duplex and a tetraloop, and an internal barcode (iBAR) is embedded in the tetraloop region.

The guide sequence for a CRISPR/Cas9 guide RNA can be about 17-23, 18-22, 19-21 nucleotides in length. The guide sequence can target the Cas nuclease to a genomic locus in a sequence-specific manner and can be designed following general principles known in the art. The invariant guide RNA hairpin sequences can be provided according to common knowledge in the art, for example, as disclosed by Nishimasu et al. (Nishimasu H, et al. Crystal structure of cas9 in complex with guide RNA and target DNA. Cell. 2014; 156:935-949). The present application also provides examples of the invariant guide RNA hairpin sequence, but it is to be understood that the invention is not so limited and that other invariant hairpin sequences may be used as long as they are capable of binding to a Cas nuclease once transcribed.

Previous studies showed that, although sgRNA with a 48-nt tracrRNA tail (referred to as sgRNA (+48)) is the minimal region, for the Cas9-catalyzed DNA cleavage in vitro (Jinek et al., 2012), sgRNAs with extended tracrRNA tails, sgRNA(+67) and sgRNA(+85), may improve the Cas9 cleavage activity in vivo (Hsu et al., 2013). In some embodiments, the sgRNA^{iBAR} comprises stem loop 1, stem loop 2 and/or stem loop 3. The stem loop 1, stem loop 2 and/or stem loop 3 regions may improve editing efficiency in a CRISPR/Cas9 system.

### Cas Protein

The sgRNA^{iBAR} constructs described herein may be designed to operate with any one of the naturally-occurring or engineered CRISPR/Cas systems known in the art. In some embodiments, the sgRNA^{iBAR} construct is operable with a Type I CRISPR/Cas system. In some embodiments, the sgRNA^{iBAR} construct is operable with a Type II CRISPR/Cas system. In some embodiments, the sgRNA^{iBAR} construct is operable with a Type III CRISPR/Cas system. Exemplary CRISPR/Cas systems can be found in WO2013176772, WO2014065596, WO2014018423, WO2016011080, U.S. Pat. Nos. 8,697,359, 8,932,814, 10,113,167B2, the disclosures of which are incorporated herein by reference in their entireties for all purposes.

In certain embodiments, the sgRNA^{iBAR} construct is operable with a Cas protein derived from a CRISPR/Cas type I, type II, or type III system, which has an RNA-guided polynucleotide binding and/or nuclease activity. Examples of such Cas proteins are recited in, e.g., WO2014144761 WO2014144592, WO2013176772, US20140273226, and US20140273233, which are incorporated herein by reference in their entireties.

In certain embodiments, the Cas protein is derived from a type II CRISPR-Cas system. In certain embodiments, the Cas protein is or is derived from a Cas9 protein. In certain embodiments, the Cas protein is or is derived from a bacterial Cas9 protein, including those identified in WO2014144761.

In some embodiments, the sgRNA^{iBAR} construct is operable with Cas9 (also known as Csn1 and Csx12), a homolog thereof, or a modified version thereof. In some embodiments, the sgRNA^{iBAR} construct is operable with two or more Cas proteins. In some embodiments, the sgRNA^{iBAR} construct is operable with a Cas9 protein from S. pyogenes or S. pneumoniae. Cas enzymes are known in the art; for example, the amino acid sequence of S. pyogenes Cas9 protein may be found in the SwissProt database under accession number Q99ZW2.

The Cas protein (also referred herein as “Cas nuclease”) provides a desired activity, such as target binding, target nicking or cleaving activity. In certain embodiments, the desired activity is target binding. In certain embodiments, the desired activity is target nicking or target cleaving. In certain embodiments, the desired activity also includes a function provided by a polypeptide that is covalently fused to a Cas protein or a nuclease-deficient Cas protein. Examples of such a desired activity include a transcription regulation activity (either activation or repression), an epigenetic modification activity, or a target visualization/identification activity.

In some embodiments, the sgRNA^{iBAR} construct is operable with a Cas nuclease that cleaves the target sequence, including double-strand cleavage and single-strand cleavage. In some embodiments, the sgRNA^{iBAR} construct is operable with a catalytically inactive Cas (“dCas”). In some embodiments, the sgRNA^{iBAR} construct is operable with a dCas of a CRISPR activation (“CRISPRa”) system, wherein the dCas is fused to a transcriptional activator. In some embodiments, the sgRNA^{iBAR} construct is operable with a dCas of a CRISPR interference (CRISPRi) system. In some embodiments, the dCas is fused to a repressor domain, such as a KRAB domain.

In certain embodiments, the Cas protein is a mutant of a wild type Cas protein (such as Cas9) or a fragment thereof. A Cas9 protein generally has at least two nuclease (e.g., DNase) domains. For example, a Cas9 protein can have a RuvC-like nuclease domain and an HNH-like nuclease domain. The RuvC and HNH domains work together to cut both strands in a target site to make a double-stranded break in the target polynucleotide. (Jinek et al., Science 337: 816-21). In certain embodiments, a mutant Cas9 protein is modified to contain only one functional nuclease domain (either a RuvC-like or an HNH-like nuclease domain). For example, in certain embodiments, the mutant Cas9 protein is modified such that one of the nuclease domains is deleted or mutated such that it is no longer functional (i.e., the nuclease activity is absent). In some embodiments where one of the nuclease domains is inactive, the mutant is able to introduce a nick into a double-stranded polynucleotide (such protein is termed a “nickase”) but not able to cleave the double-stranded polynucleotide. In certain embodiments, the Cas protein is modified to increase nucleic acid binding affinity and/or specificity, alter an enzymatic activity, and/or change another property of the protein. In certain embodiments, the Cas protein is truncated or modified to optimize the activity of the effector domain. In certain embodiments, both the RuvC-like nuclease domain and the HNH-like nuclease domain are modified or eliminated such that the mutant Cas9 protein is unable to nick or cleave the target polynucleotide. In certain embodiments, a Cas9 protein that lacks some or all nuclease activity relative to a wild-type counterpart, nevertheless, maintains target recognition activity to a greater or lesser extent.

In certain embodiments, the Cas protein is a fusion protein comprising a naturally-occurring Cas or a variant thereof fused to another polypeptide or an effector domain. The another polypeptide or effector domain may be, for example, a cleavage domain, a transcriptional activation domain, a transcriptional repressor domain, or an epigenetic modification domain. In certain embodiments, the fusion protein comprises a modified or mutated Cas protein in which all the nuclease domains have been inactivated or deleted. In certain embodiments, the RuvC and/or HNH domains of the Cas protein are modified or mutated such that they no longer possess nuclease activity.

In certain embodiments, the effector domain of the fusion protein is a cleavage domain obtained from any endonuclease or exonuclease with desirable properties.

In certain embodiments, the effector domain of the fusion protein is a transcriptional activation domain. In general, a transcriptional activation domain interacts with transcriptional control elements and/or transcriptional regulatory proteins (i.e., transcription factors, RNA polymerases, etc.) to increase and/or activate transcription of a gene. In certain embodiments, the transcriptional activation domain is a herpes simplex virus VP16 activation domain, VP64 (which is a tetrameric derivative of VP16), a NFxB p65 activation domain, p53 activation domains 1 and 2, a CREB (cAMP response element binding protein) activation domain, an E2A activation domain, or an NFAT (nuclear factor of activated T-cells) activation domain. In certain embodiments, the transcriptional activation domain is Ga14, Gcn4, MLL, Rtg3, Gln3, Oaf1, Pip2, Pdr1, Pdr3, Pho4, or Leu3. The transcriptional activation domain may be wild type, or modified or truncated version of the original transcriptional activation domain.

In certain embodiments, the effector domain of the fusion protein is a transcriptional repressor domain, such as inducible cAMP early repressor (ICER) domains, Kruppel-associated box A (KRAB-A) repressor domains, YY1 glycine rich repressor domains, Sp1-like repressors, E(spI) repressors, I. kappa. B repressor, or MeCP2.

In certain embodiments, the effector domain of the fusion protein is an epigenetic modification domain which alters gene expression by modifying the histone structure and/or chromosomal structure, such as a histone acetyltransferase domain, a histone deacetylase domain, a histone methyltransferase domain, a histone demethylase domain, a DNA methyltransferase domain, or a DNA demethylase domain.

In certain embodiments, the Cas protein further comprises at least one additional domain, such as a nuclear localization signal (NLS), a cell-penetrating or translocation domain, and a marker domain (e.g., a fluorescent protein marker).

### Vector

In some embodiments, the sgRNAi^{BAR} construct comprises one or more regulatory elements operably linked to the guide RNA sequence and the iBAR sequence. Exemplary regulatory elements include, but are not limited to, promoters, enhancers, internal ribosomal entry sites (IRES), and other expression control elements (e.g. transcription termination signals, such as polyadenylation signals and poly-U sequences). Such regulatory elements are described, for example, in Goeddel, GENE EXPRESSION TECHNOLOGY: METHODS IN ENZYMOLOGY 185, Academic Press, San Diego, Calif. (1990). Regulatory elements include those that direct constitutive expression of a nucleotide sequence in many types of host cell and those that direct expression of the nucleotide sequence only in certain host cells (e.g., tissue-specific regulatory sequences).

The sgRNA^{iBAR} constructs may be present in a vector. In some embodiments, the sgRNA^{iBAR} construct is an expression vector, such as a viral vector or a plasmid. It will be appreciated by those skilled in the art that the design of the expression vector can depend on such factors as the choice of the host cell to be transformed, the level of expression desired, etc. In some embodiments, the sgRNA^{iBAR} construct is a lentiviral vector. In some embodiments, the sgRNA^{iBAR} construct is an adenovirus or an adeno-associated virus. In some embodiments, the vector further comprises a selection marker. In some embodiments, the vector further comprises one or more nucleotide sequences encoding one or more elements of the CRISPR/Cas system, such as a nucleotide sequence encoding a Cas nuclease (e.g., Cas9). In some embodiments, there is provided a vector system comprising one or more vectors encoding nucleotide sequences encoding one or more elements of the CRISPR/Cas system, and a vector comprising any one of the sgRNA^{iBAR} constructs described herein. A vector may include one or more of the following elements: an origin of replication, one or more regulatory sequences (such as, for example, promoters and/or enhancers) that regulate the expression of the polypeptide of interest, and/or one or more selectable marker genes (such as, for example, antibiotic resistance genes, and fluorescent protein-encoding genes).

### Library

The sgRNA^{iBAR} libraries described herein may be designed to target a plurality of genomic loci according to the needs of a genetic screen. In some embodiments, a single set of sgRNA^{iBAR} constructs is designed to target each gene of interest. In some embodiments, a plurality of (e.g., at least 2, 4, 6, 10, 20 or more, such as 4-6) sets of sgRNA^{iBAR} constructs with different guide sequences targeting a single gene of interest may be designed.

In some embodiments, the sgRNA^{iBAR} library comprises at least 10, 20, 50, 100, 200, 500, 1000, 2000, 5000, 10000, 20000, 50000, 100000, or more sets of sgRNA^{iBAR} constructs. In some embodiments, the sgRNA^{iBAR} library target at least 10, 20, 50, 100, 200, 500, 1000, 2000, 5000, 10000, 15000, or more genes in a cell or organism. In some embodiments, the sgRNA^{iBAR} library is a full-genome library for protein-coding genes and/or non-coding RNAs. In some embodiments, the sgRNA^{iBAR} library is a targeted library, which targets selected genes in a signaling pathway or associated with a cellular process. In some embodiments, the sgRNA^{iBAR} library is used for a genome-wide screen associated with a particular modulated phenotype. In some embodiments, the sgRNA^{iBAR} library is used to for a genome-wide screen to identify at least one target gene associated with a particular modulated phenotype. In some embodiments, the sgRNA^{iBAR} library is designed to target a eukaryotic genome, such as a mammalian genome. Exemplary genomes of interest include genomes of a rodent (mouse, rat, hamster, guinea pig), a domesticated animal (e.g., cow, sheep, cat, dog, horse, or rabbit), a non-human primate (e.g., monkey), fish (e.g., zebrafish), non-vertebrate (e.g., Drosophila melanogaster and Caenorhabditis elegans), and human.

The guide sequences of the sgRNA^{iBAR} libraries may be designed using known algorithms that identify CRISPR/Cas target sites in user-defined lists with a high degree of targeting specificity in the human genome (Genomic Target Scan (GT-Scan); see O'Brien et al., Bioinformatics (2014) 30:2673-2675). In some embodiments, 100,000 sgRNA^{iBAR} constructs can be generated on a single array, providing sufficient coverage to comprehensively screen all genes in a human genome. This approach can also be scaled up to enable genome-wide screens by the synthesis of multiple sgRNA^{iBAR} libraries in parallel. The exact number of sgRNA^{iBAR} constructs in an sgRNA^{iBAR} library can depend on whether the screen 1) targets genes or regulatory elements, 2) targets the complete genome, or subgroup of the genomic genes.

In some embodiments, the sgRNA^{iBAR} library is designed to target every PAM sequence overlapping a gene in a genome, wherein the PAM sequence corresponds to the Cas protein. In some embodiments, the sgRNA^{iBAR} library is designed to target a subset of the PAM sequences found in the genome, wherein the PAM sequence corresponds to the Cas protein.

In some embodiments, the sgRNA^{iBAR} library comprises one or more control sgRNA^{iBAR} constructs that do not target any genomic loci in a genome. In some embodiments, sgRNA^{iBAR} constructs that do not target putative genomic genes can be included in an sgRNA^{iBAR} library as negative controls.

The sgRNA^{iBAR} constructs and libraries described herein may be prepared using any known methods of nucleic acid synthesis and/or molecular cloning methods in the art. In some embodiments, the sgRNA^{iBAR} library is synthesized by electrochemical means on arrays (e.g., CustomArray, Twist, Gen9), DNA printing (e.g., Agilent), or solid phase synthesis of individual oligos (e.g., by IDT). The sgRNA^{iBAR} constructs can be amplified by PCR and cloned into an expression vector (e.g., a lentiviral vector). In some embodiments, the lentiviral vector further encodes one or more components of the CRISPR/Cas-based genetic editing system, such as the Cas protein, e.g., Cas9.

### Host Cells

In some embodiments, there is provided a composition comprising host cells comprising any one of the sgRNA^{iBAR} constructs, molecules, sets, or libraries described herein.

In some embodiments, there is provided a method of editing a genomic locus in a host cell, comprising introducing into a host cell a guide RNA construct comprising a guide sequence targeting a genomic gene and a guide hairpin sequence coding for a Repeat:Anti-Repeat Duplex and a tetraloop, wherein an internal barcode (iBAR) is embedded in the tetraloop serving as internal replicates, expressing the guide RNA that targets the genomic gene in the host cell, and thereby editing the targeted genomic gene in the presence of a Cas nuclease.

In some embodiments, there is provided a cell library prepared by transfecting any one of the sgRNA^{iBAR} libraries described herein to a plurality of host cells, wherein the sgRNA^{iBAR} constructs are present in viral vectors (e.g., lentiviral vectors). In some embodiments, the multiplicity of infection (MOI) between the viral vectors and the host cells during the transfection is at least about 1. In some embodiments, the MOI is at least about any one of 1.5, 2, 2.5, 3, 3.5, 4, 4.5, 5, 5.5, 6, 6.5, 7, 7.5, 8, 8.5, 9, 9.5, 10, or higher. In some embodiments, the MOI is about 1, about 1.5, about 2, about 2.5, about 3, about 3.5, about 4, about 4.5, about 5, about 5.5, about 6, about 6.5, about 7, about 7.5, about 8, about 8.5, about 9, about 9.5, or about 10. In some embodiments, the MOI is about any one of 1-10, 1-3, 3-5, 5-10, 2-9, 3-8, 4-6, or 2-5. In some embodiments, the MOI between the viral vectors and the host cells during transfection is less than 1, such as less than 0.8, 0.5, 0.3, or lower. In some embodiments, the MOI is about 0.3 to about 1.

In some embodiments, one or more vectors driving expression of one or more elements of a CRISPR/Cas system are introduced into a host cell such that expression of the elements of the CRISPR system directs formation of a CRISPR complex with a sgRNA^{iBAR} molecule at one or more target sites. In some embodiments, the host cell has been introduced a Cas nuclease or is engineered to stably express CRISPR/Cas nuclease.

In some embodiments, the host cell is a eukaryotic cell. In some embodiments, the host cell is a prokaryotic cell. In some embodiments, the host cell is a cell line, such as a pre-established cell line. The host cells and cell lines may be human cells or cell lines, or they may be non-human, mammalian cells or cell lines. The host cell may be derived from any tissue or organ. In some embodiments, the host cell is a tumor cell. In some embodiments, the host cell is a stem cell or an iPS cell. In some embodiments, the host cell is a neural cell. In some embodiments, the host cell is an immune cell, such as B cell, or T cell. In some embodiments, the host cell is difficult to transfect with a viral vector, such as lentiviral vector, at a low MOI (e.g., lower than 1, 0.5, or 0.3). In some embodiments, the host cell is difficult to edit using a CRISPR/Cas system at low MOI (e.g., lower than 1, 0.5, or 0.3). In some embodiments, the host cell is available at a limited quantity. In some embodiments, the host cell is obtained from a biopsy from an individual, such as from a tumor biopsy.

### Methods of Screening

The present application also provides methods of genetic screens, including high-throughput screens and full-genome screens, using any one of the guide RNA constructs, guide RNA libraries, and cell libraries described herein.

In some embodiments, there is provided a method of screening for a genomic locus that modulates a phenotype of a cell (e.g., a eukaryotic cell, such as a mammalian cell), comprising: a) contacting an initial population of cells expressing a Cas protein with any one of the sgRNA^{iBAR} libraries described herein under a condition that allows introduction of the sgRNA^{iBAR} constructs into the cells to provide a modified population of cells; b) selecting a population of cells having a modulated phenotype from the modified population of cells to provide a selected population of cells; c) obtaining sgRNA^{iBAR} sequences from the selected population of cells; d) ranking the corresponding guide sequences of the sgRNA^{iBAR} sequences based on sequence counts, wherein the ranking comprises adjusting the rank of each guide sequence based on data consistency among the iBAR sequences in the sgRNA^{iBAR} sequences corresponding to the guide sequence; and e) identifying the genomic locus corresponding to a guide sequence ranked above a predetermined threshold level. In some embodiments, wherein each sgRNA^{iBAR} construct is a plasmid or a viral vector (e.g., lentiviral vector), the sgRNA^{iBAR} library is contacted with the initial population of cells at a multiplicity of infection (MOI) of more than about 2 (e.g., at least about 3, 5 or 10). In some embodiments, more than about 95% of the sgRNA^{iBAR} constructs in the sgRNA^{iBAR} library are introduced into the initial population of cells. In some embodiments, the screening is carried out at more than about 1000-fold coverage. In some embodiments, the screening is positive screening. In some embodiments, the screening is negative screening.

In some embodiments, there is provided a method of screening for a genomic locus that modulates a phenotype of a cell (e.g., a eukaryotic cell, such as a mammalian cell), comprising: a) contacting an initial population of cells with i) any one of the sgRNA^{iBAR} libraries described herein; and ii) a Cas component comprising a Cas protein or a nucleic acid encoding the Cas protein under a condition that allows introduction of the sgRNA^{iBAR} constructs and the Cas component into the cells to provide a modified population of cells; b) selecting a population of cells having a modulated phenotype from the modified population of cells to provide a selected population of cells; c) obtaining sgRNA^{iBAR} sequences from the selected population of cells; d) ranking the corresponding guide sequences of the sgRNA^{iBAR} sequences based on sequence counts, wherein the ranking comprises adjusting the rank of each guide sequence based on data consistency among the iBAR sequences in the sgRNA^{iBAR} sequences corresponding to the guide sequence; and e) identifying the genomic locus corresponding to a guide sequence ranked above a predetermined threshold level. In some embodiments, wherein each sgRNA^{iBAR} construct is a plasmid or a viral vector (e.g., lentiviral vector), the sgRNA^{iBAR} library is contacted with the initial population of cells at a multiplicity of infection (MOI) of more than about 2 (e.g., at least about 3, 5 or 10). In some embodiments, more than about 95% of the sgRNA^{iBAR} constructs in the sgRNA^{iBAR} library are introduced into the initial population of cells. In some embodiments, the screening is carried out at more than about 1000-fold coverage. In some embodiments, the screening is positive screening. In some embodiments, the screening is negative screening.

In some embodiments, there is provided a method of screening for a genomic locus that modulates a phenotype of a cell (e.g., a eukaryotic cell, such as a mammalian cell), comprising: a) contacting an initial population of cells expressing a Cas protein with an sgRNA^{iBAR} library under a condition that allows introduction of the sgRNA^{iBAR} constructs into the cells to provide a modified population of cells; wherein the sgRNA^{iBAR} library comprises a plurality of sets of sgRNA^{iBAR} constructs, wherein each set comprises three or more (e.g., four) sgRNA^{iBAR} constructs each comprising or encoding an sgRNA^{iBAR}; wherein each sgRNA^{iBAR} has an sgRNA^{iBAR} sequence comprising a guide sequence and an iBAR sequence, wherein each guide sequence is complementary to a target genomic locus, wherein the guide sequences for the three or more sgRNA^{iBAR} constructs are the same, wherein the iBAR sequence for each of the three or more sgRNA^{iBAR} constructs is different from each other, wherein each sgRNA^{iBAR} is operable with the Cas protein to modify the target genomic locus; and wherein each set corresponds to a guide sequence complementary to a different target genomic locus; b) selecting a population of cells having a modulated phenotype from the modified population of cells to provide a selected population of cells; c) obtaining sgRNA^{iBAR} sequences from the selected population of cells; d) ranking the corresponding guide sequences of the sgRNA^{iBAR} sequences based on sequence counts, wherein the ranking comprises adjusting the rank of each guide sequence based on data consistency among the iBAR sequences in the sgRNA^{iBAR} sequences corresponding to the guide sequence; and e) identifying the genomic locus corresponding to a guide sequence ranked above a predetermined threshold level. In some embodiments, each sgRNA^{iBAR} sequence comprises a first stem sequence and a second stem sequence, wherein the first stem sequence hybridizes with the second stem sequence to form a double-stranded RNA region that interacts with the Cas protein, and wherein the iBAR sequence is disposed between the first stem sequence and the second stem sequence. In some embodiments, each sgRNA^{iBAR} sequence comprises in the 5′-to-3′ direction a first stem sequence and a second stem sequence, wherein the first stem sequence hybridizes with the second stem sequence to form a double-stranded RNA region that interacts with the Cas protein, and wherein the iBAR sequence is disposed between the 3′ end of the first stem sequence and the 5′ end of the second stem sequence. In some embodiments, each iBAR sequence comprises about 1-50 nucleotides. In some embodiments, the Cas protein is Cas9. In some embodiments, each sgRNA^{iBAR} sequence comprises a guide sequence fused to a second sequence, wherein the second sequence comprises a repeat-anti-repeat stem loop that interacts with the Cas9. In some embodiments, the second sequence of each sgRNA^{iBAR} sequence further comprises a stem loop 1, stem loop 2, and/or stem loop 3. In some embodiments, the iBAR sequence is disposed in the loop region of the repeat-anti-repeat stem loop, and/or the loop region of the stem loop 1, stem loop 2, or stem loop 3. In some embodiments, the iBAR sequence is inserted in the loop region of the repeat-anti-repeat stem loop, and/or the loop region of the stem loop 1, stem loop 2, or stem loop 3. In some embodiments, each sgRNA^{iBAR} construct is a plasmid or a viral vector (e.g., lentiviral vector). In some embodiments, the sgRNA^{iBAR} library is contacted with the initial population of cells at a multiplicity of infection (MOI) of more than about 2 (e.g., at least about 3, 5 or 10). In some embodiments, the sgRNA^{iBAR} library comprises at least about 1000 sets of sgRNA^{iBAR} constructs. In some embodiments, the iBAR sequences for at least two sets of sgRNA^{iBAR} constructs are the same. In some embodiments, more than about 95% of the sgRNA^{iBAR} constructs in the sgRNA^{iBAR} library are introduced into the initial population of cells. In some embodiments, the screening is carried out at more than about 1000-fold coverage. In some embodiments, the screening is positive screening. In some embodiments, the screening is negative screening.

In some embodiments, there is provided a method of screening for a genomic locus that modulates a phenotype of a cell (e.g., a eukaryotic cell, such as a mammalian cell), comprising: a) contacting an initial population of cells with i) an sgRNA^{iBAR} library and ii) a Cas component comprising a Cas protein or a nucleic acid encoding the Cas protein under a condition that allows introduction of the sgRNA^{iBAR} constructs into the cells to provide a modified population of cells; wherein the sgRNA^{iBAR} library comprises a plurality of sets of sgRNA^{iBAR} constructs, wherein each set comprises three or more (e.g., four) sgRNA^{iBAR} constructs each comprising or encoding an sgRNA^{iBAR}; wherein each sgRNA^{iBAR} has an sgRNA^{iBAR} sequence comprising a guide sequence and an iBAR sequence, wherein each guide sequence is complementary to a target genomic locus, wherein the guide sequences for the three or more sgRNA^{iBAR} constructs are the same, wherein the iBAR sequence for each of the three or more sgRNA^{iBAR} constructs is different from each other, wherein each sgRNA^{iBAR} is operable with the Cas protein to modify the target genomic locus; and wherein each set corresponds to a guide sequence complementary to a different target genomic locus; b) selecting a population of cells having a modulated phenotype from the modified population of cells to provide a selected population of cells; c) obtaining sgRNA^{iBAR} sequences from the selected population of cells; d) ranking the corresponding guide sequences of the sgRNA^{iBAR} sequences based on sequence counts, wherein the ranking comprises adjusting the rank of each guide sequence based on data consistency among the iBAR sequences in the sgRNA^{iBAR} sequences corresponding to the guide sequence; and e) identifying the genomic locus corresponding to a guide sequence ranked above a predetermined threshold level. In some embodiments, each sgRNA^{iBAR} sequence comprises a first stem sequence and a second stem sequence, wherein the first stem sequence hybridizes with the second stem sequence to form a double-stranded RNA region that interacts with the Cas protein, and wherein the iBAR sequence is disposed between the first stem sequence and the second stem sequence. In some embodiments, each sgRNA^{iBAR} sequence comprises in the 5′-to-3′ direction a first stem sequence and a second stem sequence, wherein the first stem sequence hybridizes with the second stem sequence to form a double-stranded RNA region that interacts with the Cas protein, and wherein the iBAR sequence is disposed between the 3′ end of the first stem sequence and the 5′ end of the second stem sequence. In some embodiments, each iBAR sequence comprises about 1-50 nucleotides. In some embodiments, the Cas protein is Cas9. In some embodiments, each sgRNA^{iBAR} sequence comprises a guide sequence fused to a second sequence, wherein the second sequence comprises a repeat-anti-repeat stem loop that interacts with the Cas9. In some embodiments, the second sequence of each sgRNA^{iBAR} sequence further comprises a stem loop 1, stem loop 2, and/or stem loop 3. In some embodiments, the iBAR sequence is disposed in the loop region of the repeat-anti-repeat stem loop, and/or the loop region of the stem loop 1, stem loop 2, or stem loop 3. In some embodiments, the iBAR sequence is inserted in the loop region of the repeat-anti-repeat stem loop, and/or the loop region of the stem loop 1, stem loop 2, or stem loop 3. In some embodiments, each sgRNA^{iBAR} construct is a plasmid or a viral vector (e.g., lentiviral vector). In some embodiments, the sgRNA^{iBAR} library is contacted with the initial population of cells at a multiplicity of infection (MOI) of more than about 2 (e.g., at least about 3, 5 or 10). In some embodiments, the sgRNA^{iBAR} library comprises at least about 1000 sets of sgRNA^{iBAR} constructs. In some embodiments, the iBAR sequences for at least two sets of sgRNA^{iBAR} constructs are the same. In some embodiments, more than about 95% of the sgRNA^{iBAR} constructs in the sgRNA^{iBAR} library are introduced into the initial population of cells. In some embodiments, the screening is carried out at more than about 1000-fold coverage. In some embodiments, the screening is positive screening. In some embodiments, the screening is negative screening.

In some embodiments, there is provided a method of screening for a genomic locus that modulates a phenotype of a cell (e.g., a eukaryotic cell, such as a mammalian cell), comprising: a) contacting an initial population of cells expressing a Cas9 protein with an sgRNA^{iBAR} library under a condition that allows introduction of the sgRNA^{iBAR} constructs into the cells to provide a modified population of cells; wherein the sgRNA^{iBAR} library comprises a plurality of sets of sgRNA^{iBAR} constructs, wherein each set comprises three or more (e.g., four) sgRNA^{iBAR} constructs each comprising or encoding an sgRNA^{iBAR}; wherein each sgRNA^{iBAR} has an sgRNA^{iBAR} sequence comprising a guide sequence, a second sequence and an iBAR sequence, wherein the guide sequence is fused to a second sequence, wherein the second sequence comprises a repeat-anti-repeat stem loop that interacts with the Cas9 protein, wherein the iBAR sequence is disposed (for example, inserted) in the loop region of the repeat-anti-repeat stem loop, wherein each guide sequence is complementary to a target genomic locus, wherein the guide sequences for the three or more sgRNA^{iBAR} constructs are the same, wherein the iBAR sequence for each of the three or more sgRNA^{iBAR} constructs is different from each other, wherein each sgRNA^{iBAR} is operable with the Cas9 protein to modify the target genomic locus; and wherein each set corresponds to a guide sequence complementary to a different target genomic locus; b) selecting a population of cells having a modulated phenotype from the modified population of cells to provide a selected population of cells; c) obtaining sgRNA^{iBAR} sequences from the selected population of cells; d) ranking the corresponding guide sequences of the sgRNA^{iBAR} sequences based on sequence counts, wherein the ranking comprises adjusting the rank of each guide sequence based on data consistency among the iBAR sequences in the sgRNA^{iBAR} sequences corresponding to the guide sequence; and e) identifying the genomic locus corresponding to a guide sequence ranked above a predetermined threshold level. In some embodiments, each iBAR sequence comprises about 1-50 nucleotides. In some embodiments, the second sequence of each sgRNA^{iBAR} sequence further comprises a stem loop 1, stem loop 2, and/or stem loop 3. In some embodiments, each sgRNA^{iBAR} construct is a plasmid or a viral vector (e.g., lentiviral vector). In some embodiments, the sgRNA^{iBAR} library is contacted with the initial population of cells at a multiplicity of infection (MOI) of more than about 2 (e.g., at least about 3, 5 or 10). In some embodiments, the sgRNA^{iBAR} library comprises at least about 1000 sets of sgRNA^{iBAR} constructs. In some embodiments, the iBAR sequences for at least two sets of sgRNA^{iBAR} constructs are the same. In some embodiments, more than about 95% of the sgRNA^{iBAR} constructs in the sgRNA^{iBAR} library are introduced into the initial population of cells. In some embodiments, the screening is carried out at more than about 1000-fold coverage. In some embodiments, the screening is positive screening. In some embodiments, the screening is negative screening.

In some embodiments, there is provided a method of screening for a genomic locus that modulates a phenotype of a cell (e.g., a eukaryotic cell, such as a mammalian cell), comprising: a) contacting an initial population of cells with i) an sgRNA^{iBAR} library described herein; and ii) a Cas component comprising a Cas9 protein or a nucleic acid encoding the Cas9 protein under a condition that allows introduction of the sgRNA^{iBAR} constructs and the Cas component into the cells to provide a modified population of cells; wherein the sgRNA^{iBAR} library comprises a plurality of sets of sgRNA^{iBAR} constructs, wherein each set comprises three or more (e.g., four) sgRNA^{iBAR} constructs each comprising or encoding an sgRNA^{iBAR}; wherein each sgRNA^{iBAR} has an sgRNA^{iBAR} sequence comprising a guide sequence, a second sequence and an iBAR sequence, wherein the guide sequence is fused to a second sequence, wherein the second sequence comprises a repeat-anti-repeat stem loop that interacts with the Cas9 protein, wherein the iBAR sequence is disposed (for example, inserted) in the loop region of the repeat-anti-repeat stem loop, wherein each guide sequence is complementary to a target genomic locus, wherein the guide sequences for the three or more sgRNA^{iBAR} constructs are the same, wherein the iBAR sequence for each of the three or more sgRNA^{iBAR} constructs is different from each other, wherein each sgRNA^{iBAR} is operable with the Cas9 protein to modify the target genomic locus; and wherein each set corresponds to a guide sequence complementary to a different target genomic locus; b) selecting a population of cells having a modulated phenotype from the modified population of cells to provide a selected population of cells; c) obtaining sgRNA^{iBAR} sequences from the selected population of cells; d) ranking the corresponding guide sequences of the sgRNA^{iBAR} sequences based on sequence counts, wherein the ranking comprises adjusting the rank of each guide sequence based on data consistency among the iBAR sequences in the sgRNA^{iBAR} sequences corresponding to the guide sequence; and e) identifying the genomic locus corresponding to a guide sequence ranked above a predetermined threshold level. In some embodiments, each iBAR sequence comprises about 1-50 nucleotides. In some embodiments, the second sequence of each sgRNA^{iBAR} sequence further comprises a stem loop 1, stem loop 2, and/or stem loop 3. In some embodiments, each sgRNA^{iBAR} construct is a plasmid or a viral vector (e.g., lentiviral vector). In some embodiments, the sgRNA^{iBAR} library is contacted with the initial population of cells at a multiplicity of infection (MOI) of more than about 2 (e.g., at least about 3, 5 or 10). In some embodiments, the sgRNA^{iBAR} library comprises at least about 1000 sets of sgRNA^{iBAR} constructs. In some embodiments, the iBAR sequences for at least two sets of sgRNA^{iBAR} constructs are the same. In some embodiments, more than about 95% of the sgRNA^{iBAR} constructs in the sgRNA^{iBAR} library are introduced into the initial population of cells. In some embodiments, the screening is carried out at more than about 1000-fold coverage. In some embodiments, the screening is positive screening. In some embodiments, the screening is negative screening.

In some embodiments, there is provided a method for minimizing false discovery rate (FDR) of a CRISPR/Cas-based high-throughput genetic screen, comprising introducing multiple guide RNAs embedded internal barcodes into host cells for tracing the performance of each guide RNA multiple times by counting both the guide RNA and the internal barcode (iBAR) nucleotide sequences in a target cell within the same experiment. In preferred embodiments, the barcodes comprise 2nt-20nt (more preferably, 3nt-18nt, 3nt-16nt, 3nt-14nt, 3nt-12nt, 3nt-10nt, 3nt-9nt, 4nt-8nt, 5nt-7nt; even more preferably, 3nt, 4nt, 5nt, 6nt, 7nt) short sequences consisting of A, T, C and G. In preferred embodiments, the barcodes are embedded in the tetraloop region of the guide RNAs. In preferred embodiments, the guide RNA constructs are virial vectors. In preferred embodiments, the virial vectors are lentiviral vectors. In preferred embodiments, the guide RNA constructs are introduced into the target cells in MOI>1 (for example, MOI>1.5, MOI>2, MOI>2.5, MOI>3, MOI>3.5, MOI>4, MOI>4.5, MOI>5, MOI>5.5, MOI>6, MOI>6.5, MOI>7; such as, MOI is about 1, MOI is about 1.5, MOI is about 2, MOI is about 2.5, MOI is about 3, MOI is about 3.5, MOI is about 4 MOI is about 4.5, MOI is about 5, MOI is about 5.5, MOI is about 6, MOI is about 6.5, MOI is about 7).

As a powerful genome-editing tool, the clustered regularly interspaced short palindromic repeats (CRISPR)-clustered regularly interspaced short palindromic repeats-associated protein 9 (Cas9) system has been quickly developed into a large-scale function-based screening strategy in in eukaryotic cells. Comparing with conventional CRISPR/Cas screen methods, the present invention provides a novel genetic screening method by which the false-positive rate (FDR) of screen is significantly reduced and data reproducibility is greatly increased.

Two papers have recently reported methods to generate random barcodes outside the sgRNA body for pooled CRISPR screening^{13,14} Assuming each sgRNA would create both desired loss-of-function (LOF) and non-LOF alleles, calculating all reads of any given sgRNA is unable to accurately assess the importance of its targeting gene in negative screening. Much improved statistical results could be achieved by linking one UMI (unique molecular identifier) with one editing outcome of each sgRNA to enable single-cell lineage tracing so as to lower the false negative rate, or by counting the decreased number of RSLs (random sequence labels) affiliated with sgRNAs to improve screening quality. Different from these two methods, the present invention provides a novel method using sgRNA sets having iBAR sequences to enable pooled screening with CRISPR library made of viral infection at a high MOI, so as to reduce library size and improve data quality.

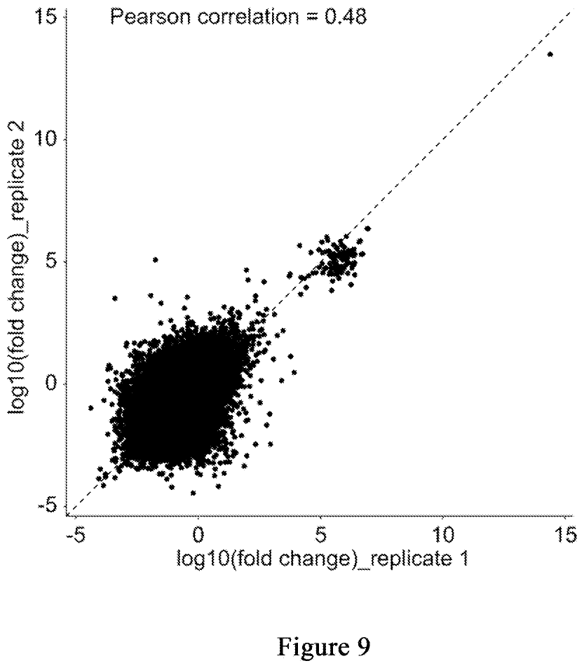

The screening methods described herein use libraries of sets of sgRNA constructs each having internal barcodes (iBARs) in order to improve target identification and data reproducibility by statistical analysis and reduce false discovery rates (FDR). In conventional CRISPR/Cas-based screen methods using a pooled sgRNA library, a high-quality cell library expressing gRNAs are generated using a low multiplicity of infection (MOI) during cell library construction to ensure that each cell harbors on average less than one sgRNA or paired guide RNA (“pgRNA”). Because the sgRNA molecules in a library are randomly integrated in the transfected cells, a sufficiently low MOI ensures that each cell expresses a single sgRNA, thereby minimizing the false-positive rate (FDR) of the screen. To further reduce the FDR and increase data reproducibility, in-depth coverage of gRNAs and multiple biological replicates are often necessary to obtain hit genes with high statistical significance. The conventional screen methods face difficulties when a large number of genome-wide screens are needed, when cell materials for library construction are limited, or when one conducts more challenging screens (i.e., in vivo screen) for which it is difficult to arrange the experimental replications or control the MOL The methods using sgRNA^{iBAR} libraries as described herein overcome the difficulties by including an iBAR sequence in each sgRNA, which enables collection of internal replicates within each sgRNA set having the same guide sequence but different iBAR sequences. For example, an iBAR with four nucleotides for each sgRNA, as described in the Examples, can provide sufficient internal replicates to evaluate data consistency among different sgRNA^{iBAR} constructs targeting the same genomic locus. The high level of consistency between the two independent experiments indicates that one experimental replicate is sufficient for CRISPR/Cas screens using the iBAR method (FIG. 9c and Table 1). Because library coverage is significantly increased with a high MOI during viral transduction of host cells, the cell number in the initial cell population could be reduced more than 20-fold to reach the same library coverage (Table 3), as demonstrated in the constructed genome-wide human library described in the Examples. By the same token, workload for each genome-wide screen using sgRNA^{iBAR} can be reduced proportionally. Using sgRNAs with different iBAR sequences, one could then trace the performance of each guide sequence multiple times within the same experiment by counting both the guide sequence and the corresponding internal barcode (iBAR) nucleotide sequences, thereby drastically reducing FDR, and increasing efficiency and liability. Transduction efficiency and library coverage could be further increased a high viral titer is used during the viral transduction step, for example, with MOI>1 (e.g., MOI>1.5, MOI>2, MOI>2.5, MOI>3, MOI>3.5, MOI>4, MOI>4.5, MOI>5, MOI>5.5, MOI>6, MOI>6.5, MOI>7, MOI>7.5, MOI>8, MOI>8.5, MOI>9, MOI>9.5 or MOI>10; such as, MOI is about 1, MOI is about 1.5, MOI is about 2, MOI is about 2.5, MOI is about 3, MOI is about 3.5, MOI is about 4 MOI is about 4.5, MOI is about 5, MOI is about 5.5, MOI is about 6, MOI is about 6.5, MOI is about 7, MOI is about 7.5, MOI is about 8, MOI is about 8.5, MOI is about 9, MOI is about 9.5, MOI is about 10).

The Cas protein can be introduced into cells in an in vitro or in vivo screen as a (i) Cas protein, or (ii) mRNA encoding the Cas protein, or (iii) a linear or circular DNA encoding the protein. The Cas protein or construct encoding the Cas protein may be purified, or non-purified in a composition. Methods of introducing a protein or nucleic acid construct into a host cell are well known in the art, and are applicable to all methods described herein which requires introduction of a Cas protein or construct thereof to a cell. In certain embodiments, the Cas protein is delivered into a host cell as a protein. In certain embodiments, the Cas protein is constitutively expressed from an mRNA or a DNA in a host cell. In certain embodiments, the expression of Cas protein from mRNA or DNA is inducible or induced in a host cell. In certain embodiments, a Cas protein can be introduced into a host cell in Cas protein: sgRNA complex using recombinant technology known in the art. Exemplary methods of introducing a Cas protein or construct thereof have been described, e.g., in WO2014144761 WO2014144592 and WO2013176772, which are incorporated herein by reference in their entireties.

In some embodiments, the method uses a CRISPR/Cas9 system. Cas9 is a nuclease from the microbial type II CRISPR (clustered regularly interspaced short palindromic repeats) system, which has been shown to cleave DNA when paired with a single-guide RNA (sgRNA). The sgRNA directs Cas9 to complementary regions in the target genome gene, which may result in site-specific double-strand breaks (DSBs) that can be repaired in an error-prone fashion by cellular non-homologous end joining (NHEJ) machinery. Wildtype Cas9 primarily cleaves genomic sites at which the gRNA sequence is followed by a PAM sequence (-NGG). NHEJ-mediated repair of Cas9-induced DSBs induces a wide range of mutations initiated at the cleavage site which are typically small (<10 bp) insertion/deletions (indels) but can include larger (>100 bp) indels.

The methods described herein can be used to identify the functions of coding genes, non-coding RNAs and regulatory elements. In some embodiments, an sgRNA^{iBAR} library is introduced into cells expressing a Cas9 or a catalytically inactive Cas9 (dCas9) fused with an effector domain. By the high-throughput screening, one skilled person in the art can perform multifarious genetic screens by generating diverse mutations, large genomic deletions, transcriptional activation or transcriptional repression. As shown in the Examples, the iBAR sequences do not affect the efficiency of the sgRNAs in guiding the Cas9 or dCas9 nuclease to modify the target sites.

The screening methods described here can be applied to in vitro cell-based screen, or in vivo screens. In some embodiments, the cells are cells in a cell culture. In some embodiments, the cells are present in a tissue or organ. In some embodiments, the cells are present in an organism, such as in C. elegans, flies, or other model organisms.

The initial population of cells can be transduced with a CRISPR/Cas guide RNA library, such as a CRISPR/Cas guide RNA library lentiviral pool. In some embodiments, the sgRNA^{iBAR} viral vector library is introduced to the initial population of cells at a high multiplicity of infection (MOI), such as an MOI of at least about any one of 1, 2, 3, 4, 5, 6, 7, 8, 9 or 10. In some embodiments, the sgRNA^{iBAR} viral vector library is introduced to the initial population of cells at a low MOI, such as an MOI of no more than about any one of 1, 0.9, 0.8, 0.7, 0.6, 0.5, 0.4, 0.3 or lower. In some embodiments, the initial population of cells comprises no more than about any one of 10⁷, 5×10⁶, 2×10⁶, 10⁶, 5×10⁵, 2×10⁵, 10⁵, 5×10⁴, 2×10⁴, 10⁴, or 10³ cells. In some embodiments, more than about any one of 90%, 91%, 92%, 93%, 94%, 95%, 96%, 97%, 98%, 99%, 99.5%, or higher percentage of the sgRNA^{iBAR} constructs in the sgRNA^{iBAR} library are introduced into the initial population of cells. In some embodiments, the screening is carried out at more than about any one of 50-fold, 100-fold, 200-fold, 500-fold, 1000-fold, 2000-fold, 5000-fold, 10,000-fold, or higher folder of coverage.

After introducing the sgRNA^{iBAR} library to the initial population of cells, the cells may be incubated for a suitable period of time to allow gene editing. For example, the cells may be incubated for at least 12 hours, 24 hours, 2 days, 3 days, 4 days, 6 days, 7 days, 8 days, 9 days, 10 days, 11 days, 12 days, 13 days, 14 days, or more. Modified cells having an indel, knock-out, knock-in, activation or repression of target genomic loci or genes of interest are obtained. In some embodiments, transcription of target genes is inhibited or repressed by the sgRNA^{iBAR} constructs in the modified cells. In some embodiments, transcription of target genes is activated by the sgRNA^{iBAR} constructs in the modified cells. In some embodiments, target genes are knocked-out by the sgRNA^{iBAR} constructs in the modified cells. Modified cells may be selected using selectable markers encoded by the sgRNA^{iBAR} vectors, such as fluorescent protein markers or drug-resistance markers.

In some embodiments, the method uses an sgRNA^{iBAR} library designed to target splicing sites or junctions in genes. Splicing-targeting methods can be used to screen a plurality (e.g., thousands) of sequences in the genome, thereby elucidating the function of such sequences. In some embodiments, the splicing-targeting method is used in a high-throughput screen to identify genomic genes required for survival, proliferation, drug resistance, or other phenotypes of interest. In a splicing-targeting experiment, an sgRNA^{iBAR} library targeting tens of thousands of splicing sites within genes of interest may be delivered, for example, by lentiviral vectors, as a pool, into target cells. By identifying sgRNA^{iBAR} sequences that are enriched or depleted in the cells after selection for the desired phenotype, genes that are required for this phenotype can be systematically identified.

In some embodiments, the modified cells are further subject to a stimulus, such as a hormone, a growth factor, an inflammatory cytokine, an anti-inflammatory cytokine, a drug, a toxin, and a transcription factor. In some embodiments, modified cells are treated with a drug to identify genomic loci that increase or decrease sensitivity of the cells to the drug.

In some embodiments, cells with a modulated phenotype are selected from the screen. “Modulate” refers to alteration of an activity, such as regulate, down regulate, upregulate, reduce, inhibit, increase, decrease, deactivate, or activate. Cells with modulated gene expression or cell phenotype can be isolated using known techniques, for example, by fluorescence-activated cell sorting (FACS) or by magnetic-activated cell sorting. The modulated phenotype may be recognized via detection of an intracellular or cell-surface marker. In some embodiments, the intracellular or cell-surface marker can be detected by immunofluorescence staining. In some embodiments, an endogenous target gene can be tagged with a fluorescent reporter, such as by genome editing. Other applicable modulated phenotypic screens include isolating unique cell populations based on a change in response to stimuli, cell death, cell growth, cell proliferation, cell survival, drug resistance, or drug sensitivity.

In some embodiments, the modulated phenotype can be a change in gene expression of at least one target gene or a change in cell or organismal phenotype. In some embodiments, the phenotype is protein expression, RNA expression, protein activity, or RNA activity. In some embodiments, the cell phenotype can be a cell response to stimuli, cell death, cell growth, drug resistance, drug sensitivity, or combinations thereof. The stimuli can be a physical signal, an environmental signal, a hormone, a growth factor, an inflammatory cytokine, an anti-inflammatory cytokine, a transcription factor, a drug or a toxin, or combinations thereof.

In some embodiments, the modified cells are selected for cellular proliferation or survival. In some embodiments, the modified cells are cultured in the presence of a selection agent. The selection agent can be a chemotherapeutic, a cytotoxic agent, a growth factor, a transcription factor, or a drug. In some embodiments, control cells are cultured in the same conditions without the presence of the selection agent. In some embodiments, the selection can be carried out in vivo, e.g., using model organisms. In some embodiments, cells are contacted with the sgRNA^{iBAR} library ex vivo for gene editing, and the gene-edited cells are introduced into an organism (e.g., as xenograft) to select for a modulated phenotype.

In some embodiments, the modified cells are selected for change in expression of one or more genes compared to the expression levels of the one or more genes in control cells. In some embodiments, the change in gene expression is an increase or decrease in gene expression compared to control cells. The change in gene expression can be determined by a change in protein expression, RNA expression, or protein activity. In some embodiments, the change in gene expression occurs in response to a stimulus, such as a chemotherapeutic, a cytotoxic agent, a growth factor, a transcription factor, or a drug.

In some embodiments, control cells are cells that do not comprise sgRNA^{iBAR} constructs, or cells that have been introduced with a negative control sgRNA^{iBAR} construct comprising a guide sequence that does not target any genomic locus in the cells. In some embodiments, control cells are cells that have not been exposed to a stimulus, such as a drug.

The selected population of cells having a modulated phenotype is analyzed by determining sgRNA^{iBAR} sequences in the selected population of cells. The sgRNA^{iBAR} sequences may be obtained by high-throughput sequencing of genomic DNA, RT-PCR, qRT-PCR, RNA-seq or other sequencing methods known in the art. In some embodiments, the sgRNA^{iBAR} sequences are obtained by genome sequencing or RNA sequencing. In some embodiments, the sgRNA^{iBAR} sequences are obtained by next-generation sequencing.

The sequencing data can be analyzed and aligned to the genome using any known methods in the art. In some embodiments, sequence counts of guide RNAs and the corresponding iBAR sequences are determined from the statistical analysis. In some embodiments, the sequence counts are subject to normalization methods, such as median ratio normalization.

Statistical methods may be used to determine the identity of the sgRNA^{iBAR} molecules that are enhanced, or depleted in the selected population of cells. Exemplary statistical methods include, but are not limited to, linear regression, generalized linear regression and hierarchical regression. In some embodiments, the sequence counts are subject to mean-variance modeling following median ratio normalization. In some embodiments, MAGeCK (Li, W. et al. MAGeCK enables robust identification of essential genes from genome-scale CRISPR/Cas9 knockout screens. Genome Biol 15, 554 (2014)) is used to rank guide RNA sequences.

In some embodiments, the variance of each guide sequence is adjusted based on data consistency among the iBAR sequences in the sgRNA^{iBAR} sequences corresponding to the guide sequence. “Data consistency” as used herein refers to consistency of sequencing results of the same guide sequences (e.g., sequence counts, normalized sequence counts, rankings, or fold changes) corresponding to different iBAR sequences in a screening experiment. A true hit from a screen theoretically should have similar normalized sequence counts, rankings, and/or fold changes corresponding to sgRNA^{iBAR} constructs having the same guide sequence, but different iBARs.

In some embodiments, the sequence counts obtained from the selected population of cells are compared to corresponding sequence counts obtained from a population of control cells to provide fold changes. In some embodiments, the data consistency among the iBAR sequences in the sgRNA^{iBAR} sequences corresponding to each guide sequence is determined based on the direction of the fold change of each iBAR sequence, wherein the variance of the guide sequence is increased if the fold changes of the iBAR sequences are in opposite directions with respect to each other. In some embodiments, robust rank aggregation is applied to the sequence counts to determine data consistency.

In a set of sgRNA^{iBAR} constructs, the ranking for the guide sequence may be adjusted based on the consistency of enrichment directions of a pre-determined threshold number m of different iBAR sequences in the set, wherein m is an integer between 1 and n. For example, if at least m iBAR sequences of the sgRNA^{iBAR} set present the same direction of fold change, i.e., all greater or less than that of the control group, then the ranking (or variance) is unchanged. However, if more than n-m different iBAR sequences revealed inconsistent directions of fold change, then the sgRNA^{iBAR} set would be penalized by lowering its ranking, e.g., by increasing its variance. Robust Rank Aggregation (RRA) is one of available tools for statistics and ranking in the art. A skilled person in the art can understand that other tools can also be used for this statistics and ranking. In this invention, Robust Rank Aggregation (RRA) is employed to calculate the final score of each gene in order to obtain the ranking of genes based on mean and variance of every gene. In this way, the sgRNAs whose fold changes among corresponding iBARs are shown in different directions can be penalized through the increased variance leading to lower scores and rankings for certain genes.

In some embodiments, the method is used for positive screening, i.e., by identifying guide sequences that are enhanced in the selected population of cells. In some embodiments, the method is used for negative screening, i.e., by identifying guide sequences that are depleted in the selected population of cells. Guide sequences that are enhanced in the selected population of cells rank high based on sequence counts or fold changes, while guide sequences that are depleted in selected population of cells rank low based on sequence counts or fold changes.

In some embodiments, the method further comprises validating the identified genomic locus. For example, when a genomic locus is identified, experiments using the corresponding sgRNA^{iBAR} constructs may be repeated, or one or more sgRNAs may be designed without iBAR sequences and/or with different guide sequences to target the same gene of interest. Individual sgRNA^{iBAR} or sgRNA constructs may be introduced into the cells to verify the effects of editing the same gene of interest in the cell.

Further provided are methods of analyzing sequencing results from any one of the screening methods described herein. Exemplary methods of analysis are described in the Examples section, including, for example, the MAGeCK^{iBAR} algorithm.

In some embodiments, there is provided a computer system comprising: an input unit that receives a request from a user to identify a genomic locus that modulates a phenotype in a cell; one or more computer processors operatively coupled to the input unit, wherein the one or more computer processors are individually or collectively programmed to: a) receiving a set of sequencing data from a genetic screen using any one of the methods described herein; b) ranking the corresponding guide sequences of the sgRNA^{iBAR} sequences based on sequence counts, wherein the ranking comprises adjusting the rank of each guide sequence based on data consistency among the iBAR sequences in the sgRNA^{iBAR} sequences corresponding to the guide sequence; and c) identifying the genomic locus corresponding to a guide sequence ranked above a predetermined threshold level; and d) presenting the data in a readable manner and/or generating an analysis of the sequencing data.

### Kits and Articles of Manufacture

The present application further provides kits and articles of manufacture for use in any embodiment of the screening methods using the sgRNA^{iBAR} libraries described herein.

In some embodiments, there is provided a kit for screening a genomic locus that modulates a phenotype of a cell, comprising any one of the sgRNA^{iBAR} libraries described herein. In some embodiments, the kit further comprises a Cas protein or a nucleic acid encoding the Cas protein. In some embodiments, the kit further comprises one or more positive and/or negative control sets of sgRNA^{iBAR} constructs. In some embodiments, the kit further comprises data analysis software. In some embodiments, the kit comprises instructions for carrying out any one of the screening methods described herein.

In some embodiments, there is provided a kit for preparing an sgRNA^{iBAR} library useful for a genetic screen, comprising three or more (e.g., four) constructs each comprising a different iBAR sequence and a cloning site for inserting a guide sequence to provide sets of sgRNA^{iBAR} constructs. In some embodiments, the constructs are vectors, such as plasmids or viral vectors (e.g., lentiviral vectors). In some embodiments, the kit comprises instructions for preparing an sgRNA^{iBAR} library and/or for carrying out any one of the screening methods described herein.

The kit may contain additional components, such as containers, reagents, culturing media, primers, buffers, enzymes, and the like to facilitate execution of any one of the screening methods described herein. In some embodiments, the kit comprises reagents, buffers and vectors for introducing the sgRNA^{iBAR} library and the Cas protein or nucleic acid encoding the Cas protein to the cell. In some embodiments, the kit comprises primers, reagents and enzymes (e.g., polymerase) for preparing a sequencing library of sgRNA^{iBAR} sequences extracted from selected cells.

The kits of the present application are in suitable packaging. Suitable packaging includes, but is not limited to, vials, bottles, jars, flexible packaging (e.g., Mylar or plastic bags), and the like. Kits may optionally provide additional components such as buffers and interpretative information. The present application thus also provides articles of manufacture, which include vials (such as sealed vials), bottles, jars, flexible packaging, and the like.

The present application further provides kits or articles of manufacture comprising any of the sgRNA^{iBAR} constructs, sgRNA^{iBAR} molecules, sgRNA^{iBAR} sets, cell libraries, or compositions thereof for use in any one of the screening methods described herein.

### Examples

The examples below are intended to be purely exemplary of the present application and should therefore not be considered to limit the invention in any way. The following examples and detailed description are offered by way of illustration and not by way of limitation.

### Methods

### Cells and Reagents

HeLa and HEK293T cell lines were maintained in Dulbecco's modified Eagle's medium (DMEM, Gibco C11995500BT) supplemented with 1% penicillin/streptomycin and 10% foetal bovine serum (FBS, CellMax BL102-02) and cultured with 5% CO₂ at 37° C. All cells were checked for the absence of mycoplasma contamination.

### Plasmid Construction

The lentiviral sgRNA^{iBAR}_expressing backbone was constructed by changing the position of the BsmBI (Thermo Scientific, ER0451) site using BstBI (NEB, R0519) and XhoI (NEB, R0146) from Plenti-sgRNA-Lib (Addgene, #53121). sgRNA- and sgRNA^{iBAR}-expressing sequences were cloned into the backbone using the BsmBI-mediated Golden Gate cloning strategy²⁸.

Design of the Genome-Scale CRISPR SgRNA^{iBAR} library

Gene annotations were retrieved from the UCSC hg38 genome, which contains 19,210 genes. For each gene, three different sgRNAs that had at least one mismatch in the 16-bp seed region in the genome with a high level of predicted targeting efficiency were designed using our newly developed DeepRank algorithm. We then randomly assigned four 6-bp iBARs (iBAR₆s) to each sgRNA. We designed an additional 1,000 non-targeting sgRNAs, each with four iBAR₆s, to serve as negative controls.

Construction of the CRISPR SgRNA^{iBAR} plasmid library

The 85-nt DNA oligonucleotides were designed and array synthesized. Primers (oligo-F and oligo-R) targeting the flanking sequences of oligos were used for PCR amplification. The PCR products were cloned into the lentiviral vector constructed above using the Golden Gate method²⁸. The ligation mixtures were transformed into Trans 1-T1 competent cells (Transgene, CD501-03) to obtain library plasmids. Transformed clones were counted to ensure at least 100-fold coverage for the scale of the sgRNA^{iBAR} library. The library plasmids were extracted following the standard protocol (QIAGEN 12362) and transfected into HEK293T cells with the two lentivirus package plasmids pVSVG and pR8.74 (Addgene, Inc.) to obtain the library virus. The iBAR library containing all 4,096 iBAR₆s for one ANTXR1-targeting sgRNA was constructed using the same protocol.

Screening of the SgRNA^{iBAR-ANTXR1} library containing all 4,096 types of iBAR₆

A total of 2×10⁷ cells were plated on 150-mm Petri dishes and infected with the library lentivirus at an MOI of 0.3. After 72 h of infection, cells were re-seeded and treated with 1 μg/ml of puromycin (Solarbio P8230) for 48 h. For each replicate, 5×10⁶ cells were collected for genome extraction. Screening of the sgRNA^{iBAR-ANTXR1} library was performed using PA/LFnDTA toxin^{29,30} after library-infected cells were cultured for 15 days. Then, sgRNA with the iBAR coding region in genomic DNA was amplified (TransGen, AP131-13) using Primer-F and Primer-R and then subject to high-throughput sequencing analysis (Illumina HiSeg2500) using an NEBNext Ultra DNA Library Prep Kit for Illumina (NEB E7370L).

### Screening of the Genome-Scale CRISPR/Cas9 SGRNA^{iBAR} Library for Genes Important for TcdB Cytotoxicity and for Genes Essential for Cell Viability

A total of 1.6×10⁸ cells (MOI=0.3), 1.53×10⁷ cells (MOI=3) and 4.6×10⁶ cells (MOI=10) were plated on 150-mm Petri dishes respectively for sgRNA library construction for two replicates. Cells were infected with the library lentivirus of different MOIs and treated with 1 μg/ml of puromycin for 72 h post infection. sgRNA^{iBAR}_integrated cells were cultured for an additional 15 days to maximize gene knock-out. Cells were re-seeded onto 150-mm Petri dishes, treated by TcdB (100 μg/ml) for 10 hrs, and followed by the removal of the loosely attached round cells through repeated pipetting¹⁹. For each round of screening, the cells were cultured in fresh medium without TcdB to reach ˜50%-60% confluence. All resistant cells in one replicate were pooled and subject to another round of TcdB screening. For the subsequent three rounds of screening, the TcdB concentration was 125 μg/ml, 150 μg/ml and 175 μg/ml, respectively. After four rounds of treatment, the resistant cells and untreated cells were collected for genomic DNA extraction, amplification of sgRNA and NGS analysis. 7 pairs of primers were used for PCR amplification (Table 1), and PCR products were mixed for NGS. For negative screening at an MOI of 0.3, a total of 4.6×10⁷ (two replicates) sgRNA^{iBAR}_integrated cells were cultured for 28 days before NGS decoding.

TABLE 1
Primers used for PCR amplification of the genomic DNAs and library construction.

| Name | Sequence | Description |
|---|---|---|
| Oligo- | 5′-TTGTGGAAACGTCTCAACCG (SEQ ID NO: 1) | For PCR |
| F | | amplification of |
| Oligo- | 5′-CTCTAGCTCCGTCTCATGTT (SEQ ID NO: 2) | array-synthesized |
| R | | oligos |
| B-F | 5′-TATATTCGAACGTCTCTAACAGCATAGCAAGTTTAAATA | For construction |
| | AGGCAGTCCGTTATCAACTTGAAAAA (SEQ ID NO: 3) | of the |
| B-R | 5′-TATACTCGAGAAAAAAAAGCACCGACTCGGTGCCACTT | sgRNA^{iBAR}-expressing |
| | TTTCAAGTTGATAACGGACTAGCCTTAT (SEQ ID NO: 4) | backbone |
| AN-F | 5′-AAGCGGAGGACAGGATTGGG (SEQ ID NO: 5) | For PCR |
| AN-R | 5′-CCTCTGTGGCCCTGGAGATG (SEQ ID NO: 6) | amplification of |
| | | the |
| | | sgRNAs^{iBAR-ANTXR1} |
| | | coding region |
| | | for NGS |
| CSPG4- | 5′-CACGGGCCCTTTAAGAAGGT (SEQ ID NO: 7) | For PCR |
| F | | amplification of |
| CSPG4- | 5′-GGACCCACTTCTCACTGTCG (SEQ ID NO: 8) | the T7E1 assay in |
| R | | CSPG4 gene |
| MLH1- | 5′-GTGCTCATCGTTGCCACATATTA (SEQ ID NO: 9) | For PCR |
| F | | amplification of |
| MLH1- | 5′-TACGTGTAACAGACACCTTGC (SEQ ID NO: 10) | the T7E1 assay in |
| R | | MLH1 gene |
| MSH2- | 5′-TTGGGTGTGGTCGCCGTG (SEQ ID NO: 11) | For PCR |
| F | | amplification of |
| MSH2- | 5′-CACAAGCACCAACGTTCCG (SEQ ID NO: 12) | the T7E1 assay in |
| R | | MSH2 gene |
| MSH6- | 5′-TTTTTAAATACTCTTTCCTTGCCTG (SEQ ID NO: 13) | For PCR |
| F | | amplification of |
| MSH6- | 5′-AGGGCGTTTCCTTCCTAGAG (SEQ ID NO: 14) | the T7E1 assay in |
| R | | MSH6 gene |
| PMS2- | 5′-ACACTGTCTTGGGAAATGCAA (SEQ ID NO: 15) | For PCR |
| F1 | | amplification of |
| PMS2- | 5′-TGGCAGCGAGACAAAAC (SEQ ID NO: 16) | the T7E1 assay in |
| R2 | | PMS2 |
| | | gene(sgRNA1,2) |
| PMS2- | 5′-CTCACTGAACACACCATGCC (SEQ ID NO: 17) | For PCR |
| F2 | | amplification of |
| PMS2- | 5′-GGTCTCACTGTGTTGCCCAG (SEQ ID NO: 18) | the T7E1 assay in |
| R2 | | PMS2 |
| | | gene(sgRNA3) |
| 1-F | 5′-TACACGACGCTCTTCCGATCTTAAGTAGAGTATCTTGTG | For PCR |
| | GAAAGGACGAAACACC (SEQ ID NO: 19) | amplification of |
| 1-R | 5′-AGACGTGTGCTCTTCCGATCTTAAGTAGAGAGCTTATCG | the sgRNA^{iBAR} |
| | ATACCGTCGACCTC (SEQ ID NO: 20) | coding region for |
| 2-F | 5′-TACACGACGCTCTTCCGATCTATCATGCTTATATCTTGTG | NGS |
| | GAAAGGACGAAACACC (SEQ ID NO: 21) | |
| 2-R | 5′-AGACGTGTGCTCTTCCGATCTATCATGCTTAAGCTTATC | |
| | GATACCGTCGACCTC (SEQ ID NO: 22) | |
| 3-F | 5′-TACACGACGCTCTTCCGATCTGATGCACATCTTATCTTGT | For PCR |
| | GGAAAGGACGAAACACC (SEQ ID NO: 23) | amplification of |
| 3-R | 5′-AGACGTGTGCTCTTCCGATCTGATGCACATCTAGCTTAT | the sgRNA^{iBAR} |
| | CGATACCGTCGACCTC (SEQ ID NO: 24) | coding region for |
| 4-F | 5′-TACACGACGCTCTTCCGATCTCGATTGCTCGACTATCTT | NGS |
| | GTGGAAAGGACGAAACACC (SEQ ID NO: 25) | |
| 4-R | 5′-AGACGTGTGCTCTTCCGATCTCGATTGCTCGACAGCTTA | |
| | TCGATACCGTCGACCTC (SEQ ID NO: 26) | |
| 5-F | 5′-TACACGACGCTCTTCCGATCTTCGATAGCAATTCTATCTT | |
| | GTGGAAAGGACGAAACACC (SEQ ID NO: 27) | |
| 5-R | 5′-AGACGTGTGCTCTTCCGATCTTCGATAGCAATTCAGCTT | |
| | ATCGATACCGTCGACCTC (SEQ ID NO: 28) | |
| 6-F | 5′-TACACGACGCTCTTCCGATCTATCGATAGTTGCTTTATCT | |
| | TGTGGAAAGGACGAAACACC (SEQ ID NO: 29) | |
| 6-R | 5′-AGACGTGTGCTCTTCCGATCTATCGATAGTTGCTTAGCTT | |
| | ATCGATACCGTCGACCTC (SEQ ID NO: 30) | |
| 7-F | 5′-TACACGACGCTCTTCCGATCTGATCGATCCAGTTAGTAT | |
| | CTTGTGGAAAGGACGAAACACC (SEQ ID NO: 31) | |
| 7-R | 5′-AGACGTGTGCTCTTCCGATCTGATCGATCCAGTTAGAGC | |
| | TTATCGATACCGTCGACCTC (SEQ ID NO: 32) | |

### Screening of the Genome-Scale CRISPR/Cas9 SGRNA^{iBAR} Library for Genes Important for 6-TG Cytotoxicity

A total of 5×10⁷ cells were plated on 150-mm Petri dishes, and two replicates were obtained. Cells were infected with the library lentivirus at an MOI of 3 and treated with 1 μg/ml puromycin 72 h after infection. sgRNA^{iBAR}_integrated cells were cultured for an additional 15 days, re-seeded at a total number of 5×10⁷ and then treated with 200 ng/ml 6-TG (Selleck). For the following two rounds of screening, the 6-TG concentration was 250 ng/ml and 300 ng/ml. For each round of selection, the drug was maintained for 7 days, and the cells were cultured in fresh medium without 6-TG for another 3 days. Then, all the resistant cells in one replicate were grouped together and subject to another round of 6-TG screening. After three rounds of treatment, the resistant cells and untreated cells were collected for genomic DNA extraction, amplification of sgRNA with iBAR regions and deep-sequencing analysis.

### Positive Screening Data Analysis

MAGeCK^{iBAR} is the analysis strategy developed for screens using an sgRNA^{iBAR} library based on MAGeCK algorithm¹⁷. MAGeCK^{iBAR} takes great advantage of Python, Pandas, NumPy, SciPy. The analysis algorithm contains three main parts: analysis preparation, statistical tests and rank aggregation. In the analysis preparation stage, the inputted raw counts of sgRNAs^{iBAR} are normalized, and the coefficients of the population mean and variance are then modelled. In the statistical test stage, we use tests to determine the significance of the difference between the treatment and control normalized reads. In the rank aggregation stage, we aggregate the ranks of all the sgRNAs^{iBAR} targeting each gene to obtain the final gene ranking.

### Normalization and Preparation

We first obtained the raw counts of sgRNAs^{iBAR} from sequencing data. Because the sequencing depth and sequencing error might affect the raw counts of the sgRNAs^{iBAR}, normalization was needed before the following analysis. A size factor was estimated to normalize the raw counts with different sequencing depths. However, because a few highly enriched sgRNAs might have strong influences on the total read counts, the ratio to total read counts should not be used in the normalization. Thus, we chose the median ratio normalization³¹. Suppose there were n sgRNAs in the library, with i ranging from 1 to n, and m experiments in total (both control and treatment groups), with j ranging from 1 to m. The size factor sⱼ can be expressed as follows:

$${s}_{j} = median ( \frac{{k}_{ij}}{{\prod }_{v = 1}^{m} {k}_{iv}^{1 / m}} )$$

Thus, we obtained the normalized counts of sgRNAs^{iBAR} in each experiment by calculating the corresponding size factor. In the mean-variance modelling step, the NB distribution was used to estimate the mean and variance of every sgRNA^{iBAR} across biological replicates and different treatments³²:

Kᵢⱼ˜NB(μᵢⱼ,σᵢⱼ²)

We used the model adopted by MAGeCK to calculate the coefficients of the mean and variance¹⁷. The mean-variance model satisfied the following relationship:

σ⁻²=μ+kμ^{h}

To determine the k and b coefficients from all the sgRNAs^{iBAR} in the library, the function can be transformed into a linear function:

log₂(σ²−μ)=log₂ k+b log₂ μ

The means of the treatment and control counts were calculated directly, and the corresponding variance could be calculated from the mean and coefficients. For CRISPR-iBAR analysis, we evaluated the enrichment of sgRNAs through the performances of different iBARs. We designed four iBARs for each sgRNA to serve as internal replicates. Due to the high MOI during library construction, there must be free riders of false-positive sgRNAs associated with true-positive hits. The free rider here was used to describe the sgRNAs targeting irrelevant genes that were mis-associated with functional sgRNAs to enter the same cells. We modified the variance of sgRNAs^{iBAR} based on the enrichment directions of different iBARs for each sgRNA. If all the iBARs of one sgRNA presented the same direction of fold change, i.e., all greater or less than that of the control group, then the variance would be unchanged. However, if one sgRNA with different iBARs revealed inconsistent directions of fold change, then this kind of sgRNA would be penalized by increasing its variance. The final adjusted variance for inconsistent sgRNAs^{iBAR} would be the model-estimated variance plus the experimental variance calculated from the Ctrl and Exp samples.

Finally, the score of an sgRNA^{iBAR} was calculated by the mean and normalized variance of the treatment compared to those of the control group:

$${score}_{i} = \frac{{t}_{i} - {c}_{i}}{{v}_{i}}$$

where tᵢ is the mean of the treatment counts of the i-th sgRNA, and cᵢ and vᵢ are the mean and variance of control counts of the i-th sgRNA. Because the variance is used as the denominator to calculate score, the enlarged variance for the inconsistent sgRNAs^{iBAR} results in lower score.

### Statistical Test and Rank Aggregation

The normal distribution was used to test the scare, of the treatment counts. The two sides of scores in a standard normal distribution provided the greater-tail and lesser-tail P value separately.

To obtain the gene ranks, we used RRA (robust rank aggregation method), which is an appropriate method for aggregating rankings³³. MAGeCK adopted a modified RRA method by limiting the enriched sgRNAs¹⁷. Suppose for one gene there are n sgRNAs with different iBARs in the library of M sgRNAs^{iBAR} in total; every sgRNA^{iBAR} has a rank in the library of R=(R₁, R₂, . . . , Rₙ). First, the ranks of sgRNAs^{iBAR} should be normalized by the total number of sgRNAs^{iBAR} in the library. We obtained the normalized rank r=(r₁, r₂, . . . , rₙ) for each rᵢ=Rᵢ/M, in which 1≤i≤n. Then, we calculated the sorted normalized ranking s, making sr₁≤sr₂≤ . . . ≤srₙ. The sorted normalized rank follows a uniform distribution between 0 and 1. The probability β_{k,n}(sr) in which srᵢ≤rᵢ follows a β distribution β(k, n+1−k), making ρ=min(β_{1,n}, β_{2,n}, . . . , β_{n,n}). For every gene, the [symbol] score can be obtained by RRA and further adjusted by Bonferroni correction³³. We adopted MAGeCK, which developed α-RRA, to select the top α% sgRNAs from the ranking list. The P values of sgRNAs lower than a threshold (0.25 for instance) were selected. Only the top sgRNAs of one gene were considered in the RRA calculation, thus making ρ=min(β_{1,n}, β_{2,n}, . . . , β_{j,n}), in which 1≤j≤n.

### Negative Screening Data Analysis

During the analyzing process of positive screening at high MOI based on iBAR strategy, we modified the model-estimated variance of sgRNAs with different fold change directions among corresponding barcodes. But for negative screening, most of the non-functional sgRNAs would be unchanged. So the variance modification algorithm based on fold change directions of corresponding barcodes becomes not sufficient to justify whether certain sgRNA is false positive result. Therefore, we treated barcodes as internal replicates directly. When taking iBAR into consideration, we performed two times robust rank aggregation for the negative screening rather than variance adjustment for the inconsistent sgRNAs^{iBAR}. The first round of robust rank aggregation aggregates the sgRNA^{iBAR} level to sgRNA level, and the second round aggregates the sgRNA level to gene level.

### Validation of Candidate Genes

To validate each gene, we chose two sgRNAs designed in the library and cloned into a lentiviral vector with a puromycin selection marker. We mixed two sgRNA plasmids and co-transfected them into HEK293T cells with two lentiviral package plasmids (pVSVG and pR8.74) using the X-tremeGENE HP DNA transfection reagent (Roche). The HeLa cells stably expressing Cas9 were infected with the lentivirus for 3 days and treated with 1 μg/ml puromycin for 2 days. Then, 5,000 cells were added into each well, and five replicates were obtained for each group. After 24 h, the experimental groups were treated with 150 ng/ml 6-TG, and the control groups were treated with normal medium for 7 days. Then, MTT (Amresco) staining and detection were performed following the standard protocol. The experimental wells treated with 6-TG were normalized to the wells without 6-TG treatment.

### Results

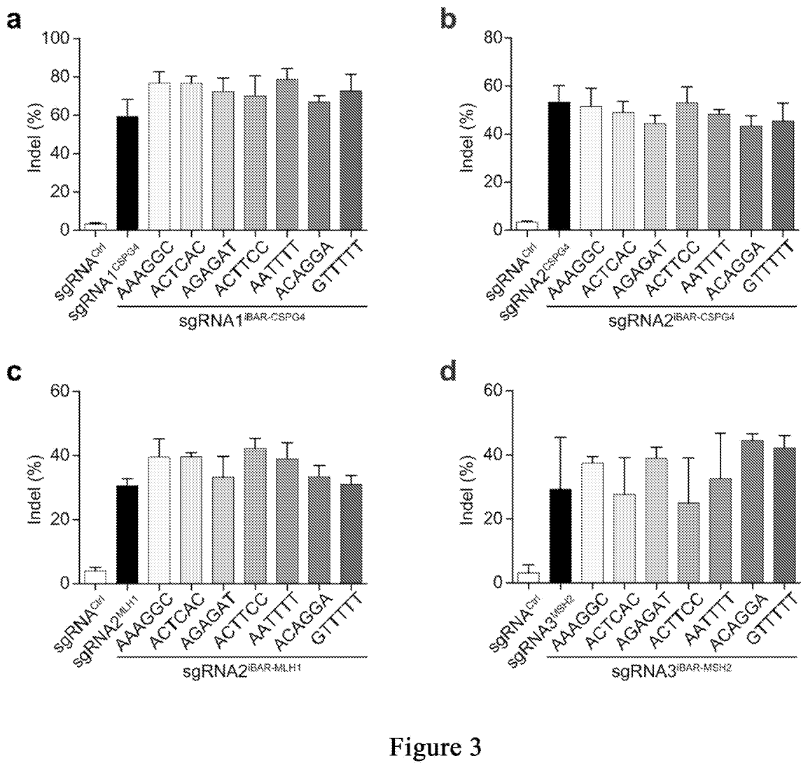

We arbitrarily designed a 6-nt-long iBAR (iBAR₆) that gave rise to 4,096 barcode combinations, providing sufficient variation for our purposes (FIG. 1A). To determine whether the insertions of these extra iBAR sequences affected the gRNA activities, we constructed a library of a pre-determined sgRNA targeting the anthrax toxin receptor gene ANTXR1¹⁶ in combination with all 4,096 types of iBAR₆. This special sgRNA^{iBAR-ANTXR1} library was constructed in HeLa cells that constantly express Cas9^{7,8} through lentiviral transduction at a low MOI of 0.3. After three rounds of PA/LFnDTA toxin treatment and enrichment, the sgRNA along with its iBAR₆ sequences from toxin-resistant cells were examined through NGS analysis as previously reported. The majority of sgRNAs^{iBAR-ANTXR1} and the sgRNAs^{ANTXR1} without barcodes were significantly enriched, whereas almost all the non-targeting control sgRNAs were absent in the resistant cell populations. Importantly, the enrichment levels of sgRNAs^{iBAR-ANTXR1} with different iBAR₆s appeared to be random between two biological replicates (FIG. 1B). After calculating the nucleotide frequency at each position of iBAR₆, we failed to observe any bias of nucleotides from either of the replicates (FIG. 1C). Additionally, the GC contents in iBAR₆ did not seem to affect the sgRNA cutting efficiency (FIG. 2). However, there was a small number of iBAR₆s whose affiliated sgRNA^{ANTXR1} did not perform well in either screening replicate. To rule out the possibility that these iBAR₆s had negative effects on sgRNA activity, we selected six different iBARs from the bottom of the sgRNA^{iBAR-ANTXR1} ranking for further investigation. Compared to the control sgRNA^{ANTXR1} without a barcode, all six of these sgRNAs^{iBAR-ANTXR1} showed comparable efficiency in generating both DNA double-stranded breaks (DSBs) at target sites (FIG. 1D) and ANTXR1 gene disruption leading to the toxin resistance phenotype (FIG. 1E). We further confirmed the negligible effects of iBARs on sgRNA efficiency by four different sgRNAs targeting CSPG4, MLH1 and MSH2, respectively (FIG. 3). Taken together, these results indicate that this re-designed sgRNA^{iBAR} retains sufficient activity of sgRNA, making it possible to generally apply this strategy in CRISPR-pooled screens.

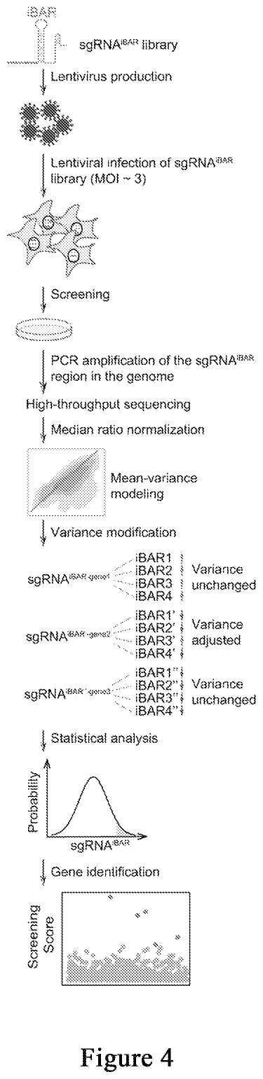

Based on the iBAR strategy, we then set out to broaden its application to perform a novel sgRNA^{iBAR} library screen at a high MOL We followed the standard procedure to harvest the library cells, extract their genomic DNA for PCR amplification of sgRNA with iBAR coding regions and perform NGS analysis^{7,11, 12}. The MAGeCK algorithm could be used to calculate the statistical significance of an sgRNA score through normalization of its raw counts, estimation of its variance using a negative binomial (NB) model and determination of its ranking using a null model with a uniform distribution¹⁷. Taking the iBAR into consideration, we assessed the consistency of any sgRNA count change among all the associated iBARs within the same experimental replicate. This process effectively eliminates free riders that were associated with functional sgRNAs due to lentiviral infection at a high MOI in cell library construction. Specifically, for the iBAR system, we purposely adjusted the model-estimated variance for only those sgRNAs whose fold changes with multiple iBARs were in opposite directions, resulting in increased P-values for these outliers. Finally, we identified hit genes based on sgRNA scores and technical variance between biological replicates (FIG. 4). We developed this specific MAGeCK-based algorithm named MAGeCK^{iBAR} for the analysis of sgRNA^{iBAR} library screening that is open source and freely available for download.

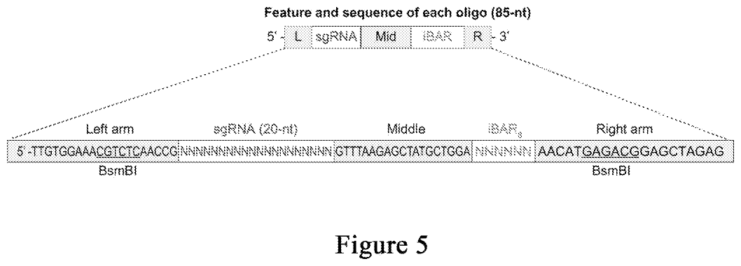

We then constructed an sgRNA^{iBAR} library covering every annotated human gene. For each of the 19,210 human genes, three unique sgRNAs were designed using DeepRank method, each of which was randomly assigned four iBAR₆s. In addition, 1,000 non-targeting sgRNAs, each with four iBAR₆s, were included as negative controls. For the ease of statistical comparison, every set of 3 unique non-targeting sgRNAs was artificially named a negative control gene. The 85-nt sgRNA^{iBAR} oligos were designed in silico (FIG. 5), synthesized using array synthesis, and cloned as a pooled library into a lentiviral backbone. Cas9-expressing HeLa cells were transduced with the sgRNA^{iBAR} library lentivirus at three different MOIs (0.3, 3 and 10) with 400-fold coverage for sgRNAs to generate cell libraries, in which each sgRNA^{iBAR} was covered 100-fold. To evaluate the effect of iBAR design for CRISPR screening at different MOIs, we performed a positive screening to identify genes that mediate the cytotoxicity of Clostridium difficile toxin B (TcdB), one of the key virulence factors of this anaerobic bacillus¹⁸. We have previously reported the first identification of the functional receptor of TcdB, CSPG4¹⁹, whose coding gene was also identified and ranked at the very top from a genome-scale CRISPR library screening²⁰. In this reported CRISPR screening, UGP2 gene was also top-ranked hit, and FZD2 was identified and confirmed to encode the secondary receptor that mediates the TcdB's killing effect on host cells. Of note, the role of FZD2 was significantly dwarfed by CSPG4 so that the FZD2 gene could only be identified using the truncated TcdB that had CSPG4-interacting region deleted²⁰. In our screens on TcdB, we used MAGeCK^{iBAR} and MAGeCK to analyse data from iBAR and the conventional CRISPR screens, respectively. We consequently obtained top-ranked genes (FDR<0.15) from both.

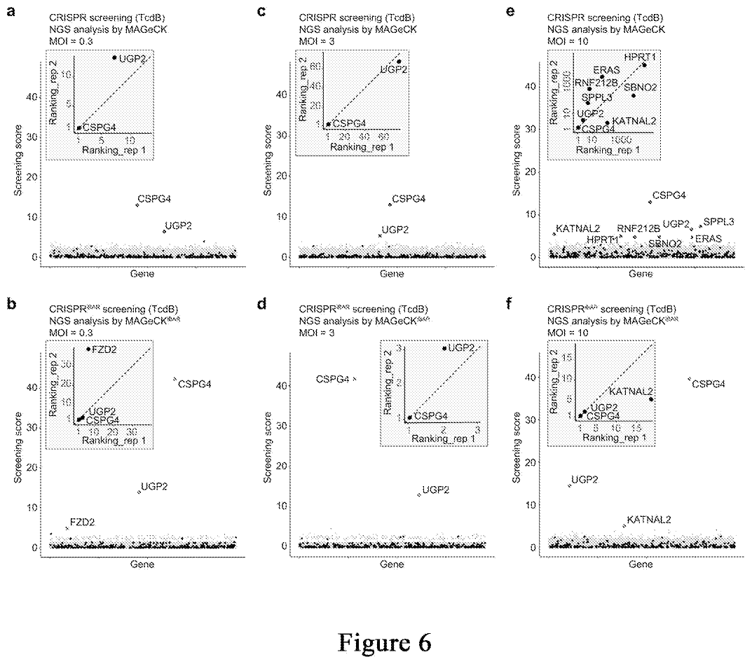

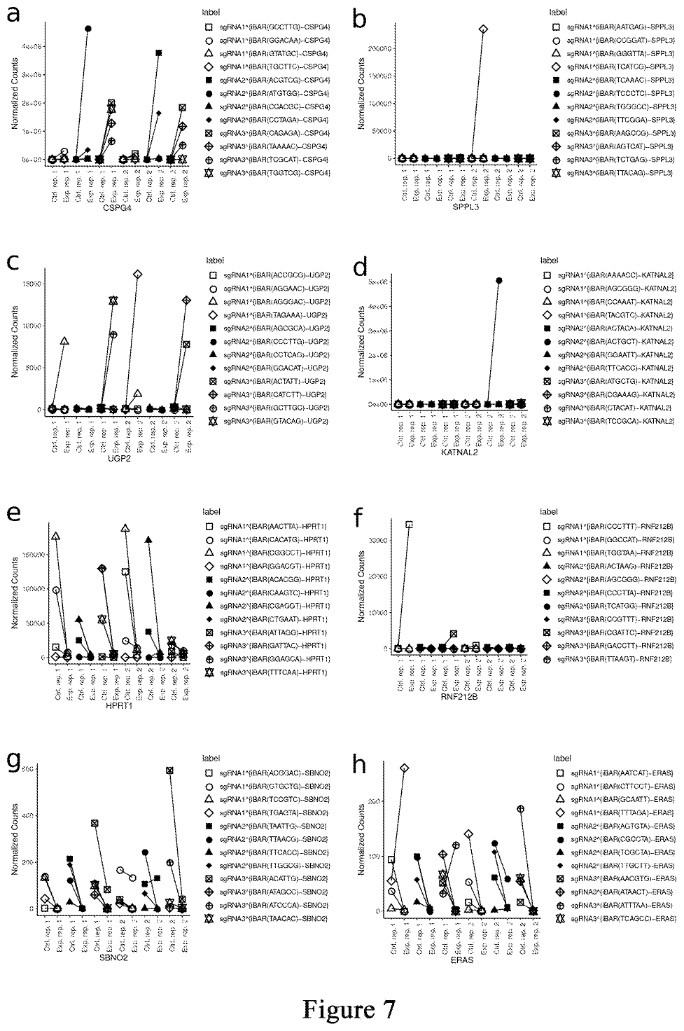

For screening at a low MOI of 0.3, CSPG4 and UGP2 were identified and ranked at the top (FIG. 6A), consistent with the previous report²⁰. When taking iBARs into account, we identified FZD2 in addition to CSPG4 and UGP2 (FIG. 6B). Because FZD2 is a proven receptor of TcdB which plays much weaker role than CSPG4 in HeLa cells²⁰, these results demonstrated that iBAR method offered superior quality and sensitivity to conventional CRISPR screening when constructing cell library at a low MOL In addition, rankings of CSPG4 and UGP2 were far more consistent in CRISPR^{iBAR} screening between two experimental replicates, again indicating the much higher quality for the new method (FIGS. 6A, 6B). At high MOIs (3 and 10), CSPG4 and UGP2 could be isolated from both CRISPR and CRISPR^{iBAR} screens, but the data quality was significantly higher with the latter (FIGS. 6C-6F). In general, the higher the MOI, the worse the signal-to-noise rate for the traditional method. At a MOI of 10, the number of false positive hits was drastically increased in the conventional method, but not in CRISPR^{iBAR} screening (FIGS. 6E, 6F). Impressively, CSPG4 and UGP2 remained top ranked from CRISPR^{iBAR} screening even at an MOI of 10, although the data quality slightly declined (FIG. 6F). Noticeably, nearly all CSPG4- and UGP2-targeting sgRNAs^{iBAR} were significantly enriched after TcdB treatment (FIG. 7), strikingly different from other genes identified at an MOI of 10 using conventional method, such as SPPL3, a likely false positive result (FIG. 7). In comparison of the two biological replicates, CSPG4 and UGP2 were all ranked at the top in both biological replicates from CRISPR^{iBAR} screens with all MOI conditions (FIGS. 6b, 6d, 60, but not from the conventional CRISPR screens where UGP2 was ranked lower than 60^{th} in both replicates at an MOI of 3 (FIG. 6C) and many false positive hits appeared in both replicates at an MOI of 10 (FIG. 6E). These results showed that iBAR method maintained the quality of data even at a high MOI as that at a low MOI for conventional CRISPR screening. Additionally, one biological replicate is likely sufficient to identify hit genes using CRISPR^{iBAR} screening because of the high consistency between two experimental replicates (FIG. 6). After all, multiple replications could be conducted within one experiment based on iBAR approach.

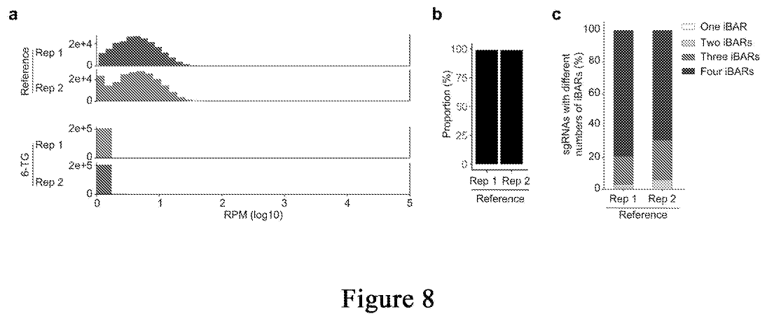

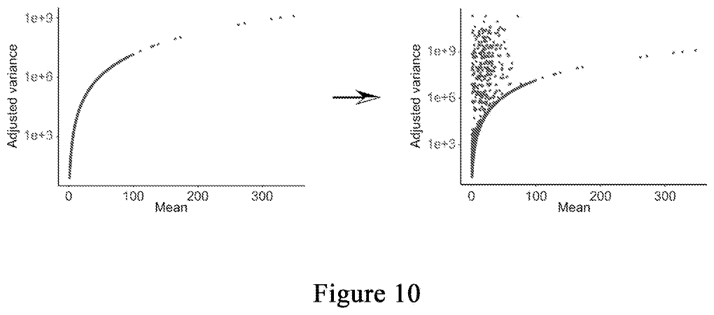

To further evaluate the power of iBAR method, we went on conducting a screening to identify genes that modulate cellular susceptibility to 6-TG²¹, a cancer drug that could be processed to inhibit DNA synthesis. We decided to construct the genome-scale sgRNA^{iBAR} library at a MOI of 3 to generate a cell library with high coverage (2,000-fold) for each sgRNA, in which each sgRNA^{iBAR} was covered 500-fold. The overall read distribution of both experimental replicates was shown (FIG. 8A), and the reference cell libraries of both replicates reached 97% coverage of all originally designed sgRNAs (FIG. 8B). Over 95% of the sgRNAs in the original libraries retained three to four iBARs, indicating the good quality of libraries in which most sgRNAs had sufficient barcode variants for screening and data analysis (FIG. 8C). The fold change of all genes correlated well between the two biological replicates (FIG. 9). For the same 6-TG screening of two sgRNA library replicates, we also employed MAGeCK and MAGeCK^{iBAR} analysis. For MAGeCK^{iBAR}, we consequently obtained adjusted variance and mean distributions for all the sgRNAs^{iBAR} that heightened the variance of sgRNAs with enrichment inconsistent among different iBAR repeats (FIG. 10).

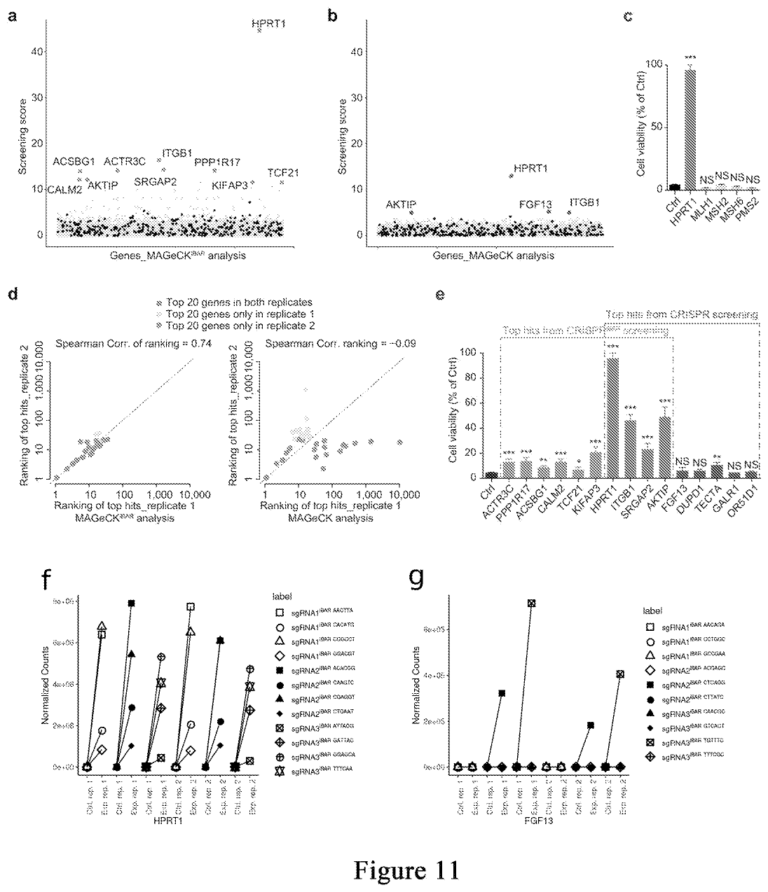

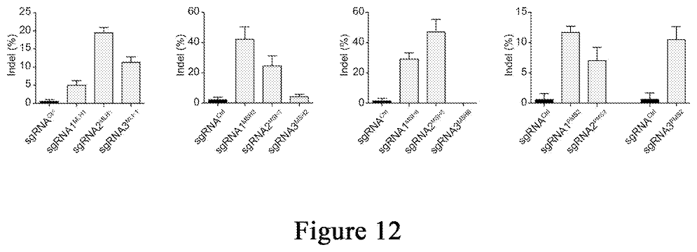

From the positively selected sgRNAs with statistical significance, we identified the top-ranked genes (FDR<0.15) whose corresponding sgRNAs were consistently enriched among different iBARs (FIG. 11A), and we also found these top genes using the MAGeCK algorithm without taking barcodes into account (FIG. 11B). Consistent with a previous report²², the sgRNAs targeting HPRT1 gene were top ranked by both methods. Four genes (MLH1, MSH2, MSH6 and PMS2) were previously reported to be involved in 6-TG-mediated cell death⁶. We examined and confirmed the cutting activities of all except one of the primary designed sgRNAs targeting these four genes (FIG. 12), indicating that these genes were indeed irrelevant to 6-TG-mediated cell death in HeLa cells we used (FIG. 11C). When analysing the two biological replicates separately, the top 20 genes of each replicate showed a high level of consistency with CRISPR^{iBAR} screening (Spearman correlation coefficient for rankings=0.74), whereas the two replicates shared much less commonality when using the conventional method (Spearman correlation coefficient for rankings=−0.09) (FIG. 11D and Table 2).

TABLE 2
Top 20 gene list of two biological replicates using MAGeCKiBAR
and MAGeCK analysis.

| MAGeCK^{iBAR} | | | | MAGeCK | | | |
|---|---|---|---|---|---|---|---|
| Replicate 1 | | Replicate 2 | | Replicate 1 | | Replicate 2 | |
| Gene | Score | Gene | Score | Gene | Score | Gene | Score |
| HPRT1 | 4.29E−33 | HPRT1 | 1.03E−28 | HPRT1 | 1.16E−07 | HPRT1 | 1.75E−06 |
| ITGB1 | 1.28E−17 | ITGB1 | 3.27E−14 | AKTIP | 1.46E−06 | HCRTR2 | 4.25E−06 |
| SRGAP2 | 2.84E−16 | SRGAP2 | 4.68E−14 | ITGB1 | 2.10E−06 | AKTIP | 1.72E−05 |
| ACSBG1 | 3.62E−16 | ACSBG1 | 1.41E−13 | FGF13 | 1.51E−05 | ITGB1 | 2.12E−05 |
| ACTR3C | 4.97E−16 | PPP1R17 | 1.59E−12 | PQLC2L | 3.02E−05 | CXorf51B | 3.02E−05 |
| PPP1R17 | 6.55E−16 | AKTIP | 7.93E−12 | MYL6 | 6.03E−05 | APRT | 6.03E−05 |
| CALM2 | 7.83E−15 | KIFAP3 | 2.68E−11 | C4BPB | 6.46E−05 | FGF13 | 7.11E−05 |
| AUTS2 | 4.50E−14 | CALM2 | 2.94E−11 | CALM2 | 6.52E−05 | EPPK1 | 1.27E−04 |
| FMN2 | 5.66E−14 | TCF21 | 5.73E−11 | AUTS2 | 7.64E−05 | GALR1 | 1.51E−04 |
| AKTIP | 9.30E−14 | ISLR2 | 7.23E−11 | VIT | 9.85E−05 | PQLC2L | 2.11E−04 |
| KIFAP3 | 1.47E−13 | FMN2 | 1.02E−10 | SPSB2 | 1.17E−04 | SAP25 | 2.72E−04 |
| TCF21 | 1.59E−13 | TOR1AIP1 | 3.22E−10 | FMN2 | 1.23E−04 | HSDL1 | 2.94E−04 |
| ISLR2 | 2.75E−12 | CALCRL | 3.82E−10 | CALCRL | 1.29E−04 | LONRF2 | 3.14E−04 |
| OSBPL3 | 3.91E−12 | EVA1B | 5.97E−10 | SRGAP2 | 1.36E−04 | GPAA1 | 3.32E−04 |
| LRRC42 | 4.22E−12 | SH2D1A | 8.27E−10 | ACTR3C | 1.50E−04 | SRR | 3.66E−04 |
| SH2D1A | 4.41E−12 | AUTS2 | 9.84E−10 | GOLM1 | 1.51E−04 | KCNK6 | 3.72E−04 |
| EVA1B | 5.76E−12 | ACTR3C | 3.57E−09 | PPP1R17 | 1.52E−04 | TMPRSS11E | 3.82E−04 |
| FCGR1B | 9.99E−12 | LRRC42 | 5.93E−09 | KIFAP3 | 1.53E−04 | CD93 | 3.92E−04 |
| TOR1AIP1 | 1.47E−11 | ATP6VOC | 7.88E−09 | PPIP5K2 | 1.53E−04 | FMN2 | 4.27E−04 |
| CALCRL | 4.98E−11 | PPIP5K2 | 1.11E−08 | TOR1AIP1 | 1.56E−04 | AUTS2 | 4.28E−04 |

Note:
Genes that ranked in the top 20 list for both replicates are labelled in bold.

To validate the screening results, we de novo designed and combined two sgRNAs to make a mini-pool to target each candidate gene, and each pool was introduced into HeLa cells through lentiviral infection (Table 3).

TABLE 3
sgRNA design for the functional validation of candidate genes
from 6-TG screening and sgRNA design for the test of iBAR
effects on activity

| sgRNA | sequence |
|---|---|
| HPRT1_sgRNA 1 | TCACCACGACGCCAGGGCTG (SEQ ID NO: 33) |
| HPRT1_sgRNA 2 | GTTATGGCGACCCGCAGCCC (SEQ ID NO: 34) |
| ITGB1_sgRNA 1 | ACACAGCAAACTGAACTGAT (SEQ ID NO: 35) |
| ITGB1_sgRNA 2 | TACCTGTTTGAGCAAACACA (SEQ ID NO: 36) |
| SRGAP2_sgRNA 1 | CAGCCAAATTCAAAAAGGAT (SEQ ID NO: 37) |
| SRGAP2_sgRNA 2 | CCAAATTCAAAAAGGATAAG (SEQ ID NO: 38) |
| AKTIP_sgRNA 1 | GCTTGTAGACATGCTCCAGA (SEQ ID NO: 39) |
| AKTIP_sgRNA 2 | CACGTTATGAACCCTTTCTG (SEQ ID NO: 40) |
| ACTR3C_sgRNA 1 | CAGGACTCTACATTGCAGTT (SEQ ID NO: 41) |
| ACTR3C_sgRNA 2 | CGTTCCAGGACTCTACATTG (SEQ ID NO: 42) |
| PPP1R17_sgRNA 1 | TGATGTCCACTGAGCAAATG (SEQ ID NO: 43) |
| PPP1R17_sgRNA 2 | CAGTGGCTGCATTTGCTCAG (SEQ ID NO: 44) |
| ASCBG1_sgRNA 1 | TGGGCAGCCGTATCCAGCTC (SEQ ID NO: 45) |
| ASCBG1_sgRNA 2 | GCAGATGCCACGCAATTCTG (SEQ ID NO: 46) |
| CALM2_sgRNA 1 | GTAGGCTGACCAACTGACTG (SEQ ID NO: 47) |
| CALM2_sgRNA 2 | CAATCTGCTCTTCAGTCAGT (SEQ ID NO: 48) |
| TCF21_sgRNA 1 | ACTCCCCCAAACATGTCCAC (SEQ ID NO: 49) |
| TCF21_sgRNA 2 | CACATCGCTGAGGGAGCCGG (SEQ ID NO: 50) |
| KIFAP3_sgRNA 1 | CAACACAGATATAACTTCCC (SEQ ID NO: 51) |
| KIFAP3_sgRNA 2 | CAGGGAAGTTATATCTGTGT (SEQ ID NO: 52) |
| FGF13_sgRNA 1 | TTGTTCTCTTTGCAGAGCCT (SEQ ID NO: 53) |
| FGF13_sgRNA 2 | TCTTTGCAGAGCCTCAGCTT (SEQ ID NO: 54) |
| DUPD1_sgRNA 1 | CAGATGAGTAGGCATTCTTG (SEQ ID NO: 55) |
| DUPD1_sgRNA 2 | ATGCCTACTCATCTGCCAAG (SEQ ID NO: 56) |
| TECTA_sgRNA 1 | TGAAAGAGACCCAAATTCTA (SEQ ID NO: 57) |
| TECTA_sgRNA 2 | TTCGCACTTGTACAGCACCA (SEQ ID NO: 58) |
| GALR1_sgRNA 1 | GGCGGTCGGGAACCTCAGCG (SEQ ID NO: 59) |
| GALR1_sgRNA 2 | GTTCCCGACCGCCAGCTCCA (SEQ ID NO: 60) |
| OR51D1_sgRNA 1 | TATGATAGGGACCAAGAGCT (SEQ ID NO: 61) |
| OR51D1_sgRNA 2 | ATGATAGGGACCAAGAGCTG (SEQ ID NO: 62) |
| MLH1_sgRNA 1 | ATTACAACGAAAACAGCTGA (SEQ ID NO: 63) |
| MLH1_sgRNA 2 | CTGATGGAAAGTGTGCATAC (SEQ ID NO: 64) |
| MSH2_sgRNA 1 | CGCGCTGCTGGCCGCCCGGG (SEQ ID NO: 65) |
| MSH2_sgRNA 2 | GGTCTTGAACACCTCCCGGG (SEQ ID NO: 66) |
| MSH2_sgRNA 3 | GTGAGGAGGTTTCGACATGG (SEQ ID NO: 67) |
| MSH6_sgRNA 1 | GAAGTACAGCCTAAGACACA (SEQ ID NO: 68) |
| MSH6_sgRNA 2 | AGCCTAAGACACAAGGATCT (SEQ ID NO: 69) |
| PMS2_sgRNA 1 | CGACTGATGTTTGATCACAA (SEQ ID NO: 70) |
| PMS2_sgRNA 2 | AGTTTCAACCTGAGTTAGGT (SEQ ID NO: 71) |
| CSPG4_sgRNA 1 | GAGTTAAGTGCGCGGACACC (SEQ ID NO: 72) |
| CSPG4_sgRNA 2 | CCACTCAGCTCCCAGCTCCC (SEQ ID NO: 73) |
| neg_sgRNA 1 | CAATAGCAAACCGGGGCAGT (SEQ ID NO: 74) |
| neg_sgRNA 2 | GTGACTCCATTACCAGGCTG (SEQ ID NO: 75) |

The effects of the sgRNA pools on cell viability against 6-TG treatment were quantified by a 3-(4,5-dimethyl-2-thiazolyl)-2,5-diphenyl-2-H-tetrazolium bromide (MTT) assay. Top 10 genes from CRISPR^{iBAR} as well as CRISPR screens were chosen for validation. Noticeably, two non-targeting control genes were identified and ranked in the top-ten candidate list from the conventional CRISPR screen. These evident false-positive results are predictable because of the high MOI we used to generate the cell library. We successfully confirmed that the top 10 candidate genes from CRISPR^{iBAR} of both replicates were all true-positive results; in contrast, only five genes from the top-ten candidate list from the conventional method turned out to be true positives (FIG. 11E). Among them, four genes (HPRT1, ITGB1, SRGAP2 and AKTIP) were obtained using both methods, whereas six genes (ACTR3C, PPP1R17, ACSBG1, CALM2, TCF21 and KIFAP3) were only identified and ranked at the top from CRISPR^{iBAR}. In summary, iBAR improved accuracy with lower false-positive and false-negative rates for high MOI screens compared with conventional method.

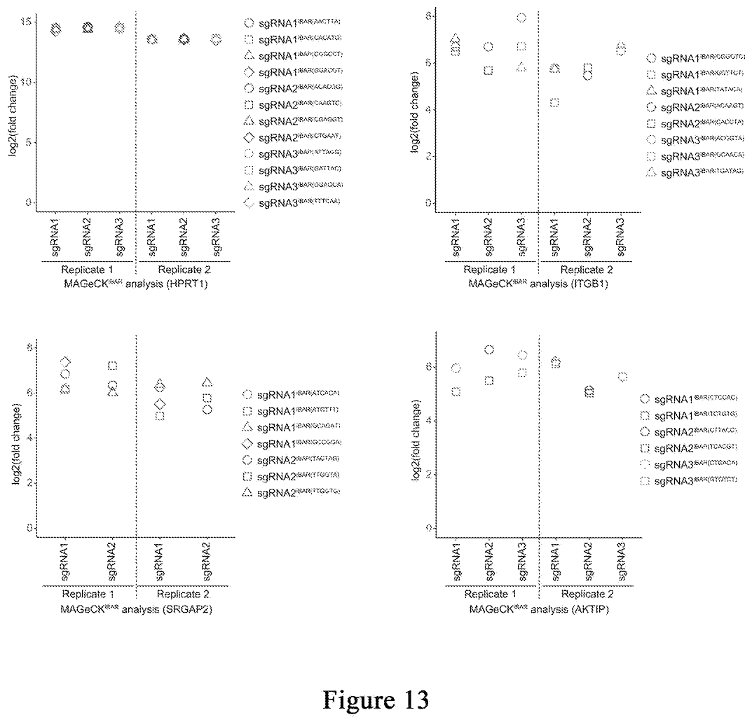

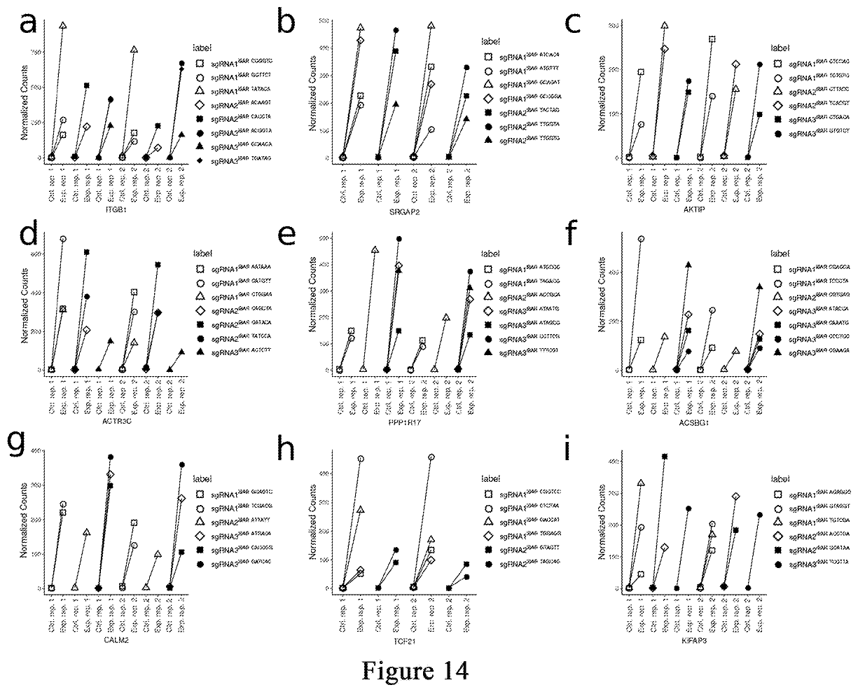

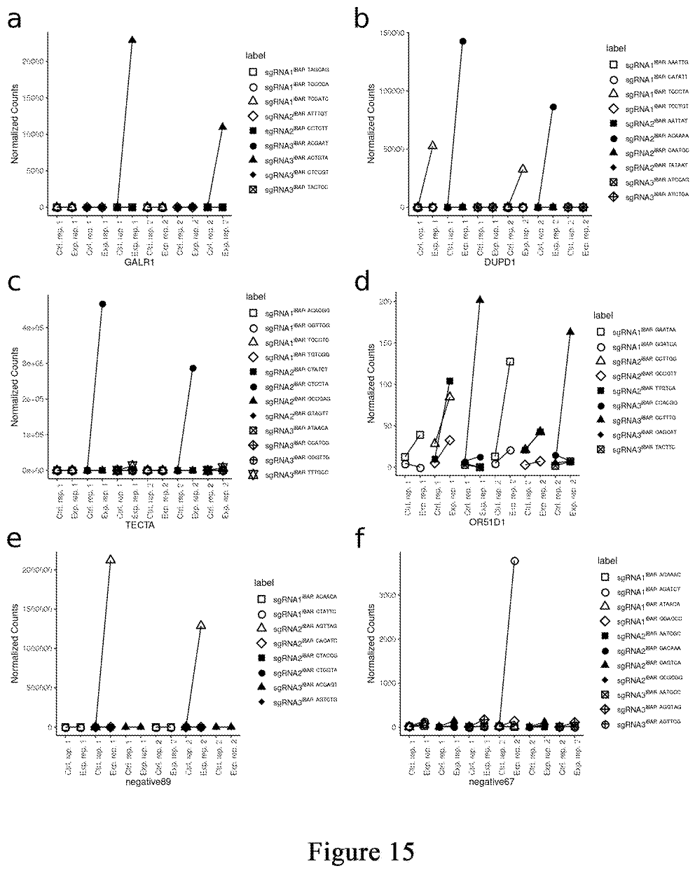

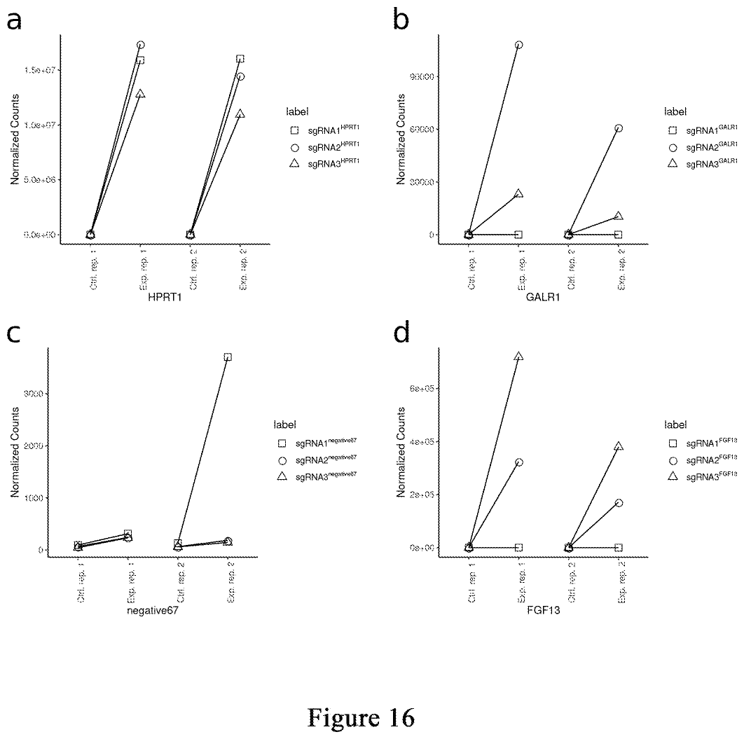

We further assessed the performance of each sgRNA^{iBAR} targeting the top four candidate genes (HPRT1, ITGB1, SRGAP2 and AKTIP). All the different iBARs of the enriched sgRNAs appeared to have little effect on the enrichment levels of their affiliated sgRNAs, and the order of iBARs associated with any particular sgRNA appeared to be random (FIG. 13), further supporting our prior notion that the iBARs did not affect the efficiency of their affiliated sgRNAs. All four HPRT1-targeting sgRNAs^{iBAR} were significantly enriched after 6-TG treatment in both replicates (FIG. 11F). Most sgRNAs^{iBAR} of other CRISPR^{iBAR} identified genes were enriched after 6-TG selection (FIG. 14). In contrast, only a very few of sgRNAs^{iBAR} of some top-ranked genes from conventional CRISPR screening were enriched, including FGF13 (FIG. 11G), GALR1 and two negative control genes (FIG. 15), leading to false-positive hits in the MAGeCK but not MAGeCK^{iBAR} analysis (FIG. 16).

Four barcodes for each sgRNA, as we designed, appeared to provide sufficient internal repeats to evaluate data consistency. The high level of consistency between the two biological replicates indicates that one experimental replicate is sufficient for CRISPR screens using the iBAR method (FIG. 6, FIG. 11D and Table 2). Because the library coverage was significantly increased with a high MOI in the transduction with a fixed number of cells for library construction, we decreased the starting cells for library construction more than 20-fold (MOI=3) and 70-fold (MOI=10) to match and even top the results from conventional screening at an MOI of 0.3 using two biological replicates (Table 4).

TABLE 4
Comparison of the number of cells required for CRISPR library
construction for TcdB screenings at different MOIs

| | | Cell number required |
|---|---|---|
| Screening methods with sgRNA | | for the construction |
| library constructed at different | Transduction | of the human whole- |
| MOIs | rate | genome library |
| CRISPR screening | 26% | 1.78 × 10⁸ (2 replicates)\| |
| (MOI ~ 0.3) | | 400× for each sgRNA |
| CRISPR^{iBAR} screening | 95% | 8.14 × 10⁶ (1 replicate)\| |
| (MOI ~ 3) | | 100× for each sgRNA^{iBAR} |
| CRISPR^{iBAR}screening | >99.9% | 2.32 × 10⁶ (1 replicate)\| |
| (MOI ~ 10) | | 100× for each sgRNA^{iBAR} |

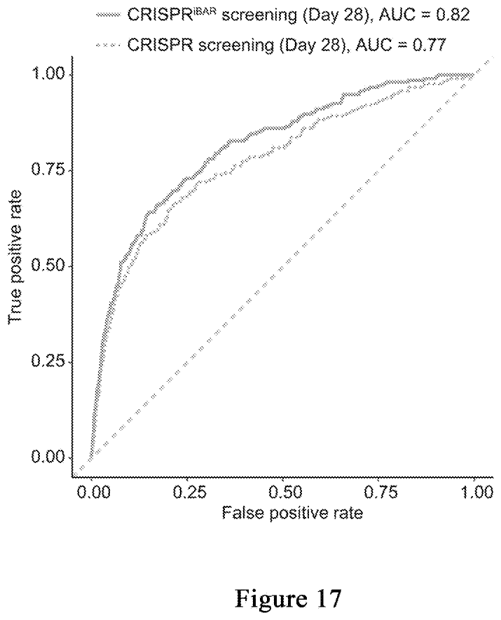

Because multiple cuttings decrease cell viability, CRISPR library constructed at a high MOI might have abnormal false discovery rate for negative screening^{23,24} We therefore performed a genome-scale negative screening at an MOI of 0.3 to assess iBAR method in calling essential genes. For positive screening using iBAR, we modified the model-estimated variance of sgRNAs with different fold change directions among barcodes to enlarge variance so that the mis-associated sgRNAs were subject to adequate penalty. For negative screening, however, sgRNA depletion through mis-association had little effect on its consistency of fold change directions as non-functional sgRNAs remained unchanged. Therefore, we treated barcodes only as internal replicates without the penalty procedure. We indeed achieved improved statistics with higher true positive and lower false positive rates for negative screening using iBAR method at a low MOI than the conventional approach using gold-standard essential genes²⁵ (FIG. 17).

In addition to the significant reduction in cells for library construction, the internal replicates offered by iBARs within the same experiment would lead to more uniform conditions and fairer comparisons versus separate biological replicates, consequently improving statistical scores. The advantage of the iBAR method would become greater when large-scale CRISPR screens in multiple cell lines are in demand or when the cell samples for screening are scarce (e.g., samples from patients or those of primary origin). Especially for in vivo screening in which the lentiviral transduction rate is hard to predict and variable conditions in different animals might greatly impact the screening outcomes, the iBAR method could be an ideal solution to resolve these technical limitations.

For negative screening, however, iBAR method improved statistics on library made of viral infection at a low MOI (FIG. 17). Notwithstanding the technical advancement of the iBAR method to offer the same benefit of internal replications, we must be cautious with the MOI during viral transduction to generate the original cell library in negative screens based on measuring cell viability. Although massive integrates have been reported not to affect cell fitness²⁶, multiple cuttings on DNA caused by higher MOI in cells with active Cas9 have been shown to reduce cell viability^{23,24}. Strategies without cuttings, such as CRISPRi/a⁹ or iSTOP systems²⁷, could be better choices to combine with the iBAR system for negative screening at a high MOI.

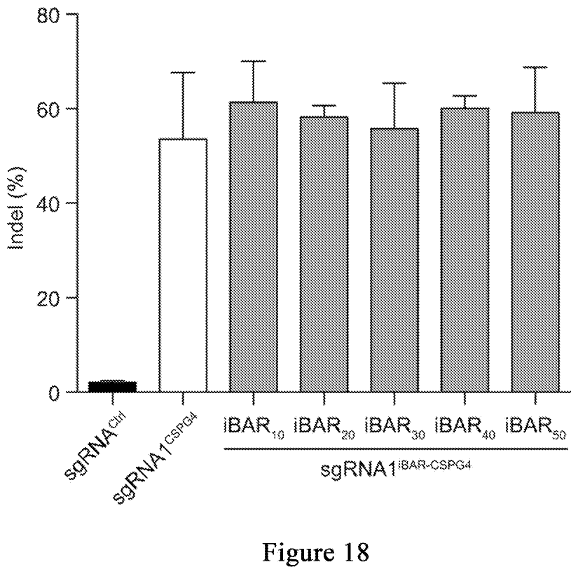

Although we had data to support that iBAR₆ had little effect on the activities of sgRNAs, we would not recommend to use barcodes with consecutive T (>4) so as to avoid any minor effects. Ultimately, 4,096 types of iBAR₆ provided sufficient varieties to make CRISPR libraries. In addition, the length of the iBAR is not limited to 6 nt. We have tested different lengths of iBARs, and found that their lengths could be up to 50-nt without affecting functions of their affiliated sgRNAs (FIG. 18). In addition, it is not necessary to design different barcode sets for different sgRNAs. A fixed set of iBARs assigned to all sgRNAs should work as well as random assignment in library screening. Our iBAR strategy with a streamlined analytic tool MAGeCK^{iBAR} would facilitate large-scale CRISPR screens for broad biomedical discoveries in various settings.

### REFERENCES

1. Jinek, M. et al. A programmable dual-RNA-guided DNA endonuclease in adaptive bacterial immunity. Science 337, 816-821 (2012).
2. Cong, L. et al. Multiplex genome engineering using CRISPR/Cas systems. Science 339, 819-823 (2013).
3. Mali, P. et al. RNA-guided human genome engineering via Cas9. Science 339, 823-826 (2013).
4. Shalem, O. et al. Genome-scale CRISPR-Cas9 knockout screening in human cells. Science 343, 84-87 (2014).
5. Wang, T., Wei, J. J., Sabatini, D. M. & Lander, E. S. Genetic screens in human cells using the CRISPR-Cas9 system. Science 343, 80-84 (2014).
6. Koike-Yusa, H., Li, Y., Tan, E. P., Velasco-Herrera Mdel, C. & Yusa, K. Genome-wide recessive genetic screening in mammalian cells with a lentiviral CRISPR-guide RNA library. Nat Biotechnol 32, 267-273 (2014).
7. Zhou, Y. et al. High-throughput screening of a CRISPR/Cas9 library for functional genomics in human cells. Nature 509, 487-491 (2014).
8. Zhu, S. et al. Genome-scale deletion screening of human long non-coding RNAs using a paired-guide RNA CRISPR-Cas9 library. Nat Biotechnol 34, 1279-1286 (2016).
9. Gilbert, L. A. et al. Genome-Scale CRISPR-Mediated Control of Gene Repression and Activation. Cell 159, 647-661 (2014).
10. Konermann, S. et al. Genome-scale transcriptional activation by an engineered CRISPR-Cas9 complex. Nature 517, 583-588 (2015).
11. Peng, J., Zhou, Y., Zhu, S. & Wei, W. High-throughput screens in mammalian cells using the CRISPR-Cas9 system. FEBS J 282, 2089-2096 (2015).
12. Zhu, S., Zhou, Y. & Wei, W. Genome-Wide CRISPR/Cas9 Screening for High-Throughput Functional Genomics in Human Cells. Methods Mol Biol 1656, 175-181 (2017).
13. Michlits, G. et al. CRISPR-UMI: single-cell lineage tracing of pooled CRISPR-Cas9 screens. Nat Methods 14, 1191-1197 (2017).
14. Schmierer, B. et al. CRISPR/Cas9 screening using unique molecular identifiers. Molecular systems biology 13, 945 (2017).
15. Shechner, D. M., Hacisuleyman, E., Younger, S. T. & Rinn, J. L. Multiplexable, locus-specific targeting of long RNAs with CRISPR-Display. Nat Methods 12, 664-670 (2015).
16. Bradley, K. A., Mogridge, J., Mourez, M., Collier, R. J. & Young, J. A. Identification of the cellular receptor for anthrax toxin. Nature 414, 225-229 (2001).
17. Li, W. et al. MAGeCK enables robust identification of essential genes from genome-scale CRISPR/Cas9 knockout screens. Genome Biol 15, 554 (2014).
18. Lyras, D. et al. Toxin B is essential for virulence of Clostridium difficile. Nature 458, 1176-1179 (2009).
19. Yuan, P. et al. Chondroitin sulfate proteoglycan 4 functions as the cellular receptor for Clostridium difficile toxin B. Cell Res 25, 157-168 (2015).
20. Tao, L. et al. Frizzled proteins are colonic epithelial receptors for C. difficile toxin B. Nature 538, 350-355 (2016).
21. Tan, Y. Y., Epstein, L. B. & Armstrong, R. D. In vitro evaluation of 6-thioguanine and alpha-interferon as a therapeutic combination in HL-60 and natural killer cells. Cancer Res 49, 4431-4434 (1989).
22. Duan, J., Nilsson, L. & Lambert, B. Structural and functional analysis of mutations at the human hypoxanthine phosphoribosyl transferase (HPRT1) locus. Human mutation 23, 599-611 (2004).
23. Jackson, S. P. Sensing and repairing DNA double-strand breaks. Carcinogenesis 23, 687-696 (2002).
24. Meyers, R. M. et al. Computational correction of copy number effect improves specificity of CRISPR-Cas9 essentiality screens in cancer cells. Nat Genet 49, 1779-1784 (2017).
25. Hart, T., Brown, K. R., Sircoulomb, F., Rottapel, R. & Moffat, J. Measuring error rates in genomic perturbation screens: gold standards for human functional genomics. Molecular systems biology 10, 733 (2014).
26. Zhou, Y. et al. Painting a specific chromosome with CRISPR/Cas9 for live-cell imaging. Cell Res 27, 298-301 (2017).
27. Billon, P. et al. CRISPR-Mediated Base Editing Enables Efficient Disruption of Eukaryotic Genes through Induction of STOP Codons. Mol Cell 67, 1068-1079 e1064 (2017).
28. Engler, C., Gruetzner, R., Kandzia, R. & Marillonnet, S. Golden gate shuffling: a one-pot DNA shuffling method based on type Its restriction enzymes. PLoS One 4, e5553 (2009).
29. Wei, W., Lu, Q., Chaudry, G. J., Leppla, S. H. & Cohen, S. N. The LDL receptor-related protein LRP6 mediates internalization and lethality of anthrax toxin. Cell 124, 1141-1154 (2006).
30. Qian, L. et al. Bidirectional effect of Wnt signaling antagonist DKK1 on the modulation of anthrax toxin uptake. Science China. Life sciences 57, 469-481 (2014).
31. Anders, S. & Huber, W. Differential expression analysis for sequence count data. Genome Biol 11, R106 (2010).
32. Robinson, M. D. & Smyth, G. K. Small-sample estimation of negative binomial dispersion, with applications to SAGE data. Biostatistics 9, 321-332 (2008).
33. Kolde, R., Laur, S., Adler, P. & Vilo, J. Robust rank aggregation for gene list integration and meta-analysis. Bioinformatics 28, 573-580 (2012).

What is claimed is:

1. A set of sgRNA^{iBAR} constructs comprising three or more sgRNA^{iBAR} constructs each comprising or encoding an sgRNA^{iBAR}, wherein each sgRNA^{iBAR} has an sgRNA^{iBAR} sequence comprising a guide sequence and an internal barcode (iBAR) sequence, wherein each guide sequence is complementary to a target genomic locus, wherein the guide sequences for the three or more sgRNA^{iBAR} constructs are the same, wherein the iBAR sequence for each of the three or more sgRNA^{iBAR} constructs is different from each other, and wherein each sgRNA^{iBAR} is operable with a Cas protein to modify the target genomic locus.

2. The set of sgRNA^{iBAR} constructs of claim 1, wherein each sgRNA^{iBAR} sequence comprises a first stem sequence and a second stem sequence, wherein the first stem sequence hybridizes with the second stem sequence to form a double-stranded RNA region that interacts with the Cas protein, and wherein the iBAR sequence is disposed between the first stem sequence and the second stem sequence.

3. The set of sgRNA^{iBAR} constructs of claim 1 or 2, wherein the Cas protein is Cas9.

4. The set of sgRNA^{iBAR} constructs of claim 3, wherein each sgRNA^{iBAR} sequence comprises a guide sequence fused to a second sequence, wherein the second sequence comprises a repeat-anti-repeat stem loop that interacts with the Cas9.

5. The set of sgRNA^{iBAR} constructs of claim 4, wherein the iBAR sequence of each sgRNA^{iBAR} sequence is disposed in the loop region of the repeat-anti-repeat stem loop.

6. The set of sgRNA^{iBAR} constructs of claim 4 or 5, wherein the second sequence of each sgRNA^{iBAR} sequence further comprises a stem loop 1, stem loop 2, and/or stem loop 3.

7. The set of sgRNA^{iBAR} constructs of any one of claims 1-6, wherein each iBAR sequence comprises about 1-50 nucleotides.

8. The set of sgRNA^{iBAR} constructs of any one of claims 1-7, wherein each guide sequence comprises about 17-23 nucleotides.

9. The set of sgRNA^{iBAR} constructs of any one of claims 1-8, wherein each sgRNA^{iBAR} construct is a plasmid.

10. The set of sgRNA^{iBAR} constructs of any one of claims 1-8, wherein each sgRNA^{iBAR} construct is a viral vector.

11. The set of sgRNA^{iBAR} constructs of claim 10, wherein the viral vector is a lentiviral vector.

12. The set of sgRNA^{iBAR} constructs of any one of claims 1-11, comprising four sgRNA^{iBAR} constructs, wherein the iBAR sequence for each of the four sgRNA^{iBAR} constructs is different from each other.

13. An sgRNA^{iBAR} library comprising a plurality of sets of sgRNA^{iBAR} constructs according to any one of claims 1-12, wherein each set corresponds to a guide sequence complementary to a different target genomic locus.

14. The sgRNA^{iBAR} library of claim 13, comprising at least about 1000 sets of sgRNA^{iBAR} constructs.

15. The sgRNA^{iBAR} library of claim 13 or 14, wherein the iBAR sequences for at least two sets of sgRNA^{iBAR} constructs are the same.

16. A method of preparing an sgRNA^{iBAR} library comprising a plurality of sets of sgRNA^{iBAR} constructs, wherein each set corresponds to one of a plurality of guide sequences complementary to different target genomic loci, wherein the method comprises:
a) designing three or more sgRNA^{iBAR} constructs for each guide sequence, wherein each sgRNA^{iBAR} construct comprises or encodes an sgRNA^{iBAR} having an sgRNA^{iBAR} sequence comprising the corresponding guide sequence and an iBAR sequence, wherein the iBAR sequence corresponding to each of the three or more sgRNA^{iBAR} constructs is different from each other, and wherein each sgRNA^{iBAR} is operable with a Cas protein to modify the corresponding target genomic locus; and
b) synthesizing each sgRNA^{iBAR} construct, thereby producing the sgRNA^{iBAR} library.

17. The method of claim 16, further comprising providing the plurality of guide sequences.

18. An sgRNA^{iBAR} library prepared using the method of claim 16 or 17.

19. A composition comprising the set of sgRNA^{iBAR} constructs according to any one of claims 1-12, or the sgRNA^{iBAR} library according to any one of claims 13-15 and 18.

20. A method of screening for a genomic locus that modulates a phenotype of a cell, comprising:
a) contacting an initial population of cells with i) the sgRNA^{iBAR} library of any one of claims 13-15 and 18; and optionally ii) a Cas component comprising a Cas protein or a nucleic acid encoding the Cas protein under a condition that allows introduction of the sgRNA^{iBAR} constructs and the optional Cas component into the cells to provide a modified population of cells;
b) selecting a population of cells having a modulated phenotype from the modified population of cells to provide a selected population of cells;
c) obtaining sgRNA^{iBAR} sequences from the selected population of cells;
d) ranking the corresponding guide sequences of the sgRNA^{iBAR} sequences based on sequence counts, wherein the ranking comprises adjusting the rank of each guide sequence based on data consistency among the iBAR sequences in the sgRNA^{iBAR} sequences corresponding to the guide sequence; and
e) identifying the genomic locus corresponding to a guide sequence ranked above a predetermined threshold level.

21. The method of claim 20, wherein the cell is a eukaryotic cell.

22. The method of claim 21, wherein the cell is a mammalian cell.

23. The method of any one of claims 20-22, wherein the initial population of cells expresses a Cas protein.

24. The method of any one of claims 20-23, wherein each sgRNA^{iBAR} construct is a viral vector, and wherein the sgRNA^{iBAR} library is contacted with the initial population of cells at a multiplicity of infection (MOI) of more than about 2.

25. The method of any one of claims 20-24, wherein more than about 95% of the sgRNA^{iBAR} constructs in the sgRNA^{iBAR} library are introduced into the initial population of cells.

26. The method of any one of claims 20-25, wherein the screening is carried out at more than about 1000-fold coverage.

27. The method of any one of claims 20-26, wherein the screening is positive screening.

28. The method of any one of claims 20-26, wherein the screening is negative screening.

29. The method of any one of claims 20-28, wherein the phenotype is protein expression, RNA expression, protein activity, or RNA activity.

30. The method of any one of claims 20-28, wherein the phenotype is selected from the group consisting of cell death, cell growth, cell motility, cell metabolism, drug resistance, drug sensitivity, and response to a stimulus.

31. The method of claim 30, wherein the phenotype is response to a stimulus, and wherein the stimulus is selected from the group consisting of a hormone, a growth factor, an inflammatory cytokine, an anti-inflammatory cytokine, a drug, a toxin, and a transcription factor.

32. The method of any one of claims 20-31, wherein the sgRNA^{iBAR} sequences are obtained by genome sequencing or RNA sequencing.

33. The method of claim 32, wherein the sgRNA^{iBAR} sequences are obtained by next-generation sequencing.

34. The method of any one of claims 20-33, wherein the sequence counts are subject to median ratio normalization followed by mean-variance modeling.

35. The method of claim 34, wherein the variance of each guide sequence is adjusted based on data consistency among the iBAR sequences in the sgRNA^{iBAR} sequences corresponding to the guide sequence.

36. The method of any one of claims 20-35, wherein the sequence counts obtained from the selected population of cells are compared to corresponding sequence counts obtained from a population of control cells to provide fold changes.

37. The method of claim 36, wherein the data consistency among the iBAR sequences in the sgRNA^{iBAR} sequences corresponding to each guide sequence is determined based on the direction of the fold change of each iBAR sequence, wherein the variance of the guide sequence is increased if the fold changes of the iBAR sequences are in opposite directions with respect to each other.

38. The method of any one of claims 20-37, further comprising validating the identified genomic locus.

39. A kit for screening a genomic locus that modulates a phenotype of a cell, comprising the sgRNA1^{AR} library of any one of claims 13-15 and 18.

40. The kit of claim 39, further comprises a Cas protein or a nucleic acid encoding the Cas protein.
